(19) United States
(12) Patent Application Publication
(10) Pub. No.: US 2021/0284647 A1
(43) Pub. Date: Sep. 16, 2021

(54) N-(3-(IMIDAZO[1,2-B]PYRIDAZIN-3-YLETHYNYL)-4-METHYLPHENYL)-5-PHENYL-4,5-DIHYDRO-1H-PYRAZOLE-1-CARBOXAMIDE DERIVATIVE, AND PHARMACEUTICAL COMPOSITION CONTAINING SAME AS ACTIVE INGREDIENT FOR TREATING KINASE-RELATED DISEASES

(71) Applicants: VORONOIBIO INC., Incheon (KR); VORONOI CO., LTD, Incheon (KR)

(72) Inventors: Hua LI, Incheon (KR); Seohyun JO, Incheon (KR); Woomi DO, Incheon (KR); Hyunkyung KIM, Incheon (KR); Jieun CHOI, Incheon (KR); Jung Beom SON, Incheon (KR); Namdoo KIM, Incheon (KR)

(21) Appl. No.: 17/258,667

(22) PCT Filed: Jul. 5, 2019
(86) PCT No.: PCT/KR2019/008288

§ 371 Date: Jan. 7, 2021

(30) Foreign Application Priority Data
Jul. 10, 2018 (KR) 10-2018-0080065
May 8, 2019 (KR) 10-2019-0053957

(51) Int. Cl. C07D 487/04; C07D 471/04

(52) U.S. Cl. CPC C07D 487/04; C07D 471/04

(57) ABSTRACT

The N-(3-(imidazo[1,2-b]pyridazin-3-ylethynyl)-4-methylphenyl)-5-phenyl-4,5-dihydro-1H-pyrazole-1-carboxamide derivative exhibits excellent inhibitory activity against at least one kinase selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK, and thus being useable as a therapeutic agent for kinase-related disease.

This patent application is the National Stage of International Application No. PCT/KR2019/008288 filed Jul. 5, 2019, which claims the benefit of priority from Korean Patent Application No. 10-2018-0080065 (filed on Jul. 10, 2018) and No. 10-2019-0053957 (filed on May 8, 2019), the content of each of which is incorporated herein by reference.

### BACKGROUND OF THE INVENTION

### 1. Field of the Invention

The present invention relates to a N-(3-(imidazo[1,2-b]pyridazin-3-ylethynyl)-4-methylphenyl)-5-phenyl-4,5-dihydro-1H-pyrazole-1-carboxamide derivative and a pharmaceutical composition comprising the same as an active ingredient for treating kinase-related disease.

### 2. Description of the Related Art

Protein kinase is an enzyme that catalyzes the phosphorylation reaction to transfer the gamma-phosphate group of ATP to the hydroxy groups of tyrosine, serine and threonine, and it is responsible for cell metabolism, gene expression, cell growth, differentiation and cell division. It also plays an important role in cellular signaling (Non-patent reference 1, Thomas A. Hamilton, in Encyclopedia of Immunology (Second Edition), 1998, 2028-2033). Protein kinases are classified into tyrosine protein kinases and serine/threonine kinases. More than about 90 protein kinases are tyrosine kinases.

Protein kinases must be smoothly regulated in the transition between the active and inactive states by a molecular switch in cells. If the transition between the active and inactive states is abnormally regulated, intracellular signal transduction is excessively activated, leading to uncontrolled cell division and proliferation. In addition, abnormal activation of protein kinase caused by gene mutation, amplification, and overexpression is related to the development and progression of various tumors, and thus plays a crucial role in the development of various diseases such as inflammatory diseases, degenerative brain diseases, autoimmune diseases and cancer. Examples of kinases related this include ABL1, ABL2, BRAF, CDK11, CDK8, CDKL2, CIT, CSF1R, DDR1, DDR2, FLT3, KIT, LOK, LTK, MUSK, PAK3, PDGFRA, PDGFRB, RAF1, RIPK1, and the like.

RIPK1 (receptor-interacting protein kinase 1), a specific example of protein kinase, is a multifunctional signal transducer involved in mediating NF-κB activation, apoptosis, and/or necroptosis.

In particular, RIP1 kinase activity is known to be critically involved in mediating necroptosis, a caspase-independent pathway of necrotic cell death (Non-patent reference 2, Holler et al., Nat Immunol 2000; 1: 489-495; Non-patent reference 3, Degterev et al., Nat Chem Biot 2008; 4: 313-321).

Therefore, if the RIP1 kinase activity can be effectively inhibited, cell protection is possible under the condition of inducing apoptosis by tumor necrosis factor-alpha (TNF-α), thereby blocking necroptosis.

Necroptosis mediated by the RIP1 kinase has been reported to be related with various diseases such as inflammatory diseases, degenerative brain diseases, autoimmune diseases, and cancer.

First, RIP3 knockout mice designed to completely block RIP1K-mediated necrosis are found to be protective against inflammatory bowel disease (including ulcerative colitis and Crohn's disease) (Non-patent reference 4, (2011) Nature 477, 330-334), psoriasis (Non-patent reference 5, (2011) Immunity 35, 572-582), retinal-detachment-induced photoreceptor necrosis (Non-patent reference 6, (2010) PNAS 107, 21695-21700), retinitis pigmentosa (Non-patent reference 7, (2012) Proc. Natl. Acad. Sci., 109:36, 14598-14603), cerulein-induced acute pancreatitis (Non-patent reference 8, (2009) Cell 137, 1100-1111) and sepsis/systemic inflammatory response syndrome (SIRS) (Non-patent reference 9, (2011) Immunity 35, 908-918). Therefore, if a specific compound can block necroptosis mediated by RIP1 kinase, the compound has the potential to be developed as a therapeutic agent for inflammatory diseases.

In addition, RIP1 kinase has been known to mediate microglial responses in Alzheimer's disease (Non-patent reference 10, PNAS Oct. 10, 2017. 114 (41) E8788-E8797). Therefore, if a specific compound can effectively inhibit the activity of RIP1 kinase by targeting thereof, the compound can be developed as a therapeutic agent for degenerative brain diseases (i.e., neurodegenerative diseases) such as Alzheimer's disease, Down's syndrome, Parkinson's disease, Lou Gehrig's disease, dementia, Huntington's disease, multiple sclerosis, proximal lateral sclerosis, stroke, and mild cognitive impairment.

Furthermore, RIP1 kinase is known to regulate the production of tumor necrosis factor-alpha (TNF-α), and TNF-α is known as a pro-inflammatory cytokine involved in cell death and inflammation mediation in numerous diseases such as rheumatoid arthritis and cancer (Non-patent reference 11, Cell Death and Disease (2012) 3, e320). Therefore, if a specific compound can effectively inhibit the activity of RIP1 kinase by targeting thereof, the compound can be developed as a therapeutic agent for autoimmune diseases such as rheumatoid polymyalgia including rheumatoid arthritis, ankylosing spondylitis, motor neuron disease, or cancer.

In addition, RIP1 kinase has been known to induce macrophage-mediated adaptive immune tolerance in pancreatic cancer (Non-patent reference 12, Cancer Cell 34, 757-774, Nov. 12, 2018). More specifically, it is known that RIP1K inhibition in TAMs leads to cytotoxic T cell activation against the mixed Th1/Th17 phenotype and T helper cell differentiation, leading to tumor immunity in organ type models of mouse and human PDA.

Accordingly, since targeting RIP1K synergistically with PD-1 and inducible co-stimulator-based immunotherapy, it can be seen that RIP1K is a checkpoint kinase that governs tumor immunity.

In other words, the compound that can effectively inhibit the activity of protein kinase including RIP1K is likely to be developed as a therapeutic agent for various diseases such as inflammatory diseases, degenerative brain diseases, autoimmune diseases, and cancer, and thus development of a protein kinase inhibitor having a novel structure is required.

### SUMMARY OF THE INVENTION

It is an object of the present invention to provide a N-(3-(imidazo[1,2-b]pyridazin-3-ylethynyl)-4-methylphenyl)-5-phenyl-4,5-dihydro-1H-pyrazole-1-carboxamide derivative having a novel structure, a stereoisomer thereof, a hydrate thereof, or a pharmaceutically acceptable salt thereof, which exhibits excellent inhibitory activity against various kinases and has a therapeutic effect on kinase-related disease.

It is another object of the present invention to provide a pharmaceutical composition comprising the N-(3-(imidazo[1,2-b]pyridazin-3-ylethynyl)-4-methylphenyl)-5-phenyl-4,5-dihydro-1H-pyrazole-1-carboxamide derivative, the stereoisomer thereof, the hydrate thereof, or the pharmaceutically acceptable salt thereof as an active ingredient for the prevention or treatment of kinase-related disease.

It is another object of the present invention to provide a health functional food composition comprising the N-(3-(imidazo[1,2-b]pyridazin-3-ylethynyl)-4-methylphenyl)-5-phenyl-4,5-dihydro-1H-pyrazole-1-carboxamide derivative, the stereoisomer thereof, the hydrate thereof, or the pharmaceutically acceptable salt thereof as an active ingredient for the prevention or amelioration of kinase-related disease.

It is another object of the present invention to provide a method for treating kinase-related disease comprising a step of administering the N-(3-(imidazo[1,2-b]pyridazin-3-ylethynyl)-4-methylphenyl)-5-phenyl-4,5-dihydro-1H-pyrazole-1-carboxamide derivative, the stereoisomer thereof, the hydrate thereof, or the pharmaceutically acceptable salt thereof to a subject in need thereof.

It is another object of the present invention to provide the N-(3-(imidazo[1,2-b]pyridazin-3-ylethynyl)-4-methylphenyl)-5-phenyl-4,5-dihydro-1H-pyrazole-1-carboxamide derivative, the stereoisomer thereof, the hydrate thereof, or the pharmaceutically acceptable salt thereof for use in the prevention or treatment of kinase-related disease.

It is another object of the present invention to provide a use of the N-(3-(imidazo[1,2-b]pyridazin-3-ylethynyl)-4-methylphenyl)-5-phenyl-4,5-dihydro-1H-pyrazole-1-carboxamide derivative, the stereoisomer thereof, the hydrate thereof, or the pharmaceutically acceptable salt thereof for preparing a medicament for use in the prevention or treatment of kinase-related disease.

To achieve the above objects, in one aspect of the present invention, the present invention provides a compound represented by formula 1, a stereoisomer thereof, a hydrate thereof or a pharmaceutically acceptable salt thereof.

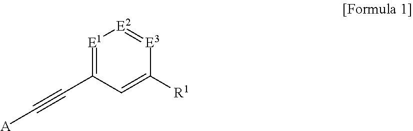

(In formula 1,

E¹ is ═CA¹- or ═N—,

A¹ is —H, C₁₋₁₀ straight or branched alkyl or halogen;

E² is ═CA²- or ═N—,

A² is —H, C₁₋₁₀ straight or branched alkyl or halogen;

E³ is ═CA³- or ═N—,

A³ is —H, halogen or

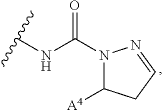

wherein, A⁴ is nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl or the substituted 5-10 membered heteroaryl is C₆₋₁₀ aryl or 5-10 membered heteroaryl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen;

R¹ is —H,

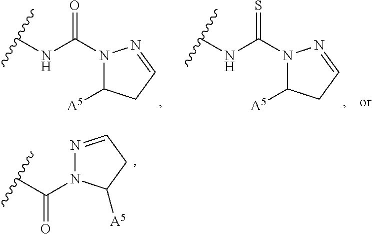

wherein, A⁵ is nonsubstituted, substituted or fused C₆₋₁₀ aryl, nonsubstituted, substituted or fused 5-10 membered heteroaryl containing one or more heteroatoms selected from the group consisting of N, O and S, or nonsubstituted or substituted C₄₋₁₀ cycloalkyl,

wherein, the substituted C₆₋₁₀ aryl, the substituted 5-10 membered heteroaryl, or the substituted C₄₋₁₀ cycloalkyl is C₆₋₁₀ aryl, 5-10 membered heteroaryl, or C₄₋₁₀ cycloalkyl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen,

wherein, the fused C₆₋₁₀ aryl or the fused 5-10 membered heteroaryl is C₆₋₁₀ aryl or 5-10 membered heteroaryl fused with 5-6 membered heterocycloalkyl containing one or more Os or phenyl; and

A is

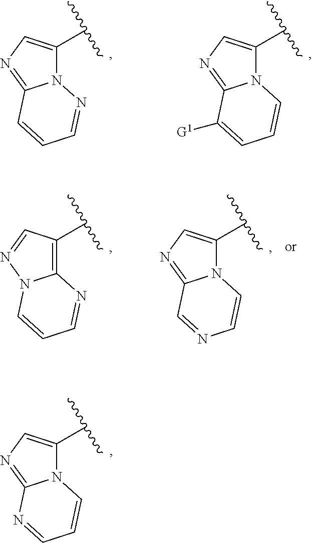

wherein, G¹ is hydrogen, halogen, hydroxy, nitro, C₁₋₁₀ straight or branched alkyl, C₁₋₁₀ straight or branched alkoxy or —NR⁴R⁵,

wherein, R⁴ and R⁵ are independently hydrogen, C₁₋₁₀ straight or branched alkylcarbonyl, C₁₋₁₀ straight or branched alkylaminocarbonyl, C₃₋₆ cycloalkylcarbonyl, C₁₋₁₀ straight or branched alkoxycarbonyl, nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl is C₆₋₁₀ aryl substituted with C₁₋₁₀ straight or branched alkylsulfonyl,

wherein, the substituted 5-10 membered heteroaryl is 5-10 membered heteroaryl substituted with C₁₋₁₀ straight or branched alkyl).

In another aspect of the present invention, the present invention provides a pharmaceutical composition comprising the N-(3-(imidazo[1,2-b]pyridazin-3-ylethynyl)-4-methylphenyl)-5-phenyl-4,5-dihydro-1H-pyrazole-1-carboxamide derivative represented by formula 1, the stereoisomer thereof, the hydrate thereof, or the pharmaceutically acceptable salt thereof as an active ingredient for the prevention or treatment of kinase-related disease.

In another aspect of the present invention, the present invention provides a health functional food composition comprising the N-(3-(imidazo[1,2-b]pyridazin-3-ylethynyl)-4-methylphenyl)-5-phenyl-4,5-dihydro-1H-pyrazole-1-carboxamide derivative represented by formula 1, the stereoisomer thereof, the hydrate thereof, or the pharmaceutically acceptable salt thereof as an active ingredient for the prevention or amelioration of kinase-related disease.

In another aspect of the present invention, the present invention provides a method for treating kinase-related disease comprising a step of administering the N-(3-(imidazo[1,2-b]pyridazin-3-ylethynyl)-4-methylphenyl)-5-phenyl-4,5-dihydro-1H-pyrazole-1-carboxamide derivative represented by formula 1, the stereoisomer thereof, the hydrate thereof, or the pharmaceutically acceptable salt thereof to a subject in need thereof.

In another aspect of the present invention, the present invention provides the N-(3-(imidazo[1,2-b]pyridazin-3-ylethynyl)-4-methylphenyl)-5-phenyl-4,5-dihydro-1H-pyrazole-1-carboxamide derivative represented by formula 1, the stereoisomer thereof, the hydrate thereof, or the pharmaceutically acceptable salt thereof for use in the prevention or treatment of kinase-related disease.

In another aspect of the present invention, the present invention provides a use of the N-(3-(imidazo[1,2-b]pyridazin-3-ylethynyl)-4-methylphenyl)-5-phenyl-4,5-dihydro-1H-pyrazole-1-carboxamide derivative represented by formula 1, the stereoisomer thereof, the hydrate thereof, or the pharmaceutically acceptable salt thereof for preparing a medicament for use in the prevention or treatment of kinase-related disease.

### Advantageous Effect

The N-(3-(imidazo[1,2-b]pyridazin-3-ylethynyl)-4-methylphenyl)-5-phenyl-4,5-dihydro-1H-pyrazole-1-carboxamide derivative provided in one aspect of the present invention has the effect of exhibiting excellent inhibitory activity against at least one kinase selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK, and thus being useable as a therapeutic agent for kinase-related disease.

### DESCRIPTION OF THE PREFERRED EMBODIMENTS

Hereinafter, the present invention is described in detail.

In one aspect of the present invention, the present invention provides a compound represented by formula 1, a stereoisomer thereof, a hydrate thereof or a pharmaceutically acceptable salt thereof.

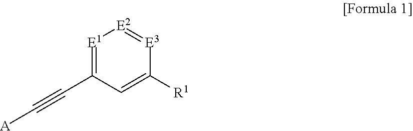

(In formula 1,

E¹ is ═CA¹- or ═N—,

A¹ is —H, C₁₋₁₀ straight or branched alkyl or halogen;

E² is ═CA²- or ═N—,

A² is —H, C₁₋₁₀ straight or branched alkyl or halogen;

E³ is ═CA³- or ═N—,

A³ is —H, halogen or

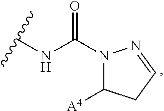

wherein, A⁴ is nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl or the substituted 5-10 membered heteroaryl is C₆₋₁₀ aryl or 5-10 membered heteroaryl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen;

R¹ is —H,

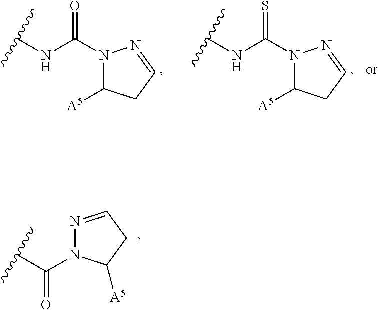

wherein, A⁵ is nonsubstituted, substituted or fused C₆₋₁₀ aryl, nonsubstituted, substituted or fused 5-10 membered heteroaryl containing one or more heteroatoms selected from the group consisting of N, O and S, or nonsubstituted or substituted C₄₋₁₀ cycloalkyl,

wherein, the substituted C₆₋₁₀ aryl, the substituted 5-10 membered heteroaryl, or the substituted C₄₋₁₀ cycloalkyl is C₆₋₁₀ aryl, 5-10 membered heteroaryl, or C₄₋₁₀ cycloalkyl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen,

wherein, the fused C₆₋₁₀ aryl or the fused 5-10 membered heteroaryl is C₆₋₁₀ aryl or 5-10 membered heteroaryl fused with 5-6 membered heterocycloalkyl containing one or more Os or phenyl; and

A is

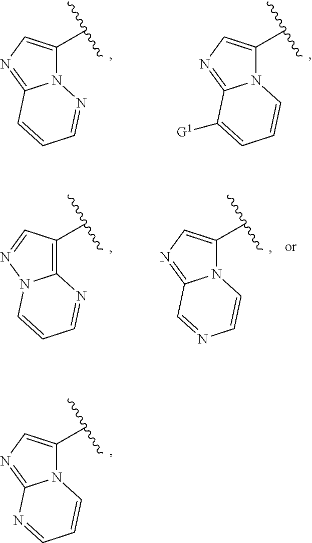

wherein, G¹ is hydrogen, halogen, hydroxy, nitro, C₁₋₁₀ straight or branched alkyl, C₁₋₁₀ straight or branched alkoxy or —NR⁴R⁵,

wherein, R⁴ and R⁵ are independently hydrogen, C₁₋₁₀ straight or branched alkylcarbonyl, C₁₋₁₀ straight or branched alkylaminocarbonyl, C₃₋₆ cycloalkylcarbonyl, C₁₋₁₀ straight or branched alkoxycarbonyl, nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl is C₆₋₁₀ aryl substituted with C₁₋₁₀ straight or branched alkylsulfonyl,

wherein, the substituted 5-10 membered heteroaryl is 5-10 membered heteroaryl substituted with C₁₋₁₀ straight or branched alkyl).

In another aspect of the present invention,

E¹ is ═CA¹- or ═N—,

A¹ is —H, C₁₋₅ straight or branched alkyl, or halogen;

E² is ═CA²- or ═N—,

A² is —H, C₁₋₅ straight or branched alkyl, or halogen;

E³ is ═CA³- or ═N—,

A³ is —H, halogen, or

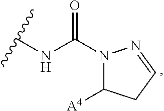

wherein, A⁴ is nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl or the substituted 5-10 membered heteroaryl is C₆₋₁₀ aryl or 5-10 membered heteroaryl substituted with one or more substituents selected from the group consisting of C₁₋₅ straight or branched alkyl nonsubstituted or substituted with one or more halogens, C₁₋₅ straight or branched alkoxy nonsubstituted or substituted with one or more halogens, and halogen;

R¹ is —H,

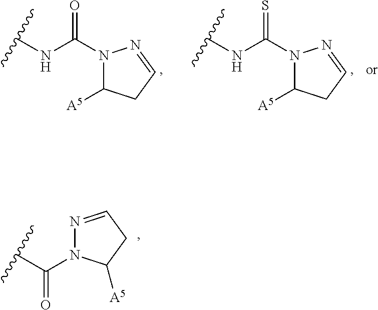

wherein, A⁵ is nonsubstituted, substituted or fused C₆₋₁₀ aryl, nonsubstituted, substituted or fused 5-10 membered heteroaryl containing one or more heteroatoms selected from the group consisting of N, O and S, or nonsubstituted or substituted C₄₋₁₀ cycloalkyl,

wherein, the substituted C₆₋₁₀ aryl, the substituted 5-10 membered heteroaryl, or the substituted C₄₋₁₀ cycloalkyl is C₆₋₁₀ aryl, 5-10 membered heteroaryl, or C₄₋₁₀ cycloalkyl substituted with one or more substituents selected from the group consisting of C₁₋₅ straight or branched alkyl nonsubstituted or substituted with one or more halogens, C₁₋₅ straight or branched alkoxy nonsubstituted or substituted with one or more halogens, and halogen,

wherein, the fused C₆₋₁₀ aryl or the fused 5-10 membered heteroaryl is C₆₋₁₀ aryl or 5-10 membered heteroaryl fused with 5 membered heterocycloalkyl containing two Os or phenyl; and

A is

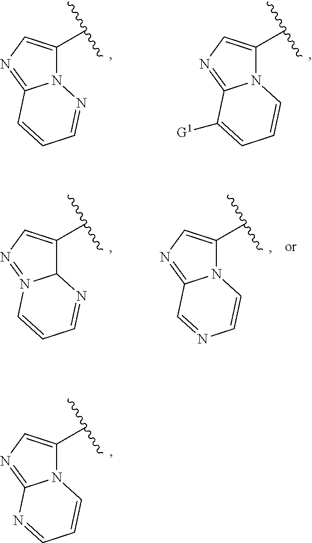

wherein, G¹ is hydrogen, halogen, hydroxy, nitro, C₁₋₅ straight or branched alkyl, C₁₋₅ straight or branched alkoxy or —NR⁴R⁵,

wherein, R⁴ and R⁵ are independently hydrogen, C₁₋₅ straight or branched alkylcarbonyl, C₁₋₅ straight or branched alkylaminocarbonyl, C₃₋₆ cycloalkylcarbonyl, C₁₋₅ straight or branched alkoxycarbonyl, nonsubstituted or substituted C₆₋₁₀ aryl, or nonsubstituted or substituted 5-10 membered heteroaryl containing one or more heteroatoms selected from the group consisting of N, O and S,

wherein, the substituted C₆₋₁₀ aryl is C₆₋₁₀ aryl substituted with C₁₋₅ straight or branched alkylsulfonyl,

wherein, the substituted 5-10 membered heteroaryl is 5-10 membered heteroaryl substituted with C₁₋₅ straight or branched alkyl.

In another aspect of the present invention,

E¹ is ═CA¹- or ═N—, wherein A¹ is —H, —CH₃, or —F;

E² is ═CA²- or ═N—, wherein A² is —H;

E³ is ═CA³- or ═N—, wherein A³ is —H, —F, or

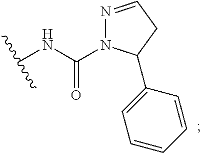 [SMILES: *NC(=O)N1N=CCC1c1ccccc1]

R¹ is —H,

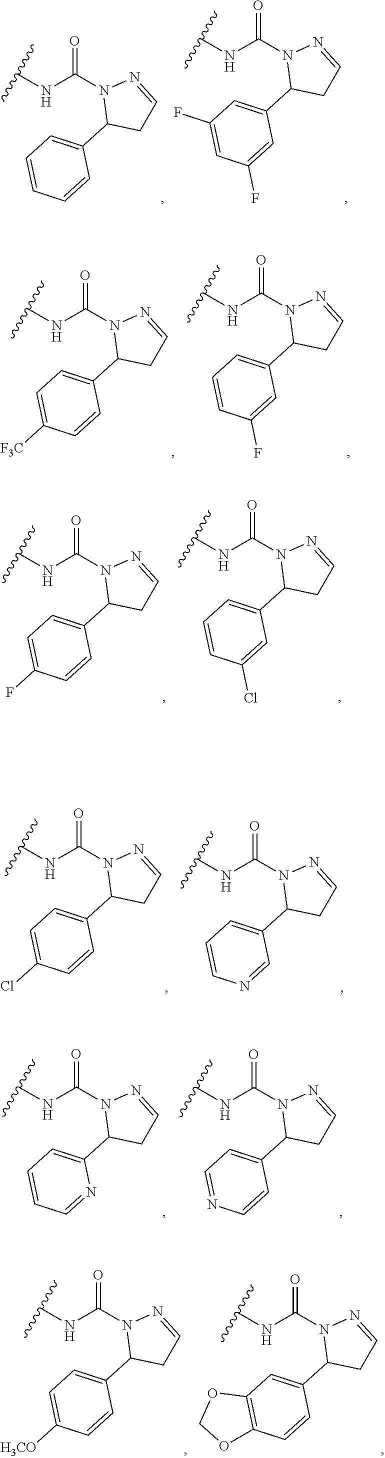

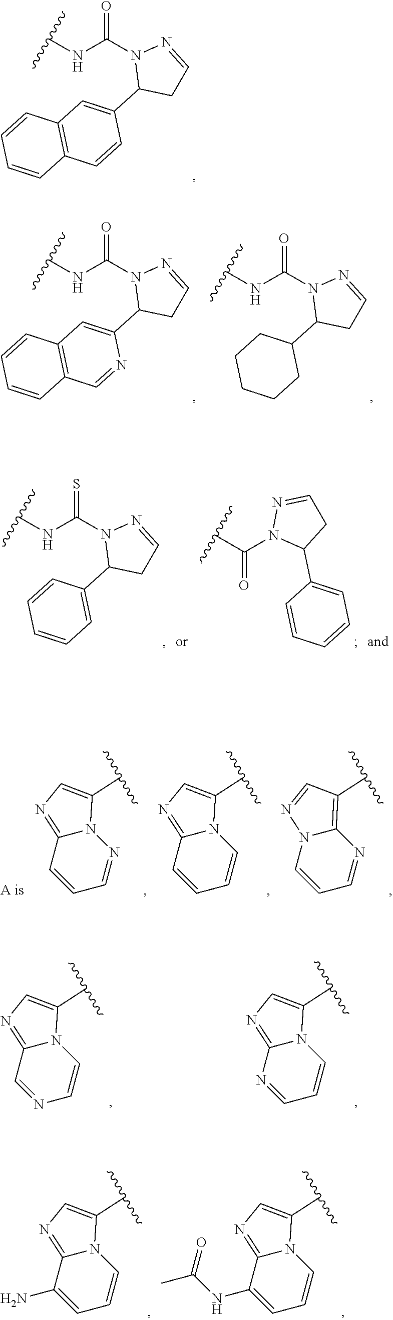

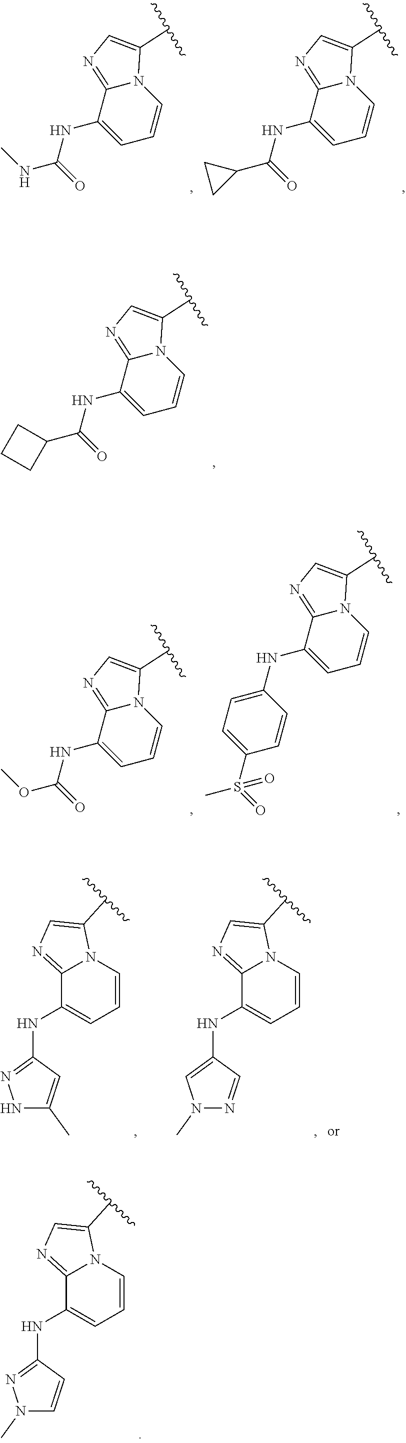

In one aspect of the present invention, the compound represented by formula 1, the stereoisomer thereof, the hydrate thereof, or the pharmaceutically acceptable salt thereof can be any compound selected from the group consisting of the following compounds.

(1) N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;

(2) (S)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;

(3) (R)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;

(4) 5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(5) (S)-5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(6) (R)-5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(7) N-(3-(imidazo[1,2-b]pyridazin-3-ylethynyl)-4-methylphenyl)-5-(4-(trifluoromethyl)phenyl)-4,5-dihydro-1H-pyrazole-1-carboxamide 2,2,2-trifluoroacetate;

(8) 5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(9) (S)-5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(10) (R)-5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(11) 5-(4-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(12) (S)-5-(4-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(13) (R)-5-(4-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(14) 5-(3-chlorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(15) 5-(4-chlorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;

(16) N-(3-(imidazo[1,2-b]pyridazin-3-ylethynyl)-4-methylphenyl)-5-(pyridin-3-yl)-4,5-dihydro-1H-pyrazole-1-carboxamide 2,2,2-trifluoroacetate;

(17) N-(3-(imidazo[1,2-b]pyridazin-3-ylethynyl)-4-methylphenyl)-5-(pyridin-2-yl)-4,5-dihydro-1H-pyrazole-1-carboxamide 2,2,2-trifluoroacetate;

(18) N-(3-(imidazo[1,2-b]pyridazin-3-ylethynyl)-4-methylphenyl)-5-(pyridin-4-yl)-4,5-dihydro-1H-pyrazole-1-carboxamide 2,2,2-trifluoroacetate;

(19) N-(3-(imidazo[1,2-b]pyridazin-3-ylethynyl)phenyl)-5-phenyl-4,5-dihydro-1H-pyrazole-1-carboxamide 2,2,2-trifluoroacetate;

(20) (S)—N-(3-(imidazo[1,2-b]pyridazin-3-ylethynyl)phenyl)-5-phenyl-4,5-dihydro-1H-pyrazole-1-carboxamide hydrochloride;

(21) (R)—N-(3-(imidazo[1,2-b]pyridazin-3-ylethynyl)phenyl)-5-phenyl-4,5-dihydro-1H-pyrazole-1-carboxamide hydrochloride;

(22) N-(4-(imidazo[1,2-b]pyridazin-3-ylethynyl)phenyl)-5-phenyl-4,5-dihydro-1H-pyrazole-1-carboxamide 2,2,2-trifluoroacetate;

(23) 5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;

(24) (S)-5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;

(25) (R)-5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;

(26) N-(3-(imidazo[1,2-b]pyridazin-3-ylethynyl)phenyl)-5-(4-(trifluoromethyl)phenyl)-4,5-dihydro-1H-pyrazole-1-carboxamide 2,2,2-trifluoroacetate;

(27) N-(3-(imidazo[1,2-b]pyridazin-3-ylethynyl)phenyl)-5-(4-methoxyphenyl)-4,5-dihydro-1H-pyrazole-1-carboxamide 2,2,2-trifluoroacetate;

(28) 5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(29) (S)-5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(30) (R)-5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(31) 5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(32) (S)-5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(33) (R)-5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;

(34) 5-(3-chlorophenyl)-N-(3-(imidazo[1,2-b]pyridazin-3-ylethynyl)phenyl)-4,5-dihydro-1H-pyrazole-1-carboxamide 2,2,2-trifluoroacetate;

(35) 5-(4-chlorophenyl)-N-(3-(imidazo[1,2-b]pyridazin-3-ylethynyl)phenyl)-4,5-dihydro-1H-pyrazole-1-carboxamide 2,2,2-trifluoroacetate;

(36) N-(3-(imidazo[1,2-b]pyridazin-3-ylethynyl)phenyl)-5-(pyridin-3-yl)-4,5-dihydro-1H-pyrazole-1-carboxamide 2,2,2-trifluoroacetate;

(37) N-(3-(imidazo[1,2-b]pyridazin-3-ylethynyl)phenyl)-5-(pyridin-2-yl)-4,5-dihydro-1H-pyrazole-1-carboxamide 2,2,2-trifluoroacetate;

(38) N-(3-(imidazo[1,2-b]pyridazin-3-ylethynyl)phenyl)-5-(pyridin-4-yl)-4,5-dihydro-1H-pyrazole-1-carboxamide hydrochloride;

(39) 5-(benzo[d][1,3]dioxol-5-yl)-N-(3-(imidazo[1,2-b]pyridazin-3-ylethynyl)phenyl)-4,5-dihydro-1H-pyrazole-1-carboxamide 2,2,2-trifluoroacetate; (40) N-(3-(imidazo[1,2-b]pyridazin-3-ylethynyl)phenyl)-5-(naphthalen-2-yl)-4,5-dihydro-1H-pyrazole-1-carboxamide;

(41) N-(3-(imidazo[1,2-b]pyridazin-3-ylethynyl)phenyl)-5-(isoquinolin-3-yl)-4,5-dihydro-1H-pyrazole-1-carboxamide;

(42) 5-cyclohexyl-N-(3-(imidazo[1,2-b]pyridazin-3-ylethynyl)phenyl)-4,5-dihydro-1H-pyrazole-1-carboxamide;

(43) N-(3-(imidazo[1,2-b]pyridazin-3-ylethynyl)phenyl)-5-phenyl-4,5-dihydro-1H-pyrazole-1-carbothioamide;

(44) N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;

(45) (S)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;

(46) (R)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;

(47) N-(2-fluoro-5-(imidazo[1,2-b]pyridazin-3-ylethynyl)phenyl)-5-phenyl-4,5-dihydro-1H-pyrazole-1-carboxamide 2,2,2-trifluoroacetate;

(48) N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;

(49) (S)—N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;

(50) (R)—N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;

(51) N-(5-(imidazo[1,2-b]pyridazin-3-ylethynyl)pyridin-3-yl)-5-phenyl-4,5-dihydro-1H-pyrazole-1-carboxamide 2,2,2--trifluoroacetate;

(52) N-(4-(imidazo[1,2-b]pyridazin-3-ylethynyl)pyridin-2-yl)-5-phenyl-4,5-dihydro-1H-pyrazole-1-carboxamide 2,2,2-trifluoroacetate;

(53) (3-(imidazo[1,2-b]pyridazin-3-ylethynyl)phenyl)(5-phenyl-4,5-dihydro-1H-pyrazol-1-yl)methanone 2,2,2-trifluoroacetate;

(54) (3-(imidazo[1,2-b]pyridazin-3-ylethynyl)-4-methylphenyl)(5-phenyl-4,5-dihydro-1H-pyrazol-1-yl)methanone 2,2,2-trifluoroacetate;

(55) N-(3-(imidazo[1,2-a]pyridin-3-ylethynyl)phenyl)-5-phenyl-4,5-dihydro-1H-pyrazole-1-carboxamide;

(56) 5-phenyl-N-(3-(pyrazolo[1,5-a]pyrimidin-3-ylethynyl)phenyl)-4,5-dihydro-1H--pyrazole-1-carboxamide;

(57) N-(3-(imidazo[1,2-a]pyrazin-3-ylethynyl)phenyl)-5-phenyl-4,5-dihydro-1H-pyrazole-1-carboxamide;

(58) N-(3-(imidazo[1,2-a]pyrimidin-3-ylethynyl)phenyl)-5-phenyl-4,5-dihydro-1H-pyrazole-1-carboxamide;

(59) N-(3-((8-aminoimidazo[1,2-a]pyridin-3-yl)ethynyl)phenyl)-5-phenyl-4,5-dihydro-1H-pyrazole-1-carboxamide hydrochloride;

(60) N-(3-((8-acetamidoimidazo[1,2-a]pyridin-3-yl)ethynyl)phenyl)-5-phenyl-4,5-dihydro-1H-pyrazole-1-carboxamlde;

(61) N-(3-((8-(3-methylureido)imidazo[1,2-a]pyridin-3-yl)ethynyl)phenyl)-5-phenyl-4,5-dihydro-1H-pyrazole-1-carboxamide 2,2,2-trifluoroacetate;

(62) N-(3-((8-(cyclopropanecarboxamido)imidazo[1,2-a]pyridin-3-yl)ethynyl)phenyl)-5-phenyl-4,5-dihydro-1H-pyrazole-1-carboxamide 2,2,2-trifluoroacetate;

(63) N-(3-((8-(cyclobutanecarboxamido)imidazo[1,2-a]pyridin-3-yl)ethynyl)phenyl)-5-phenyl-4,5-dihydro-1H-pyrazole-1-carboxamide 2,2,2-trifluoroacetate;

(64) methyl (3-((3-(5-phenyl-4,5-dihydro-1H-pyrazole-1-carboxamido)phenyl)ethynyl)imidazo[1,2-a]pyridin-8-yl)carbamate;

(65) N-(3-((8-((4-(methylsulfonyl)phenyl)amino)imidazo[1,2-a]pyridin-3-yl)ethynyl)phenyl)-5-phenyl-4,5-dihydro-1H-pyrazole-1-carboxamide 2,2,2-trifluoroacetate;

(66) N-(3-((8-((5-methyl-1H-pyrazol-3-yl)amino)imidazo[1,2-a]pyridin-3-yl)ethynyl)phenyl)-5-phenyl-4,5-dihydro-1H-pyrazole-1-carboxamide 2,2,2-trifluoroacetate;

(67) N-(3-((8-((1-methyl-1H-pyrazol-4-yl)amino)imidazo[1,2-a]pyridin-3-yl)ethynyl)phenyl)-5-phenyl-4,5-dihydro-1H-pyrazole-1-carboxamide 2,2,2-trifluoroacetate; and

(68) N-(3-((8-((1-methyl-1H-pyrazol-3-yl)amino)imidazo[1,2-a]pyridin-3-yl)ethynyl)phenyl)-5-phenyl-4,5-dihydro-1H-pyrazole-1-carboxamide 2,2,2-trifluoroacetate.

The compound represented by formula 1 of the present invention can be used as a form of a pharmaceutically acceptable salt, in which the salt is preferably acid addition salt formed by pharmaceutically acceptable free acids. The acid addition salt herein can be obtained from inorganic acids such as hydrochloric acid, nitric acid, phosphoric acid, sulfuric acid, hydrobromic acid, hydroiodic acid, nitrous acid, and phosphorous acid; non-toxic organic acids such as aliphatic mono/dicarboxylate, phenyl-substituted alkanoate, hydroxy alkanoate, alkandioate, aromatic acids, and aliphatic/aromatic sulfonic acids; or organic acids such as acetic acid, benzoic acid, citric acid, lactic acid, maleic acid, gluconic acid, methanesulfonic acid, 4-toluenesulfonic acid, tartaric acid, and fumaric acid. The pharmaceutically non-toxic salts are exemplified by sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, suberate, cabacate, fumarate, maliate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, hydroxybutylate, glycolate, malate, tartrate, methanesulfonate, propanesulfonate, naphthalene-1-sulfonate, naphthalene-2-sulfonate, and mandelate.

The acid addition salt according to the present invention can be prepared by the conventional method known to those in the art. For example, the derivative represented by formula 1 is dissolved in an organic solvent such as methanol, ethanol, acetone, dichloromethane, and acetonitrile, to which organic acid or inorganic acid is added to induce precipitation. Then, the precipitate is filtered and dried to give the salt. Or the solvent and the excessive acid are distillated under reduced pressure, and dried to give the salt. Or the precipitate is crystallized in an organic solvent to give the same.

A pharmaceutically acceptable metal salt can be prepared by using a base. Alkali metal or alkali earth metal salt is obtained by the following processes: dissolving the compound in excessive alkali metal hydroxide or alkali earth metal hydroxide solution; filtering non-soluble compound salt; evaporating the remaining solution and drying thereof. At this time, the metal salt is preferably prepared in the pharmaceutically suitable form of sodium, potassium, or calcium salt. And the corresponding silver salt is prepared by the reaction of alkali metal or alkali earth metal salt with proper silver salt (ex; silver nitrate).

Furthermore, the present invention includes not only the compound represented by formula 1 and the pharmaceutically acceptable salt thereof, but also solvates, optical isomers, hydrates, etc., which may be prepared therefrom.

In one aspect of the present invention, the compound can be prepared by a method comprising the following steps, as shown in reaction formula 1 below:

preparing a compound represented by formula 4 by first reacting a compound represented by formula 2 with DSC (N,N-disuccinimidyl carbonate) to introduce a carbonyl group to an amine group, and then reacting thereof with a compound represented by formula 3 (step 1); and

preparing a compound represented by formula 1-1 by reacting the prepared compound represented by formula 4 with a compound represented by Formula 5 (step 2).

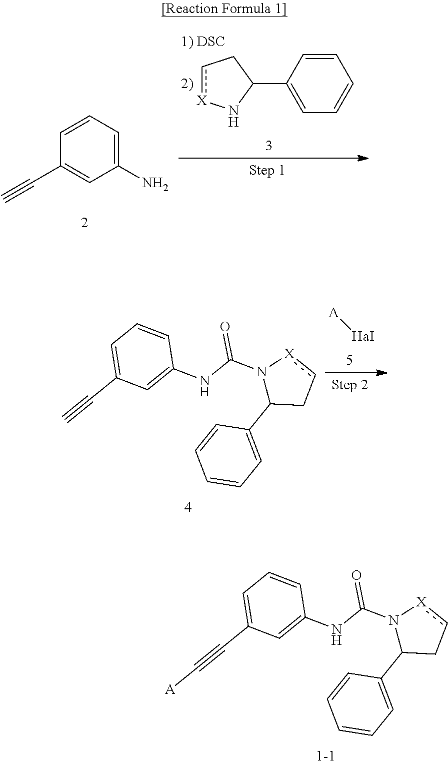

(In reaction formula 1,

the compound represented by formula 1-1 is a compound included in the compound of formula 1 above;

A is as defined in formula 1 above; and

Hal is halogen).

Hereinafter, the preparation method of the compound represented by formula 1-1 is described in detail step by step.

In the preparation method of the compound represented by formula 1-1, step 1 is a step of preparing a compound represented by formula 4 by first reacting a compound represented by formula 2 with DSC (N,N-disuccinimidyl carbonate) to introduce a carbonyl group to an amine group, and then reacting thereof with a compound represented by formula 3. The reaction of step 1 can be performed in the presence of a base such as DIEA (N,N-diisopropylethylamine). Although there is no particular limitation on the type of the reaction solvent, acetonitrile or the like can be preferably used, and the reaction can be carried out in the range of −20° C. to 40° C.

In the preparation method of the compound represented by formula 1-1, step 2 is a step of preparing a compound represented by formula 1-1 by reacting the prepared compound represented by formula 4 with a compound represented by Formula 5. Although there is no particular limitation on the type of the reaction solvent used in step 2, dimethylformamide or the like can be preferably used. The reaction temperature can be adjusted in the range of 50° C. to 90° C., and the reaction can be performed for 4 to 8 hours, but not always limited thereto.

In one aspect of the present invention, the compound can be prepared by a method comprising the following steps, as shown in reaction formula 2 below:

preparing a compound represented by formula 3 from a compound represented by the formula 2 (step 1);

preparing a compound represented by formula 4 by removing the protecting group of the compound represented by formula 3 prepared in step 1 (step 2);

preparing a compound represented by formula 6 by reacting the compound represented by formula 4 prepared in step 2 with a compound represented by formula 5 (step 3); and

preparing a compound represented by formula 1-2 by introducing the substituent R¹ to the amine group of the compound represented by formula 6 prepared in step 3 (step 4).

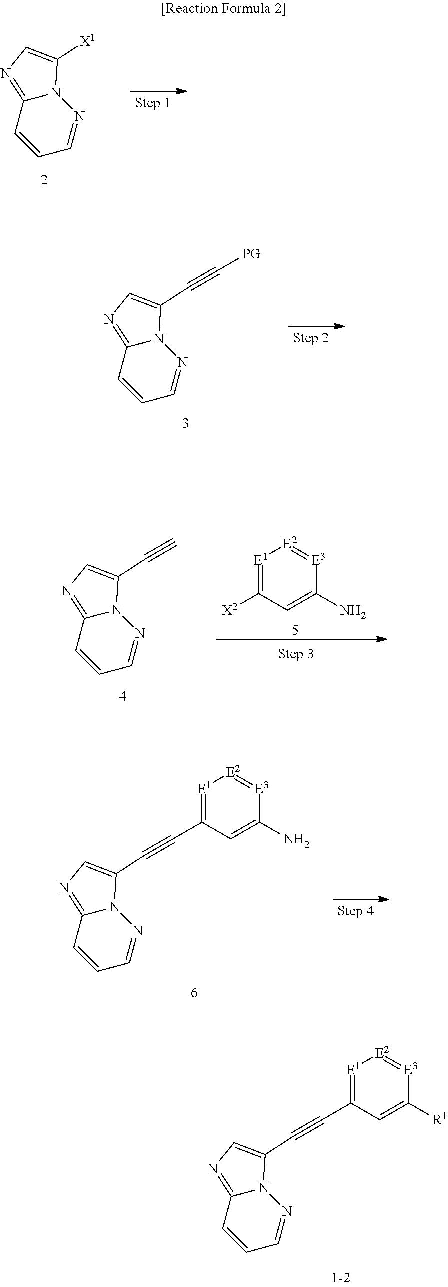

(In reaction formula 2,

the compound represented by formula 1-2 is a compound included in the compound of formula 1 above;

X¹ and X² are independently halogen;

PG is protecting group (PG); and

E¹, E², E³ and R¹ are independently as defined in formula 1 above).

In another aspect, X¹ and X² can be independently selected from the group consisting of —F, —CI, —Br and —I; the PG can be used without limitation as long as it is a known protecting group, and one specific example thereof can be trimethylsilyl (TMS).

Hereinafter, the preparation method of the compound represented by formula 1-2 is described in detail step by step.

In the preparation method of the compound represented by formula 1-2, step 1 is a step of preparing a compound represented by formula 3 from a compound represented by the formula 2. Particularly, step 2 is a step of preparing a compound represented by formula 3 by substituting acetylene in which a protecting group is substituted at the terminal, specifically trimethylsilylacetylene, at the position where X¹ of a compound represented by formula 2 is bonded. Although there is no particular limitation on the type of the reaction solvent, acetonitrile or the like can be preferably used, and the reaction can be carried out in the range of 70° C. to 90° C.

In the preparation method of the compound represented by formula 1-2, step 2 is a step of preparing a compound represented by formula 4 by removing the protecting group of the compound represented by formula 3 prepared in step 1. The removal of the protecting group can be performed by applying a known method as a method of removing the protecting group without limitation, depending on the type of the introduced protecting group. If the protecting group introduced as one specific example is trimethylsilyl (TMS), trimethylsilyl (TMS) can be removed by treating potassium carbonate in a methanol solvent.

In the preparation method of the compound represented by formula 1-2, step 3 is a step of preparing a compound represented by formula 6 by reacting the compound represented by formula 4 prepared in step 2 with a compound represented by formula 5. At this time, the reaction can be performed in the presence of ethyl acetate in the temperature range of 30° C. to 70° C. The reaction can be performed for 1 to 3 hours, but not always limited thereto.

In the preparation method of the compound represented by formula 1-2, step 4 is a step of preparing a compound represented by formula 1-2 by introducing the substituent to the amine group of the compound represented by formula 6 prepared in step 3. By performing step 4, the compound provided in one aspect of the present invention can be prepared.

In another aspect of the present invention, the present invention provides a pharmaceutical composition comprising the compound represented by formula 1, the stereoisomer thereof, the hydrate thereof, or the pharmaceutically acceptable salt thereof as an active ingredient for the prevention or treatment of kinase-related disease.

At this time, the kinase can be one or more kinases selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK.

In one aspect, the kinase-related disease can be one or more diseases selected from the group consisting of diseases/disorders that can be at least partially regulated by programmed necrosis, apoptosis, or production of inflammatory cytokines, especially inflammatory bowel disease (including Crohn's disease and ulcerative colitis), psoriasis, retinal detachment (and degeneration), retinitis pigmentosa, macular degeneration, pancreatitis, atopic dermatitis, arthritis (including rheumatoid arthritis, spondylarthritis, gout, juvenile idiopathic arthritis (systemic onset juvenile idiopathic arthritis (SoJIA)) and psoriatic arthritis), systemic lupus erythematosus (SLE), Sjogren's syndrome, systemic scleroderma, anti-phospholipid syndrome (APS), vasculitis, osteoarthritis, liver injury/disease (non-alcoholic steatohepatitis, alcoholic steatohepatitis, autoimmune hepatitis, autoimmune hepatobiliary disease, primary sclerosing cholangitis (PSC), acetaminophen toxicity, hepatotoxicity), kidney injury/trouble (nephritis, kidney transplantation, surgery, administration of nephrotoxic drugs such as cisplatin, acute kidney injury (AKI)), celiac disease, autoimmune idiopathic thrombocytopenia purpura (autoimmune ITP), transplant rejection (rejection of transplanted organs, tissues and cells), ischemic reperfusion injury of parenchymal organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), neonatal hypoxic brain injury, ischemic brain injury, traumatic brain injury, allergic disease (including asthma and atopic dermatitis), burns, multiple sclerosis, type I diabetes, Wegener's granulomatosis, pulmonary sarcoidosis, Behcet's disease, interleukin-1 converting enzyme (ICE, also known as caspase-1) associated fever syndrome, chronic obstructive pulmonary disease (COPD), tobacco smoke-induced injury, cystic fibrosis, tumor necrosis factor receptor-associated periodic syndrome (TRAPS), neoplastic tumor, periodontitis, NEMO-mutation (mutation of NF-kappa-B essential modulator gene (also known as IKK gamma or IKKG)), in particular, NEMO-deficiency syndrome, HOIL-1 deficiency ((also known as RBCK1) heme-oxidized IRP2 ubiquitin ligase-1 deficiency), linear ubiquitin chain assembly complex (LUBAC) deficiency syndrome, blood and parenchymal organ malignant tumor, bacterial infection and viral infection (such as influenza, staphylococcus and mycobacterium (tuberculosis)), lysosome accumulation disease (including Gaucher's disease, GM2 gangliosidosis, alpha-mannoside accumulation, aspartylglucosamineuria, cholesteryl ester accumulation disease, chronic hexosaminidase A deficiency, cystine accumulation, Danon's disease, Fabry's disease, Faber's disease, fucosidosis, galactosialic acidosis, GM1 gangliosidosis, mucolipidosis, infantile free sialic acid accumulation disease, hexosaminidase A deficiency in children, Krabe's disease, lysosomal acid lipase deficiency, metachromatic leukodystrophy, mucopolysaccharide disorder, multiple sulfatase deficiency disease, Niemann-Pick's disease, neuroserobic lipofuscinosis, Pompe's disease, pyknodysostosis, Sandhof's disease, Schindler's disease, sialic acid accumulation disease, Tay-Sachs disease and Wolman's disease), Stevens-Johnson syndrome, glaucoma, spinal cord injury, pancreatic duct adenocarcinoma, hepatocellular carcinoma, mesothelioma, melanoma, acute liver failure, and the like.

The compound represented by formula 1 or the pharmaceutically acceptable salt thereof can be administered orally or parenterally and be used in general forms of pharmaceutical formulation. That is, the compound represented by formula 1 or the pharmaceutically acceptable salt thereof can be prepared for oral or parenteral administration by mixing with generally used diluents or excipients such as fillers, extenders, binders, wetting agents, disintegrating agents and surfactants. Solid formulations for oral administration are tablets, pills, powders, granules and capsules. These solid formulations are prepared by mixing the said betaine with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration can comprise sterilized aqueous solutions, water-insoluble excipients, suspensions, emulsions. Water insoluble excipients and suspensions can contain, in addition to the active compound or compounds, propylene glycol, polyethylene glycol, vegetable oil like olive oil, injectable ester like ethylolate, etc.

The pharmaceutical composition comprising the compound represented by formula 1 or the pharmaceutically acceptable salt thereof as an active ingredient can be administered by parenterally and the parenteral administration includes subcutaneous injection, intravenous injection, intramuscular injection, or intrathoracic injection.

To prepare the compound represented by formula I or the pharmaceutically acceptable salt thereof as a formulation for parenteral administration, the compound represented by formula 1 or the pharmaceutically acceptable salt thereof is mixed with a stabilizer or a buffering agent in water to produce a solution or suspension, which is then formulated as ampoules or vials. The composition herein can be sterilized and additionally contains preservatives, stabilizers, wettable powders or emulsifiers, salts and/or buffers for the regulation of osmotic pressure, and other therapeutically useful materials, and the composition can be formulated by the conventional mixing, granulating or coating method.

The formulations for oral administration are exemplified by tablets, pills, hard/soft capsules, solutions, suspensions, emulsions, syrups, granules, elixirs, and troches, etc. These formulations can include diluents (for example, lactose, dextrose, sucrose, mannitol, sorbitol, cellulose, and/or glycine) and lubricants (for example, silica, talc, stearate and its magnesium or calcium salt, and/or polyethylene glycol) in addition to the active ingredient. Tablets can include binding agents such as magnesium aluminum silicate, starch paste, gelatin, methylcellulose, sodium carboxymethylcellulose and/or polyvinylpyrolidone, and if necessary disintegrating agents such as starch, agarose, alginic acid or its sodium salt or azeotropic mixtures and/or absorbents, coloring agents, flavours, and sweeteners can be additionally included thereto.

The pharmaceutical composition can be administered as an individual therapeutic agent or can be used in combination with other anticancer agents in use.

In another aspect of the present invention, the present invention provides a health functional food composition comprising the compound represented by formula 1, the stereoisomer thereof, the hydrate thereof, or the pharmaceutically acceptable salt thereof as an active ingredient for the prevention or amelioration of kinase-related disease.

At this time, the kinase can be one or more kinases selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK.

Since the kinase-related disease is the same as described above, detailed descriptions are omitted to avoid redundant descriptions.

The compound represented by formula 1 of the present invention can be used as a food additive. In that case, the compound represented by formula 1 of the present invention can be added as it is or as mixed with other food components according to the conventional method. The mixing ratio of active ingredients can be regulated according to the purpose of use (prevention or amelioration). In general, the compound of the present invention is preferably added to food or beverages by 0.1˜90 weight part for the total weight of the food or beverages. However, if long term administration is required for health and hygiene or regulating health condition, the content can be lower than the above but higher content can be accepted as well since the compound of the present invention has been proved to be very safe.

The health beverage composition of the present invention can additionally include various flavors or natural carbohydrates, etc, like other beverages. The natural carbohydrates above can be one of monosaccharides such as glucose and fructose; disaccharides such as maltose and sucrose; polysaccharides such as dextrin and cyclodextrin, and sugar alcohols such as xilytole, sorbitol and erythritol. Besides, natural sweetening agents (thaumatin, stevia extract, for example rebaudioside A, glycyrrhizin, etc.) and synthetic sweetening agents (saccharin, aspartame, etc.) can be included as a sweetening agent. The content of the natural carbohydrate is preferably 1˜20 g and more preferably 5˜12 g in 100 g of the composition of the invention.

In addition to the ingredients mentioned above, the compound represented by formula 1 of the present invention can include in variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The compound represented by formula 1 of the present invention can also include natural fruit juice, fruit beverages and fruit flesh addable to vegetable beverages.

In another aspect of the present invention, the present invention provides a method for treating kinase-related disease comprising a step of administering the compound represented by formula 1, the stereoisomer thereof, the hydrate thereof, or the pharmaceutically acceptable salt thereof to a subject in need thereof.

In another aspect of the present invention, the present invention provides the compound represented by formula 1, the stereoisomer thereof, the hydrate thereof, or the pharmaceutically acceptable salt thereof for use in the prevention or treatment of kinase-related disease.

In another aspect of the present invention, the present invention provides a use of the compound represented by formula 1, the stereoisomer thereof, the hydrate thereof, or the pharmaceutically acceptable salt thereof for preparing a medicament for use in the prevention or treatment of kinase-related disease.

Since the N-(3-(imidazo[1,2-b]pyridazin-3-ylethynyl)-4-methylphenyl)-5-phenyl-4,5-dihydro-1H-pyrazole-1-carboxamide derivative provided in one aspect of the present invention exhibits excellent inhibitory activity against at least one kinase selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK, there is an effect that can be used as a therapeutic agent for kinase-related disease, which is supported by examples and experimental examples described below.

Hereinafter, the present invention will be described in detail by the following examples and experimental examples.

However, the following examples and experimental examples are only for illustrating the present invention, and the contents of the present invention are not limited thereto.

The compounds synthesized in examples of the present invention were purified under the following HPLC conditions or subjected to structural analysis.

For preparative medium pressure liquid chromatography (MPLC), CombiFlash Rf +UV of TELEEDYNE ISCO was used.

Conditions of Analytical HPLC (ACQUITY UPLC H-Class Core System)

A UPLC system (ACQUITY UPLC PDA Detector, Waters) equipped with a mass QDA Detector (Waters) was used. ACQUITY UPLC®BEH C18 (1.7 μm, 2.1×50 mm, Waters) was used as a column, and chromatography was performed at the column temperature of 30° C.

Water containing 0.1% formic acid was used as moving phase A, and acetonitrile containing 0.1% formic acid was used as moving phase B.

Gradient condition (3 minutes with 10-100% B, movement rate=0.6 ml/min).

Prep-LCMS (Preparative-Liquid Chromatography Mass Spectrometry) for Purification

An autopurification HPLC system (2767 sample manger, 2545 binary gradient module, 2998 Photodiode Array Detector, Waters) equipped with a mass QDA Detector (Waters) was used. SunFire®Prep C18 OBD™ (5 μm, 19×50 mm, Waters) was used as a column, and chromatography was performed at room temperature.

Water containing 0.035% trifluoroacetic acid was used as moving phase A, and methanol containing 0.035% trifluoroacetic acid was used as moving phase B.

Gradient condition (10 minutes with 15-100% B, movement rate=25 ml/min).

Prep-150 LC System (Preparative-Liquid Chromatography UV Spectrometry) for Purification

A Prep 150 LC system (2545 Quaternary gradient module, 2998 Photodiode Array Detector, Fraction collector III, Waters) was used. XTERRA®Prep RP18 OBD™ (10 μm, 30×300 mm, Waters) was used as a column, and chromatography was performed at room temperature.

Gradient condition (120 minutes with 3-100% B, movement rate=40 ml/min).

Conditions of HPLC for Chiral Compound Separation

A Prep 150 LC system (2545 Quaternary gradient module, 2998 Photodiode Array Detector, Fraction collector III, Waters) was used. CHIRALPAK®IB (5 μm, 20×250 mm, DAICEL) was used as a column, and chromatography was performed at room temperature.

N-hexane was used as moving phase A and ethanol was used as moving phase B.

Moving phase condition (40 minutes with 40% B movement rate=20 ml/min).

The commercial reagents used herein were used without further purification. Room temperature in the present invention refers to a temperature of about 20 to 25° C. Concentration under reduced pressure or distillation of solvent was performed using a rotary evaporator.

### PREPARATIVE EXAMPLE 1

### Preparation of 5-phenyl-4,5-dihydro-1H-pyrazole

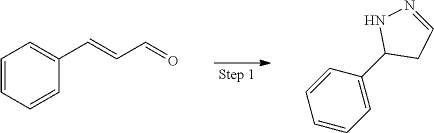 [SMILES: O=C/C=C/c1ccccc1 ; C1=NNC(c2ccccc2)C1]

Hydrazine monohydrate (865.98 g, 26.48 mol, 977.40 mL) was dissolved in t-BuOH (500 mL) and heated at 110° C. (E)-cinnamic aldehyde (500 g, 3.78 mol, 476.19 mL) was added thereto, followed by stirring for hours. Upon completion of the reaction, the temperature was lowered to room temperature, and the reaction mixture was diluted with water (1.5 L), followed by extraction with DCM (500 mL*4). The combined organic layer was extracted with brine (500 mL), dried over sodium sulfate, and concentrated to give 5-phenyl-4,5-dihydro-1H-pyrazole (500 g, crude) as a liquid, which was used in the next reaction without purification.

MS (m/z): 147.1 [M+1]⁺, UPLC r. t. (min): 0.98

¹H NMR (400 MHz, CDCl₃) δ 7.43-7.30 (m, 5H), 4.74 (m, 1H), 3.16 (m, 1H), 2.73 (m, 1H).

### PREPARATIVE EXAMPLE 2

### Preparation of 5-(3-chlorophenyl)-4,5-dihydro-1H-pyrazole

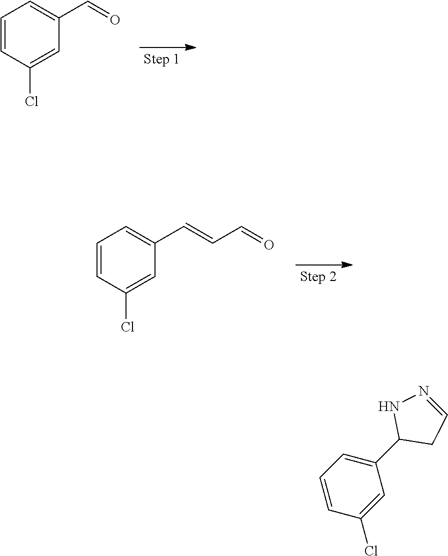 [SMILES: O=Cc1cccc(Cl)c1 ; O=C/C=C/c1cccc(Cl)c1 ; Clc1cccc(C2CC=NN2)c1]

### Step Preparation of (E)-3-(3-chlorophenyl)acrylaldehyde

3-Chlorobenzaldehyde (8.06 mL, 71.14 mmol) and 2-(triphenylphosphanylidene)acetaldehyde (21.65 g, 71.14 mmol) were dissolved in DCM (300 mL), followed by stirring at 40° C. for 20 hours. After confirming the completion of the reaction by TLC (PE:EA=10:1), the reaction mixture was concentrated under reduced pressure and purified by medium pressure liquid chromatography (hexane/ethylacetate) to give (E)-3-(3-chlorophenyl)acrylaldehyde (5.5 g, 46.4%) as a pale yellow solid.

¹H NMR (400 MHz, CDCl₃) δ 9.61 (s, 1H), 7.47-7.41 (m, 1H), 7.37-7.24 (m, 4H), 6.60 (dd, J=7.6, 16.0 Hz, 1H)

### Step 2: Preparation of 5-(3-chlorophenyl)-4,5-dihydro-1H-pyrazole

(E)-3-(3-chlorophenyl)prop-2-enal (4 g, 24.01 mmol) was dissolved in t-BuOH (4 mL), to which hydrazine (12 mL, 241.96 mmol) was added, followed by stirring at 90° C. for 2 hours. Upon completion of the reaction, the temperature was lowered to room temperature, and the reaction mixture was washed with water (80 mL*3) and brine (60 mL). The organic layer was dried over sodium sulfate and concentrated to give 5-(3-chlorophenyl)-4,5-dihydro-1H-pyrazole (4 g, crude) as a liquid, which was used in the next reaction without purification.

MS: m/z 181.5 [M+H]⁺

¹H NMR (400 MHz, CDCl₃) δ 7.24 (m, 2H), 7.21-7.17 (m, 2H), 6.80-6.77 (m, 1H), 4.67 (m, 1H), 3.11 (m, 1H), 2.64 (m, 1H)

The compounds of Preparative Examples 3 to 11 were prepared by the similar manner to the methods described in Preparative Examples 1 to 2. The compound names, chemical formulas, and UPLC analysis results of the compounds of Preparative Examples 3 to 11 are shown below and were used in the preparation of the compounds of the following examples.

### PREPARATIVE EXAMPLE 3

### 5-(4-chlorophenyl)-4,5-dihydro-1H-pyrazole

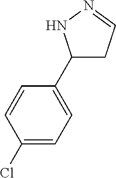 [SMILES: Clc1ccc(C2CC=NN2)cc1]

MS (m/z): 181.1 [M+1]⁺, UPLC r. t. (min): 1.31

### PREPARATIVE EXAMPLE 4

### 5-(3,5-difluorophenyl)-4,5-dihydro-1H-pyrazole

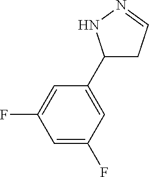 [SMILES: Fc1cc(F)cc(C2CC=NN2)c1]

MS (m/z): 183.1 [M+1]⁺, UPLC r. t. (min): 1.02

### PREPARATIVE EXAMPLE 5

### 5-(4-(trifluoromethyl) phenyl)-4,5-dihydro-1H-pyrazole

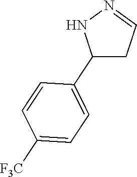 [SMILES: FC(F)(F)c1ccc(C2CC=NN2)cc1]

MS (m/z): 215.1 [M+1]⁺, UPLC r. t. (min): 1.30

### PREPARATIVE EXAMPLE 6

### 5-(3-fluoropheny)-4,5-dihydro-1H-pyrazole

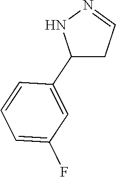 [SMILES: Fc1cccc(C2CC=NN2)c1]

MS (m/z): 165.1 [M+1]⁺, UPLC r. t. (min): 1.09

### PREPARATIVE EXAMPLE 7

### 5-(4-fluorophenyl)-4,5-dihydro-1H-pyrazole

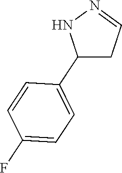 [SMILES: Fc1ccc(C2CC=NN2)cc1]

MS (m/z): 165.1 [M+1]⁺, UPLC r. t. (min): 1.09

### PREPARATIVE EXAMPLE 8

### 3-(4,5-dihydro-1H-pyrazol-5-yl)pyridine

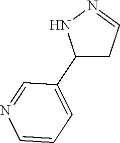 [SMILES: C1=NNC(c2cccnc2)C1]

MS (m/z): 148.1 [M+1]⁺, UPLC r. t. (min): 0.23

### PREPARATIVE EXAMPLE 9

### 2-(4,5-dihydro-1H-pyrazol-5-yl)pyridine

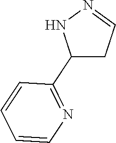 [SMILES: C1=NNC(c2ccccn2)C1]

MS (m/z): 148.1 [M+1]⁺, UPLC r. t. (min): 0.23

### PREPARATIVE EXAMPLE 10

### 4-(4,5-dihydro-1H-pyrazol-5-yl)pyridine

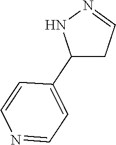 [SMILES: C1=NNC(c2ccncc2)C1]

MS (m/z): 148.1 [M+1]⁺, UPLC r. t. (min): 0.23

### PREPARATIVE EXAMPLE 11

### 5-(4-methoxyphenyl)-4,5-dihydro-1H-pyrazole

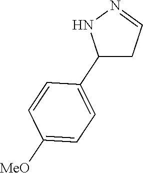 [SMILES: COc1ccc(C2CC=NN2)cc1]

MS (m/z): 177.2 [M+1]⁺, UPLC r. t. (min): 0.96

### EXAMPLE 1

### Preparation of N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate

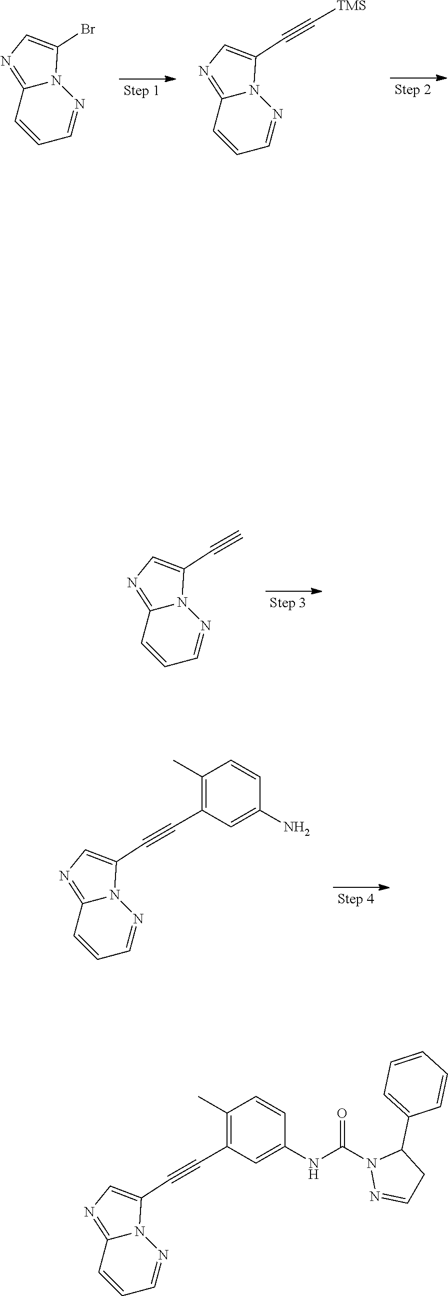 [SMILES: Brc1cnc2cccnn12 ; C[Si](C)(C)C#Cc1cnc2cccnn12 ; C#Cc1cnc2cccnn12 ; Cc1ccc(N)cc1C#Cc1cnc2cccnn12 ; Cc1ccc(NC(=O)N2N=CCC2c2ccccc2)cc1C#Cc1cnc2cccnn12]

### Step 1: Preparation of 3-((trimethylsilyl)ethynyl)imidazo[1,2-b]pyridazine

3-Bromoimidazo[1,2-b]pyridazine (10 g, 50.5 mmol), Pd(PPh₃)₄ (2.92 g, 2.52 mmol) and CuI (0.962 g, 5.05 mmol) were added. The mixture was diluted by adding acetonitrile (50.5 ml). The gas was removed by ultrasonic treatment for 5 minutes while blowing nitrogen. Trimethylsilylacetylene (7.44 g, 76 mmol) and triethylamine (28.2 ml, 202 mmol) were added to the reaction mixture, followed by reaction at 80° C. for 1 hour. Upon completion of the reaction, the reaction mixture was filtered with celite. The obtained filtrate was concentrated under reduced pressure using a rotary evaporator to give 3-((trimethylsilyl)ethynyl)imidazo[1,2-b]pyridazine (11 g, 101%), which was used in the next reaction without purification.

MS (m/z): 216.15 [M+1]⁺, UPLC r. t. (min): 1.63

### Step 2: Preparation of 3-ethynylimidazo[1,2-b]pyridazine

The compound 3-((trimethylsilyl)ethynyl)imidazo[1,2-b]pyridazine (11 g, 51.1 mmol) obtained in step 1 of Example 1 and potassium carbonate (21.18 g, 153 mmol) were dissolved in methanol (51.1 ml), followed by stirring for 1 hour. Upon completion of the reaction, the reaction mixture was filtered, and the obtained filtrate was concentrated under reduced pressure using a rotary evaporator. The concentrate was purified by medium pressure liquid chromatography (dichloromethane/ethyl acetate) to give the target compound 3-ethynylimidazo[1,2-b]pyridazine (5.78 g, 80%) as a solid.

MS (m/z): 144.03 [M+1]⁺, UPLC r. t. (min): 0.87

¹H NMR (400 MHz, DMSO-d₆) δ 8.66 (dd, J=4.4, 1.5 Hz, 3H), 7.70-7.61 (m, 6H), 7.59-7.51 (m, 5H), 4.96 (s, 2H).

### Step 3: Preparation of 3-(imidazo[1,2-b]pyridazin-3-ylethynyl)-4-methylaniline

The compound 3-ethynylimidazo[1,2-b]pyridazine (400 mg, 2.79 mmol) obtained in step 2 of Example 1 and 3-iodo-4-methylaniline (715 mg, 3.07 mmol) were dissolved in ethyl acetate (9.3 ml), and the gas was removed by ultrasonic treatment for 5 minutes while blowing nitrogen. Pd(PPh₃)₄ (161 mg, 0.140 mmol), CuI (53.2 mg, 0.279 mmol) and DIPEA (976 μl, 5.59 mmol) were added to the reaction mixture, followed by stirring at 50° C. for 2 hours. The reaction mixture was filtered with celite and washed with ethyl acetate. The obtained filtrate was concentrated under reduced pressure, and then purified by medium pressure liquid chromatography (dichloromethane/ethyl acetate) to give the target compound 3-(imidazo[1,2-b]pyridazin-3-ylethynyl)-4-methylaniline (555 mg, 80%) as a solid.

MS (m/z): 249.16 [M+1]⁺, UPLC r. t. (min): 1.12

¹H NMR (400 MHz, DMSO-d₆) δ 8.69 (dd, J=4.4, 1.2 Hz, 1H), 8.29-8.07 (m, 2H), 7.36 (dd, J=9.2, 4.4 Hz, 1H), 6.99 (d, J=8.2 Hz, 1H), 6.79 (d, J=2.4 Hz, 1H), 6.59 (dd, J=8.2, 2.4 Hz, 1H), 5.08 (s, 2H), 2.35 (s, 3H).

### Step 4: Preparation of N-(3-(imidazo[1,2-b]pyridazin-3-ylethynyl)-4-methylphenyl)-5-phenyl-4,5-dihydro-1H-pyrazole-1-carboxamide trifluoroacetate

The compound obtained in step 3 of Example 1 (150 mg, 0.604 mmol) and pyridine (0.12 ml, 1.51 mmol) were dissolved in dichloromethane (2 ml), to which 1,1′-carbonyldiimidazole (127 mg, 0.785 mmol) was added at 0° C., followed by stirring at 0° C. for 3 hours. The reaction mixture was concentrated under reduced pressure using a rotary evaporator, and dissolved in tetrahydrofuran (2 ml), to which triethylamine (0.21 ml, 1.51 mmol) and 5-phenyl-4,5-dihydro-1H-pyrazole (0.12 ml, 0.906 mmol) were slowly added dropwise, followed by stirring at 60° C. for 3 hours. Upon completion of the reaction, the reactant was concentrated under reduced pressure using a rotary evaporator, extracted with ethyl acetate and brine, and the organic layers were combined. The organic layer was dried over sodium sulfate, concentrated under reduced pressure, and purified using a Prep-150 device to give the target compound N-(3-(imidazo[1,2-b]pyridazin-3-ylethynyl)-4-methylphenyl)-5-phenyl-4,5-dihydro-1H-pyrazole-1-carboxamide trifluoroacetate (178 mg, 70%).

MS (m/z): 421.3 [M+1]⁺, UPLC r. t. (min): 1.71

### EXAMPLE 2

### Preparation of (S)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate; and

### EXAMPLE 3

### (R)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate

The compound of Example 1 prepared in the above step was separated into (S)—N-(3-(imidazo[1,2-b]pyridazin-3-ylethynyl)-4-methylphenyl)-5-phenyl-4,5-dihydro-1H-pyrazole-1-carboxamide trifluoroacetate (Example 2) and (R)-—N-(3-(imidazo[1,2-b]pyridazin-3-ylethynyl)-4-methylphenyl)-5-phenyl-4,5-dihydro-1H-pyrazole-1-carboxamide trifluoroacetate (Example 3) using supercritical fluid chromatography (chiralcel OD-3 50×4.6 mm, 40% MeON containing 0.05% DEA in CO₂, 3 mL/min).

The compounds of Examples 4 to 43 were prepared by the similar manner to the methods described in Examples 1 to 3. The compound names, chemical formulas, and UPLC/NMR analysis results of the compounds of Examples 1 to 43 are summarized in Table 1 below.

TABLE 1

| | | | UPLC | |
|---|---|---|---|---|
| | | | [M + 1]⁺; | |
| Example | Structure | Compound Name | r.t. (min) | ¹H NMR |
| 1 | 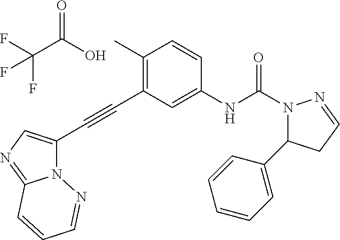 | N-(3-(imidazo[1,2- b]pyridazin-3- ylethynyl)-4- methylphenyl)-5-phenyl- 4,5-dihydro-1H- pyrazole-1-carboxamide 2,2,2-trifluoroacetate | 421.3; 1.71 | ¹H NMR (400 MHz, DMSO-d₆) δ 9.17 (s, 1H), 8.70 (dd, J = 4.4, 1.6 Hz, 1H), 8.23 (dd, J = 9.2, 1.6 Hz, 1H), 8.19 (s, 1H), 7.90 (d, J = 2.3 Hz, 1H), 7.50 (m, 1H), 7.35 (m, 4H), 7.20 (m, 5H), 5.31 (m, 1H), 3.55 (m, 1H), 2.75 (ddd, J = 18.7, 5.7, 1.8 Hz, 1H), 2.43 (s, 3H). |
| 2 | 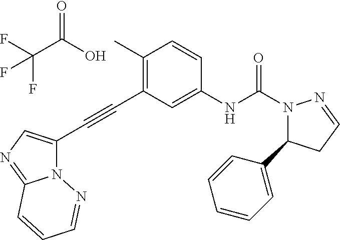 | (S)-N-(3-(imidazo[1,2- b]pyridazin-3- ylethynyl)-4- methylphenyl)-5-phenyl- 4,5-dihydro-1H- pyrazole-1-carboxamide 2,2,2-trifluoroacetate | 420.17; 1.71 | ¹H NMR (400 MHz, DMSO-d₆) δ 9.17 (s, 1H), 8.70 (dd, J = 4.4, 1.6 Hz, 1H), 8.23 (dd, J = 9.2, 1.6 Hz, 1H), 8.19 (s, 1H), 7.90 (d, J = 2.3 Hz, 1H), 7.50 (m, 1H), 7.35 (m, 4H), 7.20 (m, 5H), 5.31 (m, 1H), 3.55 (m, 1H), 2.75 (ddd, J = 18.7, 5.7, 1.8 Hz, 1H), 2.43 (s, 3H). |
| 3 | 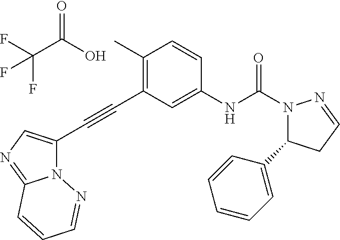 | (R)-N-(3-(imidazo[1,2- b]pyridazin-3- ylethynyl)-4- methylphenyl)-5-phenyl- 4,5-dihydro-1H- pyrazole-1-carboxamide 2,2,2-trifluoroacetate | 420.17; 1.71 | ¹H NMR (400 MHz, DMSO-d₆) δ 9.17 (s, 1H), 8.70 (dd, J = 4.4, 1.6 Hz, 1H), 8.23 (dd, J = 9.2, 1.6 Hz, 1H), 8.19 (s, 1H), 7.90 (d, J = 2.3 Hz, 1H, 7.50 (m, 1H), 7.35 (m, 4H), 7.20 (m, 5H), 5.31 (m, 1H), 3.55 (m, 1H), 2.75 (ddd, J = 18.7, 5.7, 1.8 Hz, 1H), 2.43 (s, 3H). |
| 4 | 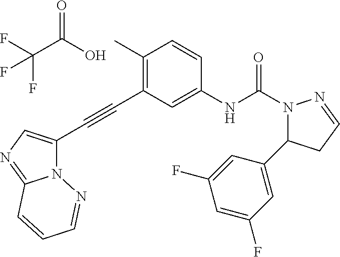 | 5-(3,5-difluorophenyl)- N-(3-(imidazo[1,2- b]pyridazin-3- ylethynyl)-4- methylphenyl)-4,5- dihydro-1H-pyrazole-1- carboxamide 2,2,2- trifluoroacetate | 457.2; 1.76 | ¹H NMR (400 MHz, DMSO-d₆) δ 9.22 (s, 1H), 8.71 (dd, J = 4.5, 1.6 Hz, 1H), 8.24 (dd, J = 9.2, 1.6 Hz, 1H), 8.19 (s, 1H), 7.90 (d, J = 2.3 Hz, 1H), 7.51 (dd, J = 8.4, 2.4 Hz, 1H), 7.38 (dd, J = 9.2, 4.5 Hz, 1H), 7.22 (d, J = 8.4 Hz, 1H), 7.18 (m, 1H), 7.14 (tt, J = 9.3, 2.4 Hz, 1H), 6.94 (m, 2H), 5.35 (dd, J = 12.1, 6.2 Hz, 1H), 3.54 (ddd, J = 18.8, 12.1, 1.7 Hz, 1H), 2.81 (ddd, J = 18.8, 6.2, 1.8 Hz, 1H), 2.44 (s, 3H). |
| 5 | 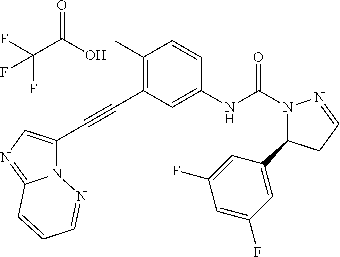 | (S)-5-(3,5- difluorophenyl)-N-(3- (imidazo[1,2- b]pyridazin-3- ylethynyl)-4- methylphenyl)-4,5- dihydro-1H-pyrazole-1- carboxamide 2,2,2- trifluoroacetate | 457.2; 1.76 | ¹H NMR (400 MHz, DMSO-d₆) δ 9.22 (s, 1H), 8.71 (dd, J = 4.5, 1.6 Hz, 1H), 8.24 (dd, J = 9.2, 1.6 Hz, 1H), 8.19 (s, 1H), 7.90 (d, J = 2.3 Hz, 1H), 7.51 (dd, J = 8.4, 2.4 Hz, 1H), 7.38 (dd, J = 9.2, 4.5 Hz, 1H), 7.22 (d, J = 8.4 Hz, 1H), 7.18 (m, 1H), 7.14 (tt, J = 9.3, 2.4 Hz, 1H), 6.94 (m, 2H), 5.35 (dd, J = 12.1, 6.2 Hz, 1H), 3.54 (ddd, J = 18.8, 12.1, 1.7 Hz, 1H), 2.81 (ddd, J = 18.8, 6.2, 1.8 Hz, 1H), 2.44 (s, 3H). |
| 6 | 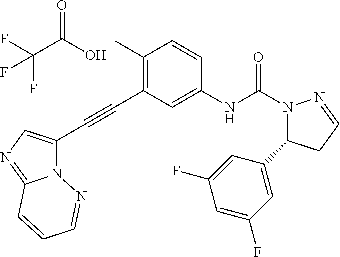 | (R)-5-(3,5- difluorophenyl)-N-(3- (imidazo[1,2- b]pyridazin-3- ylethynyl)-4- methylphenyl)-4,5- dihydro-1H-pyrazole-1- carboxamide 2,2,2- trifluoroacetate | 457.2; 1.76 | ¹H NMR (400 MHz, DMSO-d₆) δ 9.22 (s, 1H), 8.71 (dd, J = 4.5, 1.6 Hz, 1H), 8.24 (dd, J = 9.2, 1.6 Hz, 1H), 8.19 (s, 1H), 7.90 (d, J = 2.3 Hz, 1H), 7.51 (dd, J = 8.4, 2.4 Hz, 1H), 7.38 (dd, J = 9.2, 4.5 Hz, 1H), 7.22 (d, J = 8.4 Hz, 1H), 7.18 (m, 1H), 7.14 (tt, J = 9.3, 2.4 Hz, 1H), 6.94 (m, 2H), 5.35 (dd, J = 12.1, 6.2 Hz, 1H), 3.54 (ddd, J = 18.8, 12.1, 1.7 Hz, 1H), 2.81 (ddd, J = 18.8, 6.2, 1.8 Hz, 1H), 2.44 (s, 3H). |
| 7 | 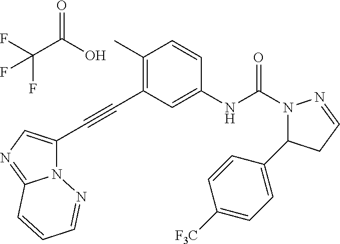 | N-(3-(imidazo[1,2- b]pyridazin-3- ylethynyl)-4- methylphenyl)-5-(4- (trifluoromethyl)phenyl)- 4,5-dihydro-1H- pyrazole-1-carboxamide 2,2,2-trifluoroacetate | 489.2; 1.84 | ¹H NMR (400 MHz, DMSO-d₆) δ 9.24 (s, 1H), 8.70 (dd, J = 4.5, 1.6 Hz, 1H), 8.24 (dd, J = 9.2, 1.6 Hz, 1H), 8.19 (s, 1H), 7.90 (d, J = 2.3 Hz, 1H), 7.73 (d, J = 8.1 Hz, 2H), 7.51 (dd, J = 8.3, 2.4 Hz, 1H), 7.45 (d, J = 8.0 Hz, 2H), 7.38 (dd, J = 9.2, 4.5 Hz, 1H), 5.42 (dd, J = 12.1, 6.2 Hz, 1H), 3.59 (ddd, J = 18.8, 12.2, 1.7 Hz, 1H), 2.79 (ddd, J = 18.8, 6.2, 1.8 Hz, 1H), 2.43 (s, 3H). |
| 8 | 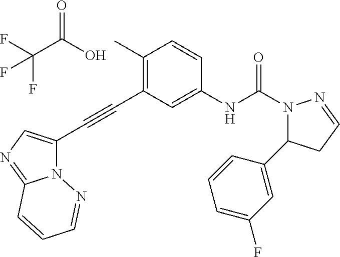 | 5-(3-fluorophenyl)-N-(3- (imidazo[1,2- b]pyridazin-3- ylethynyl)-4- methylphenyl)-4,5- dihydro-1H-pyrazole-1- carboxamide 2,2,2- trifluoroacetate- | 439.3; 1.73 | ¹H NMR (400 MHz, DMSO-d₆) δ 9.16 (s, 1H), 8.71 (dd, J = 4.5, 1.5 Hz, 1H), 8.24 (m, 2H), 7.88 (d, J = 2.3 Hz, 1H), 7.48 (dd, J = 8.4, 2.4 Hz, 1H), 7.39 (m, 1H), 7.35 (m, 1H), 7.17 (d, J = 6.5 Hz, 1H), 7.13 (m, 1H), 7.01 (m, 4H), 5.30 (dd, J = 12.1, 5.9 Hz, 1H), 3.51 (ddd, J = 18.8, 12.1, 1.7 Hz, 1H), 2.74 (ddd, J = 18.7, 6.0, 1.8 Hz, 1H), 2.40 (s, 3H). |
| 9 | 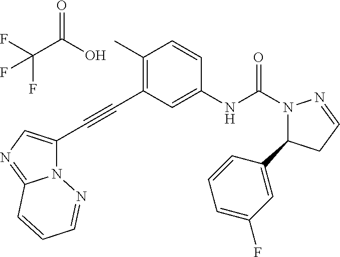 | (S)-5-(3-fluorophenyl)- N-(3-(imidazo[1,2- b]pyridazin-3- ylethynyl)-4- methylphenyl)-4,5- dihydro-1H-pyrazole-1- carboxamide 2,2,2- trifluoroacetate | 439.3; 1.73 | ¹H NMR (400 MHz, DMSO-d₆) δ 9.16 (s, 1H), 8.71 (dd, J = 4.5, 1.5 Hz, 1H), 8.24 (m, 2H), 7.88 (d, J = 2.3 Hz, 1H), 7.48 (dd, J = 8.4, 2.4 Hz, 1H), 7.39 (m, 1H), 7.35 (m, 1H), 7.17 (d, J = 8.5 Hz, 1H), 7.13 (m, 1H), 7.01 (m, 4H), 5.30 (dd, J = 12.1, 5.9 Hz, 1H), 3.51 (ddd, J = 18.8, 12.1, 1.7 Hz, 1H), 2.74 (ddd, J = 18.7, 6.0, 1.8 Hz, 1H), 2.40 (s, 3H). |
| 10 | 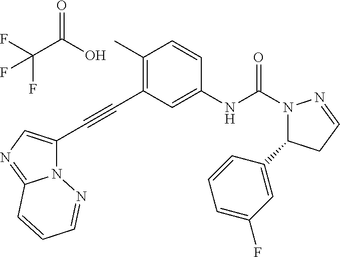 | (R)-5-(3-fluorophenyl)- N-(3-(imidazo[1,2- b]pyridazin-3- ylethynyl)-4- methylphenyl)-4,5- dihydro-1H-pyrazole-1- carboxamide 2,2,2- trifluoroacetate | 439.3; 1.73 | ¹H NMR (400 MHz, DMSO-d₆) δ 9.16 (s, 1H), 8.71 (dd, J = 4.5, 1.5 Hz, 1H), 8.24 (m, 2H), 7.88 (d, J = 2.3 Hz, 1H), 7.48 (dd, J = 8.4, 2.4 Hz, 1H), 7.39 (m, 1H), 7.35 (m, 1H), 7.17 (d, J = 8.5 Hz, 1H), 7.13 (m, 1H), 7.01 (m, 4H), 5.30 (dd, J = 12.1, 5.9 Hz, 1H), 3.51 (ddd, J = 18.8, 12.1, 1.7 Hz, 1H), 2.74 (ddd, J = 18.7, 6.0, 1.8 Hz, 1H), 2.40 (s, 3H). |
| 11 | 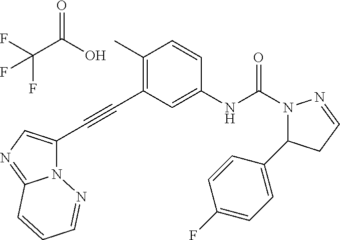 | 5-(4-fluorophenyl)-N-(3- (imidazo[1,2- b]pyridazin-3- ylethynyl)-4- methylphenyl)-4,5- dihydro-1H-pyrazole-1- carboxamide 2,2,2- trifluoroacetate | 439.2; 1.73 | ¹H NMR (400 MHz, DMSO-d₆) δ 9.17 (s, 1H), 8.71 (dd, J = 4.4, 1.6 Hz, 1H), 8.25 (dd, J = 9.2, 1.6 Hz, 1H), 8.21 (s, 1H), 7.90 (d, J = 2.3 Hz, 1H), 7.51 (dd, J = 8.4, 2.4 Hz, 1H), 7.39 (dd, J = 9.2, 4.4 Hz, 1H), 7.21 (m, 7H), 5.33 (dd, J = 12.0, 5.8 Hz, 1H), 3.54 (ddd, J = 18.8, 12.1, 1.7 Hz, 1H), 2.76 (ddd, J = 18.8, 5.9, 1.8 Hz, 1H), 2.43 (s, 3H). |
| 12 | 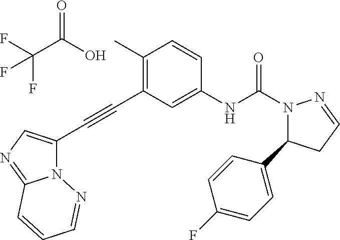 | (S)-5-(4-fluorophenyl)- (imidazo[1,2- b]pyridazin-3- ylethynyl)-4- methylphenyl)-4,5- dihydro-1H-pyrazole-1- carboxamide 2,2,2- trifluoroacetate | 439.2; 1.73 | ¹H NMR (400 MHz, DMSO-d₆) δ 9.17 (s, 1H), 8.71 (dd, J = 4.4, 1.6 Hz, 1H), 8.25 (dd, J = 9.2, 1.6 Hz, 1H), 8.21 (s, 1H), 7.90 (d, J = 2.3 Hz, 1H), 7.51 (dd, J = 8.4, 2.4 Hz, 1H), 7.39 (dd, J = 9.2, 4.4 Hz, 1H), 7.21 (m, 7H), 5.33 (dd, J = 12.0, 5.8 Hz, 1H), 3.54 (ddd, J = 18.8, 12.1, 1.7 Hz, 1H), 2.76 (ddd, J = 18.8, 5.9, 1.8 Hz, 1H), 2.43 (s, 3H). |
| 13 | 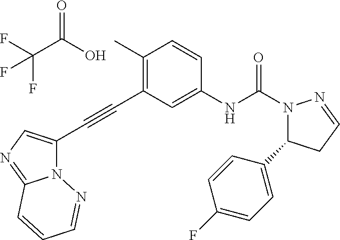 | (R)-5-(4-fluorophenyl)- N-(3-(imidazo[1,2- b]pyridazin-3- ylethynyl)-4- methylphenyl)-4,5- dihydro-1H-pyrazole-1- carboxamide 2,2,2- trifluoroacetate | 439.2; 1.73 | ¹H NMR (400 MHz, DMSO-d₆) δ 9.17 (s, 1H), 8.71 (dd, J = 4.4, 1.6 Hz, 1H), 8.25 (dd, J = 9.2, 1.6 Hz, 1H), 8.21 (s, 1H), 7.90 (d, J = 2.3 Hz, 1H), 7.51 (dd, J = 8.4, 2.4 Hz, 1H), 7.39 (dd, J = 9.2, 4.4 Hz, 1H), 7.21 (m, 7H), 5.33 (dd, J = 12.0, 5.8 Hz, 1H), 3.54 (ddd, J = 18.8, 12.1, 1.7 Hz, 1H), 2.76 (ddd, J = 18.8, 5.9, 1.8 Hz, 1H), 2.43 (s, 3H). |
| 14 | 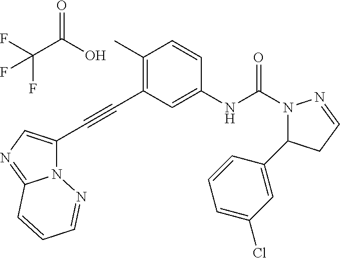 | 5-(3-chlorophenyl)-N-(3- (imidazo[1,2- b]pyridazin-3- ylethynyl)-4- methylphenyl)-4,5- dihydro-1H-pyrazole-1- carboxamide 2,2,2- trifiuoroacetate | 455.2; 1.72 | ¹H NMR (400 MHz, DMSO-d₆) δ 9.21 (s, 1H), 8.72 (dd, J = 1.2, 4.4 Hz, 1H), 8.29-8.18 (m, 2H), 7.90 (d, J = 2.4 Hz, 1H), 7.51 (dd, J = 2.4, 8.4 Hz, 1H), 7.42-7.36 (m, 2H), 7.35-7.31 (m, 1H), 7.26 (s, 1H), 7.22-7.17 (m, 3H), 5.32 (br d, J = 6.4 Hz, 1H), 3.54 (m, 1H), 2.78 (m, 1H), 2.46-2.40 (m, 3H). |
| 15 | 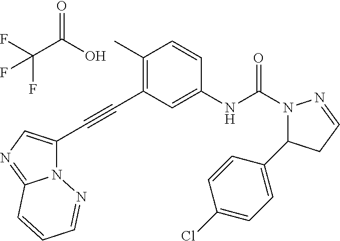 | 5-(4-chlorophenyl)-N-(3- (imidazo[1,2- b]pyridazin-3- ylethynyl)-4- methyiphenyl)-4,5- dihydro-1H-pyrazole-1- carboxamide 2,2,2- trifluoroacetate | 455.2; 1.73 | ¹H NMR (400 MHz, DMSO-d₆) δ 9.21 (s, 1H), 8.72 (dd, J = 1.2, 4.4 Hz, 1H), 8.29-8.18 (m, 2H), 7.90 (d, J = 2.4 Hz, 1H), 7.51 (dd, J = 2.4, 8.4 Hz, 1H), 7.42-7.36 (m, 2H), 7.35-7.31 (m, 1H), 7.26 (s, 1H), 7.22-7.17 (m, 3H), 5.32 (br d, J = 6.4 Hz, 1H), 3.54 (m, 1H), 2.78 (m, 1H), 2.46-2.40 (m, 3H). |
| 16 | 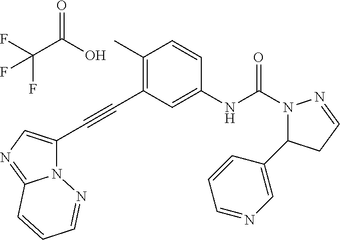 | N-(3-(imidazo[1,2- b]pyridazin-3- ylethynyl)-4- methylphenyl)-5- (pyridin-3-yl)-4,5- dihydro-1H-pyrazole-1- carboxamide 2,2,2- trifluoroacetate | 422.3; 1.12 | ¹H NMR (400 MHz, DMSO-d₆) δ 9.34-9.28 (m, 1H), 8.92-8.66 (m, 3H), 8.28-8.16 (m, 3H), 7.90-7.81 (m, 2H), 7.50 (dd, J = 2.3, 8.4 Hz, 1H), 7.38 (dd, J = 4.4, 9.2 Hz, 1H), 7.27-7.24 (m, 1H), 7.23-7.18 (m, 1H), 5.54-5.44 (m, 1H), 3.60 (m, 2H), 2.99-2.89 (m, 1H), 2.44-2.40 (m, 3H). |
| 17 | 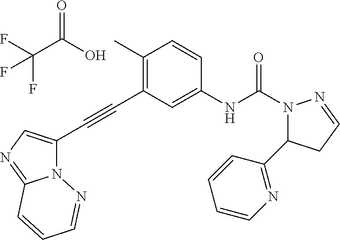 | N-(3-(imidazo[1,2- b]pyridazin-3- ylethynyl)-4- methylphenyl)-5- (pyridin-2-dihydro-1H- pyrazole-1-carboxamide 2,2,2-trifluoroacetate | 422.4; 1.27 | ¹H NMR (400 MHz, DMSO-d₆) δ 9.24 (s, 1H), 8.73 (d, J = 4.4 Hz, 1H), 8.64 (br d, J = 4.4 Hz, 1H), 8.34-8.22 (m, 1H), 8.04 (m, 1H), 7.89 (d, J = 2.4 Hz, 1H), 7.57-7.49 (m, 3H), 7.42 (br s, 1H), 7.24-7.18 (m, 2H), 5.46 (m, 1H, 3.54 (m, 1H), 3.02 (m, 1H), 2.43 (s, 3H). |
| 18 | 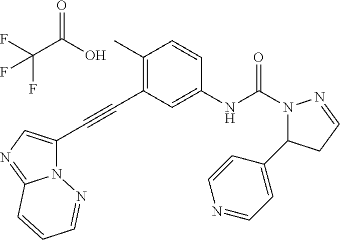 | N-(3-(imidazo[1,2- b]pyridazin-3- ylethynyl)-4- methylphenyl)-5- (pyridin-4-dihydro-1H- pyrazole-1-carboxamide 2,2,2-trifluoroacetate | 422.3; 1.0919 | ¹H NMR (400 MHz, DMSO-d₆) δ 9.42-9.33 (m, 1H), 8.90-8.79 (m, 2H), 8.74-8.67 (m, 1H), 8.27-8.22 (m, 1H, 8.19 (s, 1H), 7.89 (d, J = 2.4 Hz, 1H), 7.85-7.79 (m, 2H), 7.54-7.49 (m, 1H), 7.38 (dd, J = 4.6, 9.2 Hz, 1H), 7.27-7.24 (m, 1H), 7.24-7.19 (m, 1H), 5.57-5.49 (m, 1H), 3.70-3.56 (m, 1H), 2.93-2.81 (m, 1H), 2.48-2.40 (m, 3H). |
| 19 | 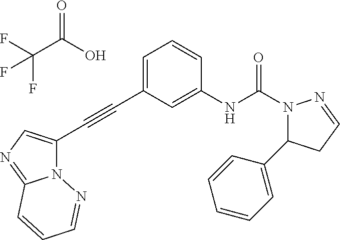 | N-(3-(imidazo[1,2- b]pyridazin-3- ylethynyl)phenyl)-5- phenyl-4,5-dihydro-1H- pyrazole-1-carboxamide 2,2,2-trifluoroacetate | 407.2; 1.65 | ¹H NMR (400 MHz, DMSO-d₆) δ 9.29 (s, 1H), 8.70 (dd, J = 4.5, 1.5 Hz, 1H), 8.24 (dd, J = 9.2, 1.6 Hz, 1H), 8.19 (s, 1H), 7.96 (t, J = 1.9 Hz, 1H), 7.62 (ddd, J = 8.3, 2.3, 1.1 Hz, 1H), 7.27 (m, 10H), 5.33 (dd, J = 12.0, 5.6 Hz, 1H), 3.56 (ddd, J = 18.7, 12.0, 1.7 Hz, 1H), 2.76 (ddd, J = 18.8, 5.7, 1.8 Hz, 1H). |
| 20 | 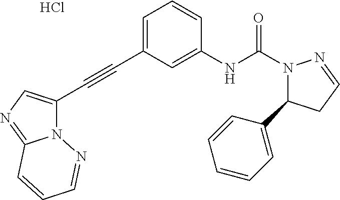 [SMILES: O=C(Nc1cccc(C#Cc2cnc3cccnn23)c1)N1N=CC[C@H]1c1ccccc1 ; Cl] | (S)-N-(3-(imidazo[1,2- b]pyridazin-3- ylethynyl)phenyl)-5- phenyl-4,5-dihydro-1H- pyrazole-1-carboxamide hydrochoride | 407.2; 1.65 | ¹H NMR (400 MHz, DMSO-d₆) δ 9.29 (s, 1H), 8.70 (dd, J = 4.5, 1.5 Hz, 1H), 8.24 (dd, J = 9.2, 1.6 Hz, 1H), 8.19 (s, 1H), 7.96 (t, J = 1.9 Hz, 1H), 7.62 (ddd, J = 8.3, 2.3, 1.1 Hz, 1H), 7.27 (m, 10H), 5.33 (dd, J = 12.0, 5.6 Hz, 1H), 3.56 (ddd, J = 18.7, 12.0, 1.7 Hz, 1H), 2.76 (ddd, J = 18.8, 5.7, 1.8 Hz, 1H). |
| 21 | 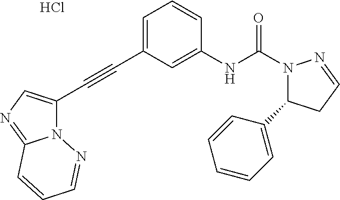 [SMILES: O=C(Nc1cccc(C#Cc2cnc3cccnn23)c1)N1N=CC[C@@H]1c1ccccc1 ; Cl] | (R)-N-(3-(imidazo[1,2- b]pyridazin-3- ylethynyl)phenyl)-5- phenyl-4,5-dihydro-1H- pyrazole-1-carboxamide hydrochoride | 407.2; 1.65 | ¹H NMR (400 MHz, DMSO-d₆) δ 9.29 (s, 1H), 8.70 (dd, J = 4.5, 1.5 Hz, 1H), 8.24 (dd, J = 9.2, 1.6 Hz, 1H), 8.19 (s, 1H), 7.96 (t, J = 1.9 Hz, 1H), 7.62 (ddd, J = 8.3, 2.3, 1.1 Hz, 1H), 7.27 (m, 10H), 5.33 (dd, J = 12.0, 5.6 Hz, 1H), 3.56 (ddd, J = 18.7, 12.0, 1.7 Hz, 1H), 2.76 (ddd, J = 18.8, 5.7, 1.8 Hz, 1H). |
| 22 | 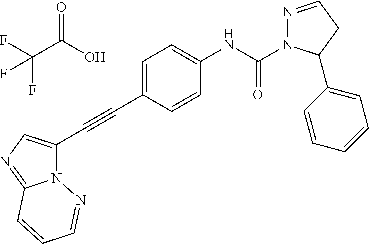 | N-(4-(imidazo[1,2- b]pyridazin-3- ylethynyl)phenyl)-5- phenyl-4,5-dihydro-1H- pyrazole-1-carboxamide 2,2,2-trifluoroacetate | 407.2; 1.59 | ¹H NMR (400 MHz, DMSO-d₆) δ 9.37 (s, 1H), 8.68 (dd, J = 4.5, 1.6 Hz, 1H), 8.22 (dd, J = 9.2, 1.6 Hz, 1H), 8.14 (s, 1H), 7.72 (m, 2H), 7.46 (m, 2H), 7.35 (m, 3H), 7.23 (m, 4H), 5.34 (dd, J = 12.0, 5.6 Hz, 1H), 3.56 (ddd, J = 18.8, 12.1, 1.7 Hz, 1H), 2.76 (ddd, J = 18.8, 5.6, 1.8 Hz, 1H). |
| 23 | 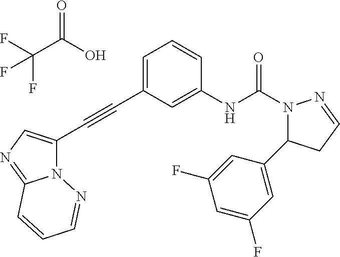 | 5-(3,5-difluorophenyl)- N-(3-(imidazo[1,2- b]pyridazin-3- ylethynyl)phenyl)-4,5- dihydro-1H-pyrazole-1- carboxamide 2,2,2- trifluoroacetate | 443.2; 1.68 | ¹H NMR (400 MHz, DMSO-d₆) δ 9.34 (s, 1H), 8.72 (dd, J = 4.4, 1.6 Hz, 1H), 8.26 (dd, J = 9.2, 1.6 Hz, 1H), 8.22 (s, 1H), 7.98 (t, J = 1.9 Hz, 1H), 7.64 (ddd, J = 8.3, 2.3, 1.1 Hz, 1H), 7.41 (dd, J = 9.2, 4.5 Hz, 1H), 7.33 (t, J = 7.9 Hz, 1H), 7.21 (td, J = 3.1, 1.4 Hz, 2H), 7.14 (tt, J = 9.3, 2.4 Hz, 2H), 6.95 (m, 3H), 5.36 (dd, J = 12.1, 6.1 Hz, 1H), 3.55 (ddd, J = 18.8, 12.1, 1.7 Hz, 1H), 2.82 (ddd, J = 18.8, 6.2, 1.8 Hz, 1H). |
| 24 | 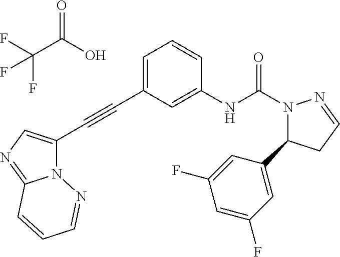 | (S)-5-(3,5- difluorophenyl)-N-(3- (imidazo[1,2- b]pyridazin-3- ylethynyl)phenyl)-4,5- dihydro-1H-Pyrazole-1- carboxamide 2,2,2- trifluoroacetate | 443.2; 1.68 | ¹H NMR (400 MHz, DMSO-d₆) δ 9.34 (s, 1H), 8.72 (dd, J = 4.4, 1.6 Hz, 1H), 8.26 (dd, J = 9.2, 1.6 Hz, 1H), 8.22 (s, 1H), 7.98 (t, J = 1.9 Hz, 1H), 7.64 (ddd, J = 8.3, 2.3, 1.1 Hz, 1H), 7.41 (dd, J = 9.2, 4.5 Hz, 1H), 7.33 (t, J = 7.9 Hz, 1H), 7.21 (td, J = 3.1, 1.4 Hz, 2H), 7.14 (tt, J = 9.3, 2.4 Hz, 2H), 6.95 (m, 3H), 5.36 (dd, J = 12.1, 6.1 Hz, 1H), 3.55 (ddd, J = 18.8, 12.1, 1.7 Hz, 1H), 2.82 (ddd, J = 18.8, 6.2, 1.8 Hz, 1H). |
| 25 | 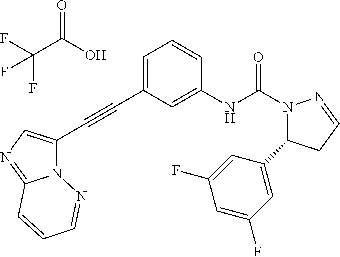 | (R)-5-(3,5- difluorophenyl)-N-(3- (imidazo[1,2- b]pyridazin-3- ylethynyl)phenyl)-4,5- dihydro-1H-pyrazole-1- carboxamide 2,2,2- trifluoroacetate | 443.2; 1.68 | ¹H NMR (400 MHz, DMSO-d₆) δ 9.34 (s, 1H), 8.72 (dd, J = 4.4, 1.6 Hz, 1H), 8.26 (dd, J = 9.2, 1.6 Hz, 1H), 8.22 (s, 1H), 7.98 (t, J = 1.9 Hz, 1H), 7.64 (ddd, J = 8.3, 2.3, 1.1 Hz, 1H), 7.41 (dd, J = 9.2, 4.5 Hz, 1H), 7.33 (t, J = 7.9 Hz, 1H), 7.21 (td, J = 3.1, 1.4 Hz, 2H), 7.14 (tt, J = 9.3, 2.4 Hz, 2H), 6.95 (m, 3H), 5.36 (dd, J = 12.1, 6.1 Hz, 1H), 3.55 (ddd, J = 18.8, 12.1, 1.7 Hz, 1H), 2.82 (ddd, J = 18.8, 6.2, 1.8 Hz, 1H). |
| 26 | 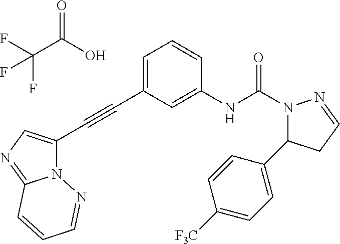 | N-(3-(imidazo[1,2- b]pyridazin-3- ylethynyl)phenyl)-5-(4- (trifiuoromethyl)phenyl)- 4,5-dihydro-1H- pyrazole-1-carboxamide 2,2,2-trifluoroacetate | 475.2; 1.75 | ¹H NMR (400 MHz, DMSO-d₆) δ 9.31 (s, 1H), 8.65 (dd, J = 4.5, 1.5 Hz, 1H), 8.19 (dd, J = 9.2, 1.6 Hz, 1H), 8.15 (s, 1H), 7.92 (t, J = 1.9 Hz, 1H), 7.69 (d, J = 8.1 Hz, 2H), 7.58 (ddd, J = 8.3, 2.3, 1.1 Hz, 1H), 7.41 (d, J = 8.1 Hz, 2H), 7.34 (dd, J = 9.2, 4.5 Hz, 1H), 7.27 (t, J = 7.9 Hz, 1H), 7.16 (m, 2H), 5.39 (dd, J = 12.1, 6.1 Hz, 1H), 3.55 (ddd, J = 18.8, 12.2, 1.7 Hz, 1H), 2.75 (ddd, J = 18.8, 6.1, 1.8 Hz, 1H). |
| 27 | 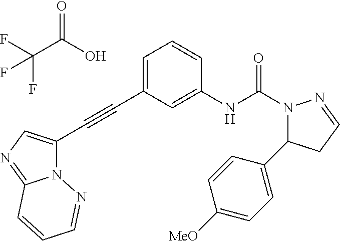 | N-(3-(imidazo[1,2- b]pyridazin-3- ylethynyl)phenyl)-5-(4- methoxyphenyl)-4,5- dihydro-1H-pyrazole-1- carboxamide 2,2,2- trifluoroacetate | 437.2; 1.61 | ¹H NMR (400 MHz, DMSO-d₆) δ 9.24 (s, 1H), 8.71 (dd, J = 4.4, 1.5 Hz, 1H), 8.24 (dd, J = 9.2, 1.6 Hz, 1H), 8.20 (s, 1H), 7.97 (t, J = 1.9 Hz, 1H), 7.62 (ddd, J = 8.3, 2.2, 1.1 Hz, 1H), 7.39 (dd, J = 9.2, 4.5 Hz, 1H), 7.31 (t, J = 7.9 Hz, 1H), 7.17 (m, 4H), 6.90 (m, 2H), 5.27 (m, 1H), 3.73 (s, 3H), 3.52 (m, 1H), 2.75 (ddd, J = 18.7, 5.6, 1.8 Hz, 1H). |
| 28 | 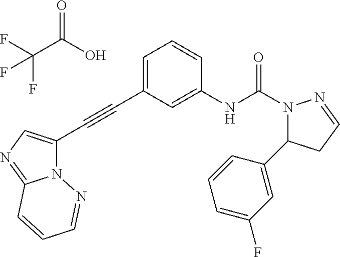 | 5-(3-fluorophenyl)-N-(3- (imidazo[1,2- b]pyridazin-3- ylethynyl)phenyl)-4,5- dihydro-1H-pyrazole-1- carboxamide 2,2,2- trifluoroacetate | 425.3; 1.66 | ¹H NMR (400 MHz, DMSO-d₆) δ 9.32 (s, 1H), 8.74 (dd, J = 4.4, 1.6 Hz, 1H), 8.27 (dd, J = 9.2, 1.6 Hz, 1H), 8.25 (s, 1H), 7.97 (t, J = 1.9 Hz, 1H), 7.63 (ddd, J = 8.3, 2.2, 1.1 Hz, 1H), 7.39 (m, 3H), 7.19 (dt, J = 6.6, 1.5 Hz, 2H), 7.07 (m, 3H), 5.35 (dd, J = 12.1, 5.9 Hz, 1H), 3.56 (ddd, J = 18.8, 12.2, 1.7 Hz, 1H), 2.79 (ddd, J = 18.7, 5.9, 1.8 Hz, 1H). |
| 29 | 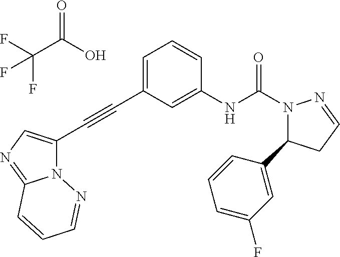 | (S)-5-(3-fluorophenyl)- N-(3-(imidazo[1,2- b]pyridazin-3- ylethynyl)phenyl)-4,5- dihydro-1H-pyrazole-1- carboxamide 2,2,2- trifluoroacetate | 425.3; 1.66 | ¹H NMR (400 MHz, DMSO-d₆) δ 9.32 (s, 1H), 8.74 (dd, J = 4.4, 1.6 Hz, 1H), 8.27 (dd, J = 9.2, 1.6 Hz, 1H), 8.25 (s, 1H), 7.97 (t, J = 1.9 Hz, 1H), 7.63 (ddd, J = 8.3, 2.2, 1.1 Hz, 1H), 7.39 (m, 3H), 7.19 (dt, J = 6.6, 1.5 Hz, 2H), 7.07 (m, 3H), 5.35 (dd, J = 12.1, 5.9 Hz, 1H), 3.56 (ddd, J = 18.8, 12.2, 1.7 Hz, 1H), 2.79 (ddd, J = 18.7, 5.9, 1.8 Hz, 1H). |
| 30 | 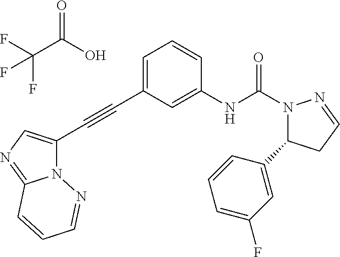 | (R)-5-(3-fluorophenyl)- N-(3-(imidazo[1,2- b]pyridazin-3- ylethynyl)phenyl)-4,5- dihydro-1H-pyrazole-1- carboxamide 2,2,2- trifluoroacetate | 425.3; 1.66 | ¹H NMR (400 MHz, DMSO-d₆) δ 9.32 (s, 1H), 8.74 (dd, J = 4.4, 1.6 Hz, 1H), 8.27 (dd, J = 9.2, 1.6 Hz, 1H), 8.25 (s, 1H), 7.97 (t, J = 1.9 Hz, 1H), 7.63 (ddd, J = 8.3, 2.2, 1.1 Hz, 1H), 7.39 (m, 3H), 7.19 (dt, J = 6.6, 1.5 Hz, 2H), 7.07 (m, 3H), 5.35 (dd, J = 12.1, 5.9 Hz, 1H), 3.56 (ddd, J = 18.8, 12.2, 1.7 Hz, 1H), 2.79 (ddd, J = 18.7, 5.9, 1.8 Hz, 1H). |
| 31 | 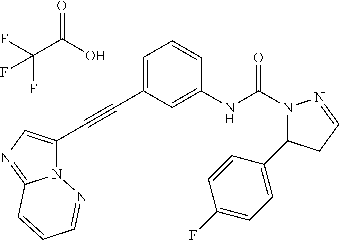 | 5-(4-fluorophenyl)-N-(3- (imidazo[1,2- b]pyridazin-3- ylethynyl)phenyl)-4,5- dihydro-1H-pyrazole-1- carboxamide 2,2,2- trifluoroacetate | 425.2; 1.66 | ¹H NMR (400 MHz, DMSO-d₆) δ 9.30 (s, 1H), 8.74 (dd, J = 4.5, 1.5 Hz, 1H), 8.26 (m, 2H), 7.98 (t, J = 1.9 Hz, 1H), 7.64 (ddd, J = 8.3, 2.3, 1.1 Hz, 1H), 7.42 (dd, J = 9.2, 4.4 Hz, 1H), 7.29 (m, 4H), 7.19 (m, 5H), 5.35 (dd, J = 12.0, 5.8 Hz, 1H), 3.56 (ddd, J = 18.8, 12.0, 1.7 Hz, 1H), 2.77 (ddd, J = 18.8, 5.8, 1.8 Hz, 1H). |
| 32 | 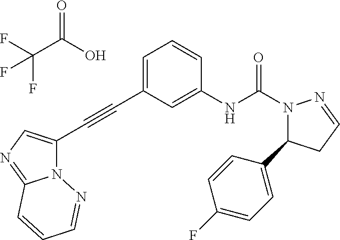 | (S)-5-(4-fluorophenyl)- N-(3-(imidazo[1,2- b]pyridazin-3- ylethynyl)phenyl)-4,5- dihydro-1H-pyrazole-1- carboxamide 2,2,2- trifluoroacetate | 425.2; 1.66 | ¹H NMR (400 MHz, DMSO-d₆) δ 9.30 (s, 1H), 8.74 (dd, J = 4.5, 1.5 Hz, 1H), 8.26 (m, 21-I), 7.98 (t, J = 1.9 Hz, 1H), 7.64 (ddd, J = 8.3, 2.3, 1.1 Hz, 1H), 7.42 (dd, J = 9.2, 4.4 Hz, 1H), 7.29 (m, 4H), 7.19 (m, 5H), 5.35 (dd, J = 12.0, 5.8 Hz, 1H), 3.56 (ddd, J = 18.8, 12.0, 1.7 Hz, 1H), 2.77 (ddd, J = 18.8, 5.8, 1.8 Hz, 1H). |
| 33 | 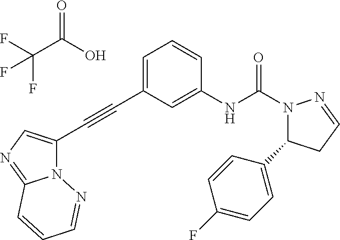 | (R)-5-(4-fluorophenyl)- N-(3-(imidazo[1,2- b]pyridazin-3- ylethynyl)phenyl)-4,5- dihydro-1H-pyrazole-1- carboxamide 2,2,2- trifluoroacetate | 425.2; 1.66 | ¹H NMR (400 MHz, DMSO-d₆) δ 9.30 (s, 1H), 8.74 (dd, J = 4.5, 1.5 Hz, 1H), 8.26 (m, 2H), 7.98 (t, J = 1.9 Hz, 1H), 7.64 (ddd, J = 8.3, 2.3, 1.1 Hz, 1H), 7.42 (dd, J = 9.2, 4.4 Hz, 1H), 7.29 (m, 4H), 7.19 (m, 5H), 5.35 (dd, J = 12.0, 5.8 Hz, 1H), 3.56 (ddd, J = 18.8, 12.0, 1.7 Hz, 1H), 2.77 (ddd, J = 18.8, 5.8, 1.8 Hz, 1H). |
| 34 | 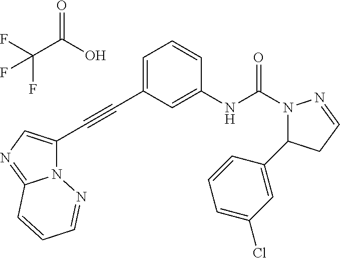 | 5-(3-chlorophenyl)-N-(3- (imidazo[1,2- b]pyridazin-3- ylethynyl)phenyl)-4,5- dihydro-1H-pyrazole-1- carboxamide 2,2,2- trifluoroacetate | 441.2; 1.72 | ¹H NMR (400 MHz, DMSO-d₆) δ 9.33 (s, 1H), 8.72 (dd, J = 4.4, 1.4 Hz, 1H), 8.25 (dd, J = 9.2, 1.6 Hz, 1H), 8.22 (s, 1H), 7.97 (t, J = 1.9 Hz, 1H), 7.64 (ddd, J = 8.4, 2.3, 1.1 Hz, 1H), 7.30 (m, 10H), 5.34 (m, 1H), 3.56 (ddd, J = 18.8, 12.1, 1.7 Hz, 1H), 2.80 (ddd, J = 18.8, 5.9, 1.8 Hz, 1H). |
| 35 | 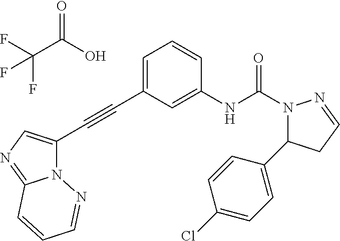 | 5-(4-chlorophenyl)-N-(3- (imidazo[1,2- b]pyridazin-3- ylethynyl)phenyl)-4,5- dihydro-1H-pyrazole-1- carboxamide 2,2,2- trifluoroacetate | 441.2; 1.73 | ¹H NMR (400 MHz, DMSO-d₆) δ 9.31 (s, 1H), 8.71 (dd, J = 4.5, 1.6 Hz, 1H), 8.24 (dd, J = 9.2, 1.6 Hz, 1H), 8.20 (s, 1H), 7.96 (t, J = 1.9 Hz, 1H), 7.62 (ddd, J = 8.3, 2.3, 1.1 Hz, 1H), 7.41 (d, J = 8.4 Hz, 2H), 7.38 (m, 1H), 7.32 (t, J = 7.9 Hz, 1H), 7.25 (m, 2H), 7.19 (m, 2H), 5.33 (m, 1H), 3.55 (ddd, J = 18.8, 12.1, 1.8 Hz, 1H), 2.76 (ddd, J = 18.8, 5.9, 1.8 Hz, 1H). |
| 36 | 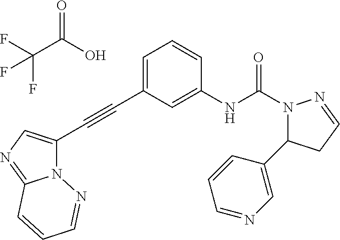 | N-(3-(imidazo[1,2- b]pyridazin-3- ylethynyl)phenyl)-5- (pyridin-3-yl)-4,5- dihydro-1H-pyrazole-1- carboxamide 2,2,2- trifluoroacetate | 408.2; 1.12 | ¹H NMR (400 MHz, DMSO-d₆) δ 9.39 (s, 1H), 8.71 (m, 3H), 8.23 (m, 2H), 8.14 (dd, J = 7.9, 2.0 Hz, 1H), 7.96 (d, J = 1.9 Hz, 1H), 7.81 (s, 1H), 7.63 (ddd, J = 8.3, 2.3, 1.1 Hz, 1H), 7.32 (m, 4H), 5.52 (m, 1H), 3.61 (ddd, 1H), 2.96 (ddd, J = 18.9, 6.7, 1.7 Hz, 1H). |
| 37 | 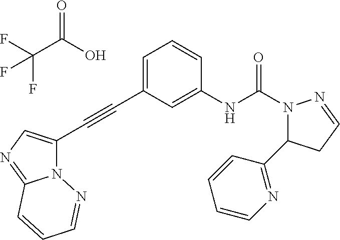 | N-(3-(imidazo[1,2- b]pyridazin-3- ylethynyl)phenyl)-5- (pyridin-2-yl)-4,5- dihydro-1H-pyrazole-1- carboxamide 2,2,2- trifluoroacetate | 408.2; 1.27 | |
| 38 | 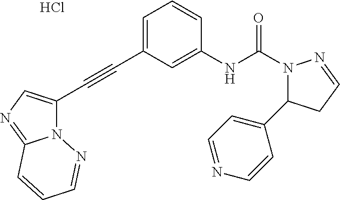 [SMILES: O=C(Nc1cccc(C#Cc2cnc3cccnn23)c1)N1N=CCC1c1ccncc1 ; Cl] | N-(3-(imidazo[1,2- b]pyridazin-3- ylethynyl)phenyl)-5- (pyridin-4-yl)-4,5- dihydro-1H-pyrazole-1- carboxamide hydrochoride | 408.2; 1.09 | |
| 39 | 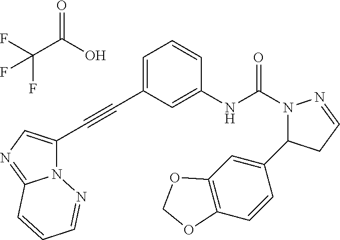 | 5-(benzo[d][1,3]dioxol- 5-yl)-N-(3-(imidazo[1,2- b]pyridazin-3- ylethynyl)phenyl)-4,5- dihydro-1H-pyrazole-1- carboxamide 2,2,2- trifluoroacetate | 451.28; 1.60 | ¹H NMR (400 MHz, DMSO-d₆) δ 9.25 (s, 1H), 8.73 (d, J = 4.3 Hz, 1H), 8.26 (d, J = 8.5 Hz, 2H), 7.98 (s, 1H), 7.63 (dd, J = 8.3, 1.1 Hz, 1H), 7.41 (dd, J = 9.0, 4.4 Hz, 1H), 7.33 (dd, J = 14.1, 6.2 Hz, 1H), 7.19 (d, J = 8.5 Hz, 2H), 6.87 (d, J = 8.0 Hz, 1H), 6.78-6.67 (m, 2H), 5.99 (d, J = 2.3 Hz, 2H), 5.26 (dd, J = 11.9, 5.6 Hz, 1H), 3.51 (ddd, J = 18.6, 12.0, 1.3 Hz, 1H), 2.76 (ddd, J = 18.7, 5.6, 1.5 Hz, 1H). |
| 40 | 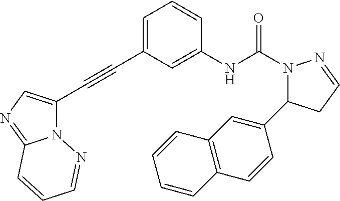 [SMILES: O=C(Nc1cccc(C#Cc2cnc3cccnn23)c1)N1N=CCC1c1ccc2ccccc2c1] | N-(3-(imidazo[1,2- b]pyridazin-3- ylethynyl)phenyl)-5- (naphthalen-2-dihydro- 1H-pyrazole-1- carboxamide | 457.1 | ¹H NMR (400 MHz, DMSO-d₆) δ 9.33 (s, 1H), 8.69 (d, J = 4.4 Hz, 1H), 7.99-7.85 (m, 4H), 7.74 (s, 1H), 7.66-7.59 (m, 1H), 7.56-7.44 (m, 3H), 7.38-7.35 (m, 2H), 7.3 (t, J = 8.0 Hz, H), 7.25 (s, 1H), 7.19-7.15 (m, 1H), 5.50 (dd, J = 6.0, 12.0 Hz, 1H), 3.67-3.59 (m, 1H), 2.90-2.82 (m, 1H). |
| 41 | 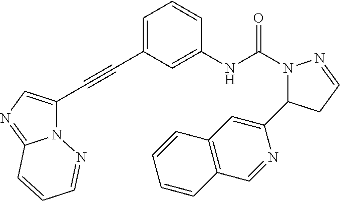 [SMILES: O=C(Nc1cccc(C#Cc2cnc3cccnn23)c1)N1N=CCC1c1cc2ccccc2cn1] | N-(3-(imidazo[1,2- b]pyridazin-3- ylethynyl)phenyl)-5- (isoquinolin-3-yl)-4,5- dihydro-1H-pyrazole-1- carboxamide | 458.4 | ¹H NMR (400 MHz, DMSO-d₆) δ 9.41 (s, 1H), 9.33 (s, 1H), 8.69 (dd, J = 1.2, 4.4 Hz, 1H), 8.26-8.16 (m, 3H), 8.06 (d, J = 8.2 Hz, 1H), 7.95 (t, J = 1.6 Hz, 1H), 7.90-7.83 (m, 2H), 7.75-7.70 (m, 1H), 7.64-7.60 (m, 1H), 7.38 (dd, J = 4.4, 9.2 Hz, 1H), 7.30 (t, J = 8.0 Hz, 1H), 7.26 (s, 1H), 7.17 (d, J = 7.6 Hz, 1H), 5.58 (dd, J = 6.4, 12.0 Hz, 1H), 3.637-3.56 (s, 1H), 3.112-3.045 (m, 1H). |
| 42 | 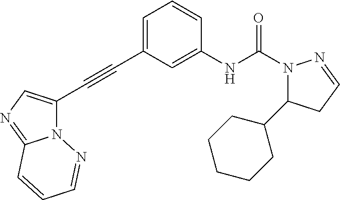 [SMILES: O=C(Nc1cccc(C#Cc2cnc3cccnn23)c1)N1N=CCC1C1CCCCC1] | 5-cyclohexyl-N-(3- (imidazo[1,2- b]pyridazin-3- ylethynyl)phenyl)-4,5- dihydro-1H-pyrazole-1- carboxamide | 413.4 | ¹H NMR (400 MHz, DMSO-d₆) δ 9.12-9.06 (m, 1H), 8.74-8.68 (m, 1H), 8.28-8.18 (m, 2H), 8.05-8.01 (m, 1H), 7.68-7.61 (m, 1H), 7.41-7.36 (m, 1H), 7.33 (t, J = 7.9 Hz, 1H), 7.21-7.17 (m, 1H), 7.08-7.05 (m, 1H), 4.28-4.19 (m, 1H), 3.01-2.91 (m, 1H), 2.82-2.72 (m, 1H), 2.05-1.94 (m, 1H), 1.76-1.66 (m, 2H), 1.57-1.48 (m, 1H), 1.40-1.33 (m, LH), 1.28-1.14 (m, 2H), 1.14-1.02 (m, 2H), 1.01-0.86 (m, 2H). |
| 43 | 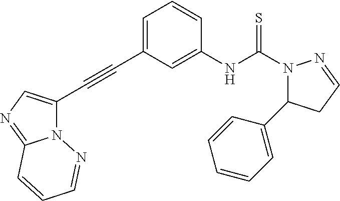 [SMILES: S=C(Nc1cccc(C#Cc2cnc3cccnn23)c1)N1N=CCC1c1ccccc1] | N-(3-(imidazo[1,2- b]pyridazin-3- ylethynyl)phenyl)-5- phenyl-4,5-dihydro-1H- pyrazole-1- carbothioamide | 423.2; 1.62 | |

### EXAMPLE 44

### Preparation of N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate

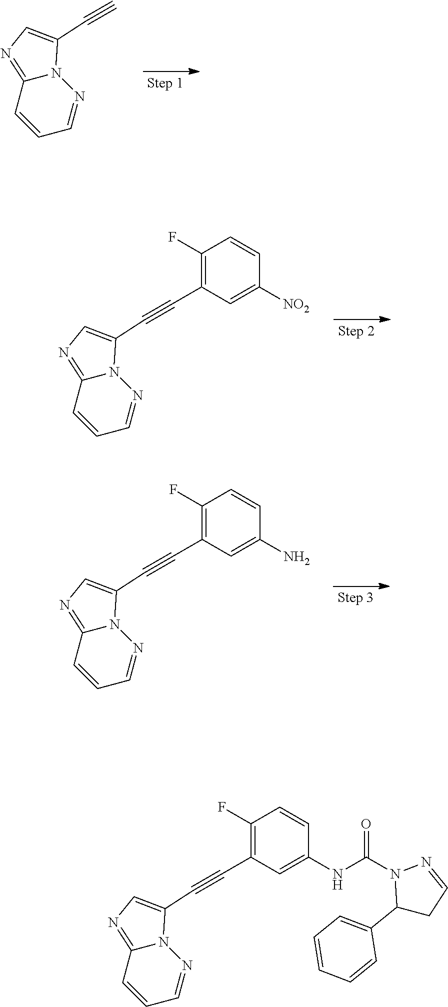 [SMILES: C#Cc1cnc2cccnn12 ; O=[N+]([O-])c1ccc(F)c(C#Cc2cnc3cccnn23)c1 ; Nc1ccc(F)c(C#Cc2cnc3cccnn23)c1 ; O=C(Nc1ccc(F)c(C#Cc2cnc3cccnn23)c1)N1N=CCC1c1ccccc1]

### Step 1: Preparation of 3-((2-fluoro-5-nitrophenyl)ethynyl)imidazo[1,2-b]pyridazine

The compound 3-ethynylimidazo[1,2-b]pyridazine (400 mg, 2.79 mmol) obtained in step 2 of Example 1 and 1-fluoro-2-iodo-4-nitrobenzene (746 mg, 2.79 mmol) were dissolved in ethyl acetate (9.3 ml), followed by ultrasonication for 5 minutes while blowing nitrogen. Pd(PPh₃)₄ (161 mg, 0.140 mmol), CuI (53.2 mg, 0.279 mmol) and DIPEA (976 μl, 5.59 mmol) were added to the reaction mixture, followed by stirring at 50° C. for 16 hours. The reaction mixture was filtered with celite and washed with ethyl acetate. The obtained filtrate was concentrated under reduced pressure, and then purified by medium pressure liquid chromatography (dichloromethane/ethyl acetate) to give the target compound 3-((2-fluoro-5-nitrophenyl)ethynyl)imidazo[1,2-b]pyridazine (619 mg, 78%) as a solid.

MS (m/z): 283.36 [M+1]⁺, UPLC r. t. (min): 2.40

### Step 2: Preparation of 4-fluoro-3-(imidazo[1,2-b]pyridazin-3-ylethynyl)aniline

The compound 3-((2-fluoro-5-nitrophenyl)ethynyl)imidazo[1,2-b]pyridazine obtained in step 1 above (619 mg, 2.193 mmol), Fe (612 mg, 10.97 mmol) and ammonium chloride (1173 mg, 21.93 mmol) were dissolved in a mixed solution of ethanol/H₂O (ratio: 4/1). After heating the mixture at 80° C. for 1 hour and 30 minutes, the temperature was lowered to room temperature to terminate the reaction. The reaction mixture was filtered with celite. The filtrate was concentrated, extracted with chloroform and distilled water, and dried over sodium sulfate. The obtained filtrate was concentrated under reduced pressure using a rotary evaporator to give the target compound 4-fluoro-3-(imidazo[1,2-b]pyridazin-3-ylethynyl)aniline (535 mg, 97%).

MS (m/z): 253.18 [M+1]⁺, UPLC r. t. (min): 1.16

¹H NMR (400 MHz, DMSO-d₆) δ 8.69 (dd, J=4.4, 1.6 Hz, 1H), 8.28-8.14 (m, 2H), 7.37 (dd, J=9.2, 4.5 Hz, 1H), 7.00 (t, J=9.2 Hz, 1H), 6.77 (dd, J=6.0, 2.9 Hz, 1H), 6.64 (ddd, J=8.9, 4.4, 2.9 Hz, 1H), 5.18 (s, 2H).

### Step 3: Preparation of N-(4-fluoro-3-(imidazo[1,2-b]pyridazin-3-ylethynyl)phenyl)-5-phenyl-4,5-dihydro-1H-pyrazole-1-carboxamide trifluoroacetate

The compound 4-fluoro-3-(imidazo[1,2-b]pyridazin-3-ylethynyl)aniline obtained in step 2 above (100 mg, 0.396 mmol), 1,1′-carbonyldiimidazole (96 mg, 0.595 mmol) and DIPEA (277 μl, 1.586 mmol) were dissolved in DMF (1.321 ml), followed by stirring for 1 hour and 30 minutes. 5-Phenyl-4,5-dihydro-1H-pyrazole (103 μl, 0.595 mmol) was added thereto, followed by stirring for 1 hour. Upon completion of the reaction, the reaction mixture was extracted with dichloromethane and distilled water, and the organic layers were combined. The organic layer was dried over sodium sulfate, concentrated under reduced pressure, and purified using a Prep-150 device to give the target compound N-(4-fluoro-3-(imidazo[1,2-b]pyridazin-3-ylethynyl)phenyl)-5-phenyl-4,5-dihydro-1H-pyrazole-1-carboxamide trifluoroacetate (119 mg, 56%).

MS (m/z): 425.29 [M+1]⁺, UPLC r. t. (min): 1.65

### EXAMPLE 45

### Preparation of (S)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate; and

### EXAMPLE 46

### (R)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate

The compound of Example 44 prepared in the above step was separated into (S)—N-(4-fluoro-3-(imidazo[1,2-b]pyridazin-3-ylethynyl)phenyl)-5-phenyl-4,5-dihydro-1H-pyrazole-1-carboxamide trifluoroacetate (Example 45) and (R)—N-(4-fluoro-3-(imidazo[1,2-b]pyridazin-3-ylethynyl)phenyl)-5-phenyl-4,5-dihydro-1H-pyrazole-1-carboxamide trifluoroacetate (Example 46) using supercritical fluid chromatography (chiralcel IB-3 250×20 mm, 40% MeOH containing 0.05% DEA in CO₂, 3 mL/min).

The compound of Example 47 was prepared by the similar manner to the method described in Example 44. The compound names, chemical formulas, and UPLC/NMR analysis results of the compounds of Examples 44 to 47 are summarized in Table 2 below.

TABLE 2

| | | | UPLC | |
|---|---|---|---|---|
| | | | [M + 1]⁺; | |
| | | | r.t. | |
| Example | Structure | Compound Name | (min) | ¹H NMR |
| 44 | 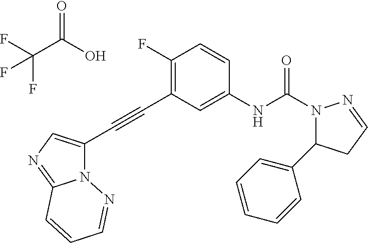 [SMILES: O=C(O)C(F)(F)F ; O=C(Nc1ccc(F)c(C#Cc2cnc3cccnn23)c1)N1N=CCC1c1ccccc1] | N-(4-fluoro-3- (imidazo[1,2- b]pyridazin-3- ylethynyl)phenyl)- 5-phenyl-4,5- dihydro-1H- pyrazole-1- carboxamide 2,2,2- trifluoroacetate | 425.2; 1.66 | ¹H NMR (400 MHz, DMSO-d₆) δ 9.37 (s, 1H), 8.72 (d, J = 3.4 Hz, 1H), 7.99 (dd, J = 6.3, 2.5 Hz, 1H), 7.69- 7.61 (m, 1H), 7.41 (dd, J = 9.2, 4.4 Hz, 1H), 7,35 (t, J = 7.4 Hz, 2H), 7.29- 7.16 (m, 5H), 5.33 (dd, J = 12.0, 5.6 Hz, 1H), 3.56 (dd, J = 18.3, 12.7 Hz, 1H), 2.76 (dd, |
| | | | | J = 18.7, 4.6 Hz, |
| | | | | 1H). |
| 45 | 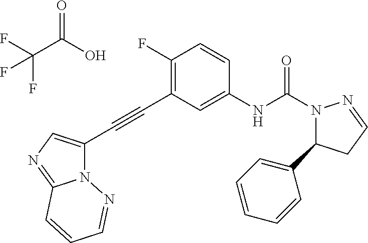 [SMILES: O=C(O)C(F)(F)F ; O=C(Nc1ccc(F)c(C#Cc2cnc3cccnn23)c1)N1N=CC[C@H]1c1ccccc1] | (S)-N-(4-fluoro- 3-(imidazo[1,2- b]pyridazin-3- ylethynyl)phenyl)- 5-phenyl-4,5- dihydro-1H- pyrazole-1- carboxamide 2,2,2- trifluoroacetate | 425.2; 1.66 | ¹H NMR (400 MHz, DMSO-d₆) δ 9.37 (s, 1H), 8.72 (d, J = 3.4 Hz, 1H), 7.99 (dd, J = 6.3, 2.5 Hz, 1H), 7.69- 7.61 (m, 1H), 7.41 (dd, J = 9.2, 4.4 Hz, 1H), 7.35 (t, J = 7.4 Hz, 2H), 7.29- 7.16 (m, 5H), 5.33 (dd, J = 12.0, 5.6 Hz, 1H), 3.56 (dd, J = 18.3, 12.7 Hz, 1H), 2.76 (dd, |
| | | | | J = 18,7, 4.6 Hz, |
| | | | | 1H). |
| 46 | 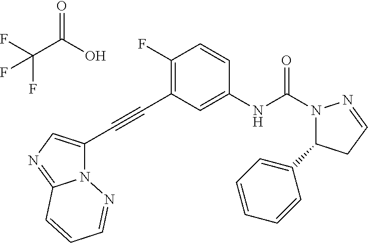 [SMILES: O=C(O)C(F)(F)F ; O=C(Nc1ccc(F)c(C#Cc2cnc3cccnn23)c1)N1N=CC[C@@H]1c1ccccc1] | (R)-N-(4-fluoro- 3-(imidazo[1,2- b]pyridazin-3- ylethynyl)phenyl)- 5-phenyl-4,5- dihydro-1H- pyrazole-1- carboxamide 2,2,2- trifluoroacetate | 425.2; 1.66 | ¹H NMR (400 MHz, DMSO-d₆) δ 9.37 (s, 1H), 8.72 (d, J = 3.4 Hz, 1H), 7.99 (dd, J = 5.3, 2.5 Hz, 1H), 7.69- 7.61 (m, 1H), 7.41 (dd, J = 9.2, 4.4 Hz, 1H), 7.35 (t, J = 7.4 Hz, 2H), 7.29- 7.16 (m, 5H), 5.33 (dd, J = 12.0, 5.6 Hz, 1H), 3.56 (dd, J = 18.3, 12.7 Hz, 1H), 2.76 (dd, |
| | | | | J = 18,7, 4.6 Hz, |
| | | | | 1H). |
| 47 | 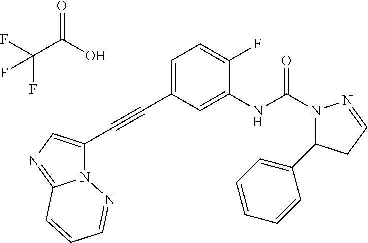 [SMILES: O=C(O)C(F)(F)F ; O=C(Nc1cc(C#Cc2cnc3cccnn23)ccc1F)N1N=CCC1c1ccccc1] | N-(2-fluoro-5- (imidazo[1,2- b]pyridazin-3- ylethynyl)phenyl)- 5-phenyl-4,5- dihydro-1H- pyrazole-1- carboxamide 2,2,2- trifluoroacetate | 425.3; 1.73 | ¹H NMR (400 MHz, DMSO-d₆) δ 8.69 (dd, J = 4.4, 1.4 Hz, 1H), 8.60 (d, J = 2.1 Hz, 1H), 8.23 (dd, J = 9.2, 1.5 Hz, 1H), 8.18 (s, 1H), 8.10 (dd, J = 7.6, 2.0 Hz, 1H), 7.40-7.30 (m, 5H), 7.29-7.19 (m, 4H), 5.34 (dd, J = 12.0, 5.5 Hz, 1H), 2.88-2.72 (m, 1H). |

### EXAMPLE 48

### Preparation of N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate

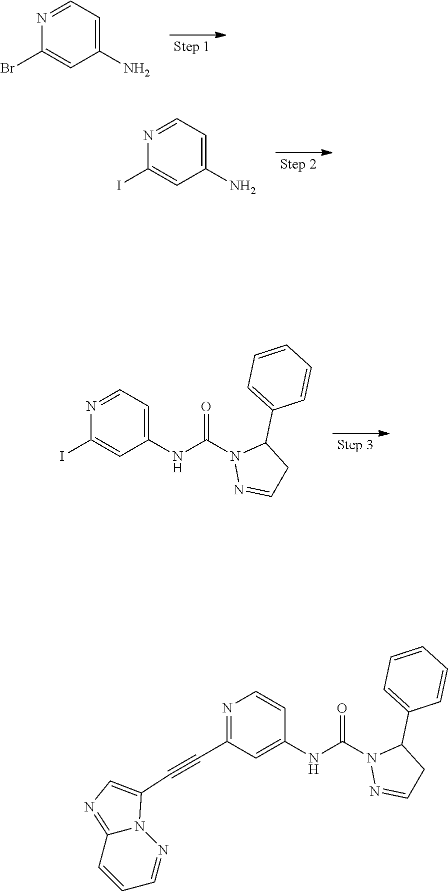 [SMILES: Nc1ccnc(Br)c1 ; Nc1ccnc(I)c1 ; O=C(Nc1ccnc(I)c1)N1N=CCC1c1ccccc1 ; O=C(Nc1ccnc(C#Cc2cnc3cccnn23)c1)N1N=CCC1c1ccccc1]

### Step 1: Preparation of 2-iodopyridin-4-amine

2-Bromopyridin-4-amine (400 mg, 2.312 mmol), CuI (881 mg, 4.62 mmol), and KI (1.92 g, 11.56 mmol) were dissolved in DMF (7.7 ml), and the gas was removed by ultrasonic treatment for 10 minutes while blowing nitrogen, followed by stirring at 130° C. overnight. The reaction mixture was filtered with celite, dissolved in excess ethyl acetate, washed with brine, and the organic layer was dried over sodium sulfate and concentrated under reduced pressure. The obtained filtrate was concentrated to give the target compound 2-iodopyridin-4-amine (105.1 mg, 83%), which was used in the next reaction without purification.

MS (m/z): 220.95[M+1]⁺, UPLC r. t. (min): 0.22

### Step 2: Preparation of N-(2-iodopyridin-4-yl)-5-phenyl-4,5-dihydro-1H-pyrazole-1-carboxamide

The compound 2-iodopyridin-4-amine obtained in step 1 above (509 mg, 2.313 mmol), 1,1′-carbonyldiimidazole (563 mg, 3.47 mmol) and triethylamine (1.62 ml, 9.25 mmol) were dissolved in DMF (7.71 ml), followed by stirring for 1 hour. 5-Phenyl-4,5-dihydro-1H-pyrazole (647 μl, 3.47 mmol) was added thereto, followed by stirring for 3 hours. Upon completion of the reaction, the reaction mixture was extracted with dichloromethane and distilled water, and the organic layers were combined. The organic layer was dried over sodium sulfate, concentrated under reduced pressure, and purified by medium pressure liquid chromatography (tetrahydrofuran/n-hexane) to give the target compound N-(2-iodopyridin-4-yl)-5-phenyl-4,5-dihydro-1H-pyrazole-1-carboxamide (180 mg, 20%) as a solid.

MS (m/z): 393.11[M+1]⁺, UPLC r. t. (min): 1.54

### Step 3: Preparation of N-(2-(imidazo[1,2-b]pyridazin-3-ylethynyl)pyridin-4-yl)-5-phenyl-4,5-dihydro-1H-pyrazol-1-carboxamide trifluoric acid salt

The compound N-(2-iodopyridin-4-yl)-5-phenyl-4,5-dihydro-1H-pyrazole-1-carboxamide obtained in step 2 above (180 mg, 0.459 mmol), the compound 3-ethynylimidazo[1,2-b]pyridazine obtained in step 2 of Example 1 (79 mg, 0.551 mmol), Pd(PPh₃)₄ (26.5 mg, 0.023 mmol), CuI (8.74 mg, 0.046 mmol) and triethylamine (256 μl, 1.836 mmol) were dissolved in acetonitrile (4.6 ml). After heating the mixture at 50° C. overnight, the temperature was lowered to room temperature to terminate the reaction. The reaction mixture was concentrated under reduced pressure using a rotary evaporator, and purified using a Prep-150 device to give the target compound N-(2-(imidazo[1,2-b]pyridazin-3-ylethynyl)pyridine-4-yl)-5-phenyl-4,5-dihydro-1H-pyrazole-1-carboxamide trifluorate (86.3 mg, 40.2%).

MS (m/z): 408.26[M+1]⁺, UPLC r. t. (min): 1.24

The compounds of Examples 49 to 52 were prepared by the similar manner to the method described in Example 48. The compound names, chemical formulas, and UPLC/NMR analysis results of the compounds of Examples 48 to 52 are summarized in Table 3 below.

TABLE 3

| | | | UPLC | |
|---|---|---|---|---|
| | | | [M + 1]⁺; | |
| | | | r.t. | |
| Example | Structure | Compound Name | (min) | ¹H NMR |
| 48 | 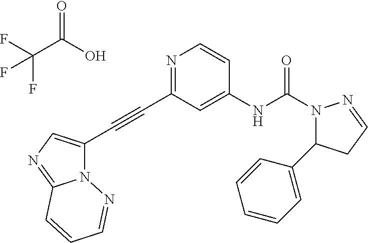 [SMILES: O=C(O)C(F)(F)F ; O=C(Nc1ccnc(C#Cc2cnc3cccnn23)c1)N1N=CCC1c1ccccc1] | N-(2- (imidazo[1,2- b]pyridazin-3- ylethynyl)pyridin- 4-yl)-5-phenyl- 4,5-dihydro-1H- pyrazole-1- carboxamide 2,2,2- trifluoroacetate | 408.3; 1.24 | ¹H NMR (400 MHz, DMSO-d₆) δ 9.77 (s, 1H), 8.71 (d, J = 3.4 Hz, 1H), 8.36 (d, J = 5.5 Hz, 1H), 8.26 (d, J = 5.9 Hz, 2H), 8.05 (s, 1H), 7.63 (d, J = 4.5 Hz, 1H), 7.42- 7.31 (m, 3H), 7.25 (dd, J = 22.2, 8.8 Hz, 4H), 5.35 (dd, J = 11.9, 5.3 Hz, 1H), 3.58 (dd, J = 18.6, 12.2 Hz, |
| | | | | 1H), 2.79 (dd, J = |
| | | | | 18.5, 4.5 Hz, 1H). |
| 49 | 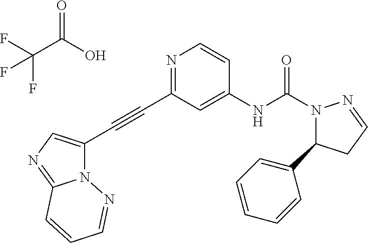 [SMILES: O=C(O)C(F)(F)F ; O=C(Nc1ccnc(C#Cc2cnc3cccnn23)c1)N1N=CC[C@H]1c1ccccc1] | (S)-N-(2- (imidazo[1,2- b]pyridazin-3- ylethynyl)pyridin- 4-yl)-5-phenyl- 4,5-dihydro-1H- pyrazole-1- carboxamide 2,2,2- trifluoroacetate | 408.3; 1.24 | ¹H NMR (400 MHz, DMSO-d₆) δ 9.77 (s, 1H), 8.71 (d, J = 3.4 Hz, 1H), 8.36 (d, J = 5.5 Hz, 1H), 8.26 (d, J = 5.9 Hz, 2H), 8.05 (s, 1H), 7.63 (d, J = 4.5 Hz, 1H), 7.42- 7.31 (m, 3H), 7.25 (dd, J = 22.2, 8.8 Hz, 4H), 5.35 (dd, J = 11.9, 5.3 Hz, 1H), 3.58 (dd, J = 18.6, 12.2 Hz, |
| | | | | 1H), 2.79 (dd, J = |
| | | | | 18.5, 4.5 Hz, 1H). |
| 50 | 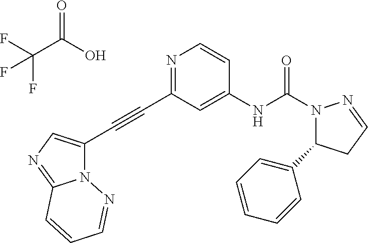 [SMILES: O=C(O)C(F)(F)F ; O=C(Nc1ccnc(C#Cc2cnc3cccnn23)c1)N1N=CC[C@@H]1c1ccccc1] | (R)-N-(2- (imidazo[1,2- b]pyridazin-3- ylethynyl)pyridin- 4-yl)-5-phenyl- 4,5-dihydro-1H- pyrazole-1- carboxamide 2,2,2- trifluoroacetate | 408.3; 1.24 | ¹H NMR (400 MHz, DMSO-d₆) δ 9.77 (s, 1H), 8.71 (d, J = 3.4 Hz, 1H), 8.36 (d, J = 5.5 Hz, 1H), 8.26 (d, J = 5.9 Hz, 2H), 8.05 (s, 1H), 7.63 (d, J = 4.5 Hz, 1H), 7.42- 7.31 (m, 3H), 7.25 (dd, J = 22.2, 8.8 Hz, 4H), 5.35 (dd, J = 11.9, 5.3 Hz, 1H), 3.58 (dd, J = 18.6, 12.2 Hz, |
| | | | | 1H), 2.79 (dd, J = |
| | | | | 18.5, 4.5 Hz, 1H). |
| 51 | 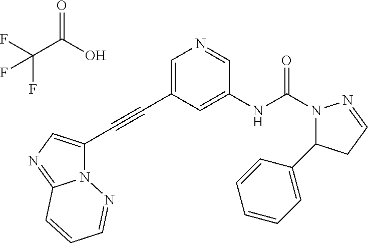 [SMILES: O=C(O)C(F)(F)F ; O=C(Nc1cncc(C#Cc2cnc3cccnn23)c1)N1N=CCC1c1ccccc1] | N-(5- (imidazo[1,2- b]pyridazin-3- ylethynyl)pyridin- 3-yl)-5-phenyl- 4,5-dihydro-1H- pyrazole-1- carboxamide 2,2,2- trifluoroacetate | 408.3; 1.40 | ¹H NMR (400 MHz, DMSO-d₆) δ 9.68 (s, 1H), 8.84 (s, 1H), 8.72 (s, 1H), 8.41 (s, 1H), 8.34 (s, 1H), 8.25 (s, 2H), 7.31 (dd, J = 55.4, 20.3 Hz, 7H), 5.41- 5.30 (m, 1H), 3.66-3.47 (m, 1H), 2.78 (d, J = 18.6 Hz, 1H). |
| 52 | 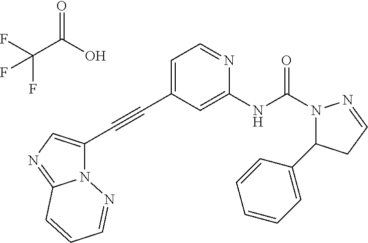 [SMILES: O=C(O)C(F)(F)F ; O=C(Nc1cc(C#Cc2cnc3cccnn23)ccn1)N1N=CCC1c1ccccc1] | N-(4- (imidazo[1,2- b]pyridazin-3- ylethynyl)pyridin- 2-yl)-5-phenyl- 4,5-dihydro-1H- pyrazole-1- carboxamide 2,2,2- trifluoroacetate | 408.3; 1.55 | ¹H NMR (400 MHz, DMSO-d₆ δ 9.18 (s, 1H), 8.75 (d, J = 4.2 Hz, 1H), 8.30 (s, 3H), 7.44 (d, J = 3.1 Hz, 1H), 7.36 (d, J = 7.3 Hz, 2H), 7.33-7.19 (m, 6H), 5.37 (dd, J = 11.7, 5.2 Hz, 1H), 3.63 (dd, J = 18.8, 12.0 Hz, 1H), 2.84 (dd, J = 18.8, 4.7 Hz, 1H). |

### EXAMPLE 53

### Preparation of (3-(imidazo[1,2-b]pyridazin-3-ylethynyl)phenyl)(5-phenyl-4,5-dihydro-1H-pyrazol-1-yl)methanone 2,2,2-trifluoroacetate

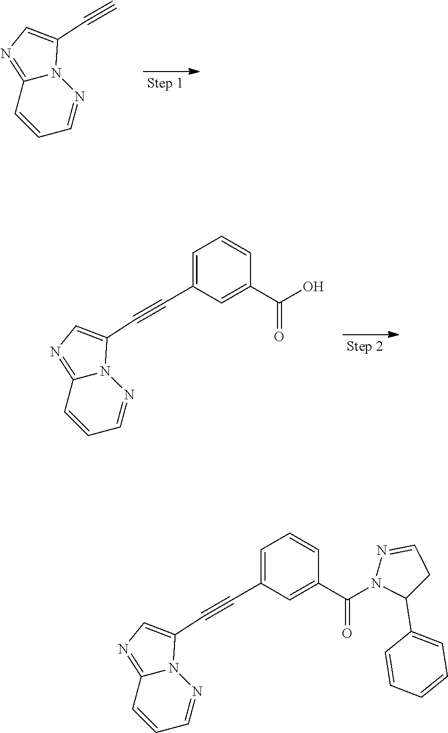 [SMILES: C#Cc1cnc2cccnn12 ; O=C(O)c1cccc(C#Cc2cnc3cccnn23)c1 ; O=C(c1cccc(C#Cc2cnc3cccnn23)c1)N1N=CCC1c1ccccc1]

### Step 1: Preparation of 3-(imidazo[1,2-b]pyridazin-3-ylethynyl)benzoic acid

The compound 3-ethynylimidazo[1,2-b]pyridazine (400 mg, 2.79 mmol) obtained in step 1 of Example 1, 3-iodobenzoic acid (760 mg, 3.07 mmol) and N,N-diisopropylethylamine (0.98 ml, 5.59 mmol) were dissolved in ethyl acetate (14 ml), and the gas was removed by ultrasonic treatment for 10 minutes while blowing nitrogen. Pd(PPh₃)₄ (161 mg, 0.14 mmol) and CuI (53 mg, 0.28 mmol) were added to the reaction mixture at 50° C. for 2 hours. Upon completion of the reaction, the reaction mixture was filtered with celite and washed with ethyl acetate. The obtained filtrate was concentrated and purified by medium pressure liquid chromatography (dichloromethane/methanol) to give the target compound 3-(imidazo[1,2-b]pyridazin-3-ylethynyl)benzoic acid (520 mg, 71%) as a solid.

MS (m/z): 234.1 [M+1]⁺, UPLC r. t. (min): 1.57

### Step 2: Preparation of (3-(imidazo[1,2-b]pyridazin-3-ylethynyl)phenyl)(5-phenyl-4,5-dihydro-1H-pyrazol-1-yl)methanone 2,2,2-trifluoroacetate

The compound 3-(imidazo[1,2-b]pyridazin-3-ylethynyl)benzoic acid obtained in step 1 above (90 mg, 0.34 mmol), COMU (176 mg, 4.1 mmol) and N,N-diisopropylethylamine (0.15 ml, 0.855 mmol) were dissolved in N,N-dimethylformamide (2 ml), followed by stirring for 30 minutes. 5-Phenyl-4,5-dihydro-1H-pyrazole (0.07 ml, 0.52 mmol) was added dropwise to the reaction mixture, followed by stirring for 1 hour. Upon completion of the reaction, the reaction mixture was extracted with ethyl acetate and brine, and the organic layers were combined. The organic layer was dried over sodium sulfate, concentrated under reduced pressure, and purified using a Prep-150 device to give the target compound (3-(imidazo[1,2-b]pyridazin-3-ylethynyl)phenyl) (5-phenyl-4,5-dihydro-1H-pyrazol-1-yl)methanone 2,2,2-trifluoroacetate (101 mg, 59%).

MS (m/z): 392.1 [M+1]⁺, UPLC r. t. (min): 1.72

The compound of Example 54 was prepared by the similar manner to the method described in Example 53. The compound names, chemical formulas, and UPLC/NMR N analysis results of the compounds of Examples 53 and 54 are summarized in Table 4 below.

TABLE 4

| | | | MASS | UPLC |
|---|---|---|---|---|
| Example | Structure | Compound Name | [M + 1]⁺ | r.t. (min) |
| 53 | 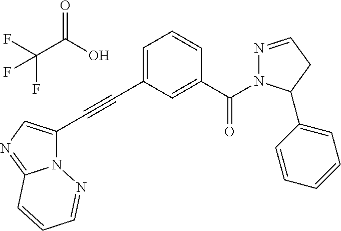 | (3-(imidazo[1,2- b]pyridazin-3- ylethynyl)phenyl) (5-phenyl-4,5- dihydro-1H- pyrazol-1- yl)methanone 2,2,2- trifluoroacetate | 392.1 | 1.72 |
| 54 | 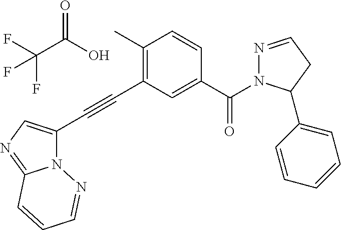 | (3-(imidazo[1,2- b]pyridazin-3- ylethynyl)-4- methylphenyl)(5- phenyl-4,5- dihydro-1H- pyrazol-1- yl)methanone 2,2,2- trifluoroacetate | 406.1 | 1.8 |

### EXAMPLE 55

### Preparation of N-(3-(imidazo[1,2-a]pyridin-3-ylethynyl)phenyl)-5-phenyl-4,5-dihydro-1H-pyrazole-1-carboxamide

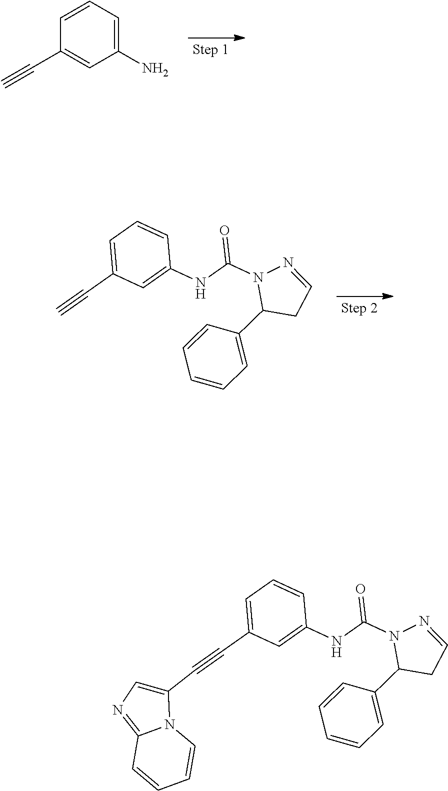

### Step 1: Preparation of N-(3-ethynylphenyl)-3-phenyl-3,4-dihydropyrazole-2-carboxamide

3-Ethynylaniline (100 g, 853.62 mmol) and DIEA (220.65 g, 1.71 mol, 297.37 mL) were added, to which acetonitrile (1000 mL) was added and diluted, and then DSC (240.54 g, 938.99 mmol) was added slowly at 0° C. After reacting the mixture for 1 hour, 5-phenyl-4,5-dihydro-1H-pyrazole (162.23 g, 1.11 mol) and DIEA (220.65 g, 1.71 mol, 297.37 mL) were added thereto, followed by reaction for 10 hours. The reaction mixture was concentrated under reduced pressure using a rotary evaporator, extracted with ethyl acetate and brine, and the organic layers were combined. The organic layer was dried over sodium sulfate, concentrated under reduced pressure to give the target compound N-(3-ethynylphenyl)-5-phenyl-4,5-dihydro-1H-pyrazole-1-carboxamide (270 g, 54.7%), which was used in the next reaction without purification.

¹H NMR (400 MHz, CDCl₃) δ 7.92 (s, 1H), 7.57 (t, J=1.6 Hz, 1H), 7.44-7.38 (m, 1H), 7.30-7.24 (m, 2H), 7.22-7.15 (m, 4H), 7.13-7.11 (t, J=8 Hz, 1H), 7.08-7.04 (m, 1H), 6.81 (t, J=1.7 Hz, 1H), 5.30 (m, 1H), 3.44 (m, 1H), 2.82 (m, 1H)

### Step 2: Preparation of N-(3-(imidazo[1,2-a]pyridin-3-ylethynyl)phenyl)-5-phenyl-4,5-dihydro-1H-pyrazole-1-carboxamide

The compound N-(3-ethynylphenyl)-5-phenyl-4,5-dihydro-1H-pyrazole-1-carboxamide (200 mg, 691.25 pmol) obtained in step 1 above and 3-bromoimidazo[1,2-a]pyridine (204.30 mg, 1.04 mmol) were dissolved in DMF (5 mL), and the gas was removed by ultrasonic treatment for 5 minutes while blowing nitrogen. Pd(PPh₃) ₄ (239.63 mg, 207.38 pmol), CuI (39.49 mg, 207.38 μmol) and TEA (481.07 μL, 3.46 mmol) were added to the reaction mixture, followed by reaction at 65° C. for 6 hours. Upon completion of the reaction, the temperature was lowered to room temperature. Distilled water and ethyl acetate were added to the reaction mixture, and the organic layers were combined by extraction with ethyl acetate and distilled water. The organic layer was dried over sodium sulfate, concentrated under reduced pressure, and purified using a Prep-150 device to give the target compound N-(3-(imidazo[1,2-a]pyridin-3-ylethynyl)phenyl)-5-phenyl-4,5-dihydro-1H-pyrazole-1-carboxamide (22 mg, 7.85%).

MS: m/z 406.3 [M+H]⁺

¹H NMR (400 MHz, DMSO-d₆) δ 9.23 (s, 1H), 8.57 (d, J=6.6 Hz, 1H), 8.01 (s, 1H), 7.96 (s, 1H), 7.72 (d, J=9.0 Hz, 1H), 7.63 (m, 1H), 7.46-7.40 (m, 1H), 7.38-7.33 (m, 2H), 7.31 (m, 1H), 7.29-7.19 (m, 5H), 7.14 (t, J=6.8 Hz, 1H), 5.34 (m, 1H), 3.62-3.51 (m, 1H), 2.81-2.75 (m, 1H)

The compounds of Examples 54 to 58 were prepared by the similar manner to the method described in Example 55. The compound names, chemical formulas, and UPLC/¹H-NMR analysis results of the compounds of Examples 55 and 58 are summarized in Table 5 below.

TABLE 5

| | | | UPLC | ¹H NMR |
|---|---|---|---|---|
| Example | Structure | Compound Name | [M + 1]⁺ | (400 MHz, DMSO-d₆) |
| 55 | 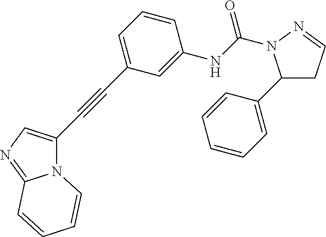 [SMILES: O=C(Nc1cccc(C#Cc2cnc3ccccn23)c1)N1N=CCC1c1ccccc1] | N-(3- (imidazo[1,2- a]pyridin-3- ylethynyl)phenyl)- 5-phenyl- 4,5-dihydro-1H- pyrazole-1- carboxamide | 406.3 | δ 9.23 (s, 1H), 8.57 (d, J = 6.6 Hz, 1H), 8.01 (s, 1H), 7.96 (s, 1H), 7.72 (d, J = 9.0 Hz, 1H), 7.63 (m, 1H), 7.46-7.40 (m, 1H), 7.38-7.33 (m, 2H), 7.31 (m, 1H), 7.29-7.19 (m, 5H), 7.14 (t, J = 6.8 Hz, 1H), 5.34 (m, 1H), 3,62-3.51 (m, 1H), 2,81-2.75 (m, 1H) |
| 56 | 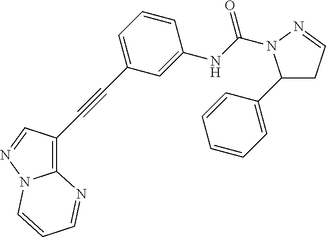 [SMILES: O=C(Nc1cccc(C#Cc2cnn3cccnc23)c1)N1N=CCC1c1ccccc1] | 5-phenyl-N-(3- (pyrazolo[1,5- a]pyrimidin-3- ylethynyl)phenyl)- 4,5-dihydro- 1H-pyrazole-1- carboxamide | 407.2 | δ 9.29-9.14 (m, 2H), 8.68 (dd, J = 1.6, 4.0 Hz, 1H), 8.51 (s, 1H), 7.89 (d, J = 1.6 Hz, 1H), 7.61-7.52 (m, 1H), 7.39-7.32 (m, 2H), 7.30-7.16 (m, 6H), 7.12 (d, J = 7.7 Hz, 1H), 5.33 (m, 1H), 3.57 (m, 1H), 2.76 (m, 1H) |
| 57 | 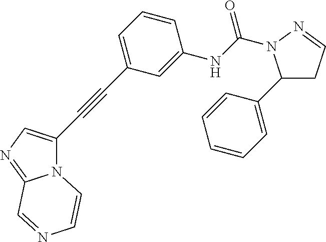 [SMILES: O=C(Nc1cccc(C#Cc2cnc3cnccn23)c1)N1N=CCC1c1ccccc1] | N-(3- (imidazo[1,2- a]pyrazin-3- ylethynyl)phenyl)- 5-phenyl- 4,5-dihydro-1H- pyrazole-1- carboxamide | 407.2 | δ 9.26 (s, 1H), 9.19 (d, J = 1.3 Hz, 1H), 8.66 (dd, J = 1.4, 4.4 Hz, 1H), 8.22 (s, 1H), 8.10 (d, J = 3.2 Hz, 1H), 8.01 (s, 1H), 7.65 (m, 1H), 7.39-7.24 (m, 5H), 7.22 (m, 3H), 5.33 (m, 1H), 3.65- 3.51 (m, 1H), 2.77 (m, 1H) |
| 58 | 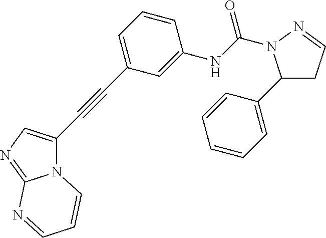 [SMILES: O=C(Nc1cccc(C#Cc2cnc3ncccn23)c1)N1N=CCC1c1ccccc1] | N-(3- (imidazo[1,2- a]pyrimidin-3- ylethynyl)phenyl)- 5-phenyl- 4,5-dihydro-1H- pyrazole-1- carboxamide | 407.1 | δ 9.24 (s, 1H), 9.03 (dd, J = 2.0, 6.6 Hz, 1H), 8.68 (m, 1H), 8.17 (s, 1H), 7.99 (s, 1H), 7.63 (m, 1H), 7.39-7.30 (m, 3H), 7.29-7.18 (m, 6H), 5.33 (m, 1H), 3.57 (m, 1H), 2.85-2.69 (m, 1H) |

### EXAMPLE 59

### Preparation of N-(3-((8-aminoimidazo[1,2-a]pyridin-3-yl)ethynyl)phenyl)-5-phenyl-4,5-dihydro-1H-pyrazole-1-carboxamide hydrochloride

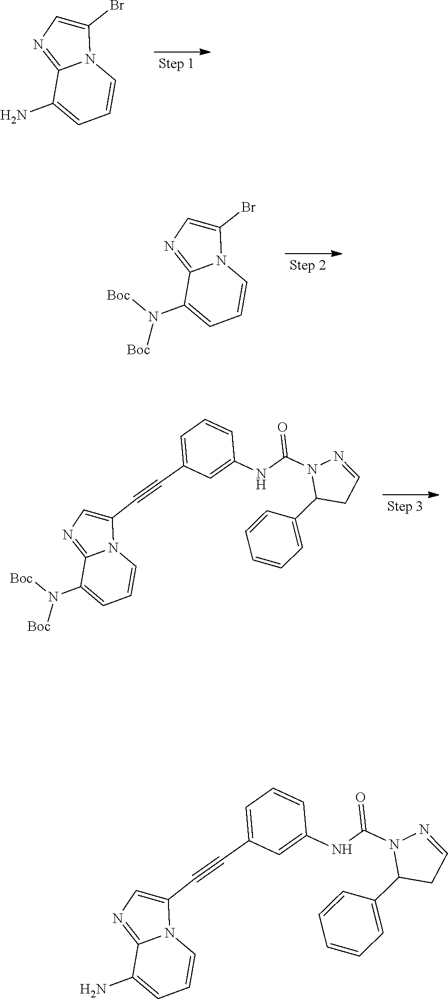 [SMILES: Nc1cccn2c(Br)cnc12 ; CC(C)(C)OC(=O)N(C(=O)OC(C)(C)C)c1cccn2c(Br)cnc12 ; CC(C)(C)OC(=O)N(C(=O)OC(C)(C)C)c1cccn2c(C#Cc3cccc(NC(=O)N4N=CCC4c4ccccc4)c3)cnc12 ; Nc1cccn2c(C#Cc3cccc(NC(=O)N4N=CCC4c4ccccc4)c3)cnc12]

### Step 1: Preparation of tert-butyl N-(3-bromoimidazo[1,2-a]pyridin-8-yl)-N-tert-butoxycarbonyl-carbamate

3-Bromoimidazo[1,2-a]pyridin-8-amine (1.8 g, 8.49 mmol) was dissolved in DCM (40 mL), to which DMAP (2.59 g, 21.22 mmol) and tert-butoxycarbonyl tert-butylcarbonate (4.88 mL, 21.22 mmol) were added, followed by reaction at 30° C. for 12 hours. Upon completion of the reaction, the temperature was lowered to room temperature, and the reaction mixture was concentrated under reduced pressure using a rotary evaporator and purified by medium pressure liquid chromatography (petronium ether/ethyl acetate) to give the target compound tert-butyl N-(3-bromoimidazo[1,2-a]pyridin-8-yl)-N-tert-butoxycarbonyl-carbamate (3.2 g, 91.4%).

MS: m/z 412.2 [M+H]⁺

### Step 2: Preparation of tert-butyl N-tert-butoxycarbonyl-N-[3-[2-[3-[(3-phenyl-3,4-dihydropyrazole-2-carbonyl)amino]phenyl]ethynyl]imidazo[1,2-a]pyridin-8-yl]carbamate

The compound tert-butyl N-(3-bromoimidazo[1,2-a]pyridin-8-yl)-N-tert-butoxycarbonyl-carbamate (2 g, 4.85 mmol) obtained in step 1 above and the compound N-(3--ethynylphenyl)-3-phenyl-3,4-dihydropyrazole-2-carboxamide (2.81 g, 9.70 mmol) obtained in step 1 of Example 1 were dissolved in DMF (40 mL), and the gas was removed by ultrasonic treatment for 5 minutes while blowing nitrogen. Pd(PPh₃)₄ (1.68 g, 1.46 mmol) and TEA (3.38 mL, 24.26 mmol) were added thereto, followed by reaction at 70° C. for 12 hours. Upon completion of the reaction, the temperature was lowered to room temperature, and the reaction mixture was extracted with dichloromethane by adding distilled water. The organic layer was extracted with dichloromethane and brine, and the organic layers were combined. The organic layer was dried over sodium sulfate, concentrated under reduced pressure using a rotary evaporator, and purified by medium pressure liquid chromatography (petronium ether/ethyl acetate) to give the target compound tert-butyl N-tert-butoxycarbonyl-N-[3-[2-[3-[(3-phenyl-3,4-dihydropyrazole-2-carbonyl)amino]phenyl]ethynyl]imidazo[1,2-a]pyridin-8-yl]carbamate (1.7 g, 56.5%).

MS: m/z 621.3 [M+H]⁺

### Step 3: Preparation of N-(3-((8-aminoimidazo[1,2-a]pyridin-3-yl)ethynyl)phenyl)-5-phenyl-4,5-dihydro-1H-pyrazole-1-carboxamide hydrochloride

The compound tert-butyl N-tert-butoxycarbonyl-N-[3-[2-[3-[(3-phenyl-3,4-dihydropyrazole-2-carbonyl)amino]phenyl]ethynyl]imidazo[1,2-a]pyridin-8-yl]carbamate (2.0 g, 3.22 mmol) obtained in step 2 above was dissolved in ethyl acetate (40 mL), to which HCl/in EtOAc (4 M , 8.06 mL) was added slowly. After reacting the mixture for 2 hours, the reaction mixture was concentrated under reduced pressure using a rotary evaporator to give the target compound N-(3-((8-aminoimidazo[1,2-a]pyridin-3-yl)ethynyl)phenyl)-5-phenyl-4,5-dihydro-1H-pyrazole-1-carboxamide hydrochloride (1.6 g, 35.9%) as a yellow solid.

MS: m/z 421.3 [M+H]⁺

¹H NMR (400 MHz, DMSO-d₆) δ 9.33-9.25 (m, 1H), 8.39 (m, 1H), 8.21-7.91 (m, 2H), 7.76-7.61 (m, 1H), 7.42-7.19 (m, 8H), 7.48-7.05 (m, 1H), 6.97-6.64 (m, 1H), 5.33 (m, J=5.4, 11.6 Hz, 1H), 3.61 (m, 1H), 2.76 (m, 1H)

### EXAMPLE 60

### Preparation of N-(3-((8-acetamidoimidazo[1,2-a]pyridin-3-yl)ethynyl)phenyl)-5-phenyl-4,5-dihydro-1H-pyrazole-1-carboxamide

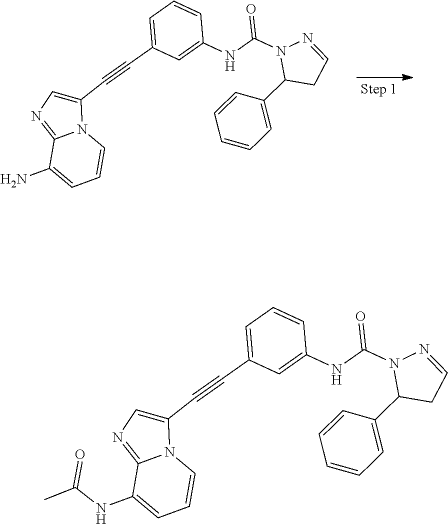 [SMILES: Nc1cccn2c(C#Cc3cccc(NC(=O)N4N=CCC4c4ccccc4)c3)cnc12 ; CC(=O)Nc1cccn2c(C#Cc3cccc(NC(=O)N4N=CCC4c4ccccc4)c3)cnc12]

### Step 1: Preparation of N-(3-((8-acetamidoimidazo[1,2-a]pyridin-3-yl)ethynyl)phenyl)-5-phenyl-4,5-dihydro-1H-pyrazole-1-carboxamide

The compound N-(3-((8-aminoimidazo[1,2-a]pyridin-3-yl)ethynyl)phenyl)-5-phenyl-4,5-dihydro-1H-pyrazole-1-carboxamide hydrochloride (0.2 g, 144.44 μmol) obtained in step 3 of Example 59 was dissolved in DCM (5 mL), to which TEA (60.31 μL, 433.33 umol) and acetylacetate (67.64 μL, 722.22 nmol) were added, followed by reaction for 12 hours. Upon completion of the reaction, the reaction mixture was concentrated under reduced pressure using a rotary evaporator and then purified using a Prep-150 device to give the target compound N-(3-((8-acetamidoimidazo[1,2-a]pyridin)-3-yl)ethynyl)phenyl)-5-phenyl-4,5-dihydro-1H-pyrazole-1-carboxamide (37.1 mg, 54.8%) as a yellow solid.

MS: m/z 463.2 [M+H]⁺

¹H NMR (400 MHz, DMSO-d₆) δ 10.15 (s, 1H), 9.26 (s, 1H), 8.28 (dd, J=0.9, 6.7 Hz, 1H), 8.12 (d, J=7.4 Hz, 1H), 8.00 (s, 1H), 7.96 (t, J=1.7 Hz, 1H), 7.67-7.59 (m, 1H), 7.39-7.29 (m, 3H), 7.29-7.24 (m, 2H), 7.24-7.19 (m, 3H), 7.11-7.06 (m, 1H), 5.33 (m, 1H), 3.57 (m, 1H), 2.87-2.70 (m, 1H), 2.22 (s, 3H)

### EXAMPLE 61

### Preparation of N-(3-((8-(3-methylureido)imidazo[1,2-a]pyridin-3-yl)ethynyl)phenyl)-5-phenyl-4,5-dihydro-1H-pyrazole-1-carboxamide 2,2,2-trifluoroacetate

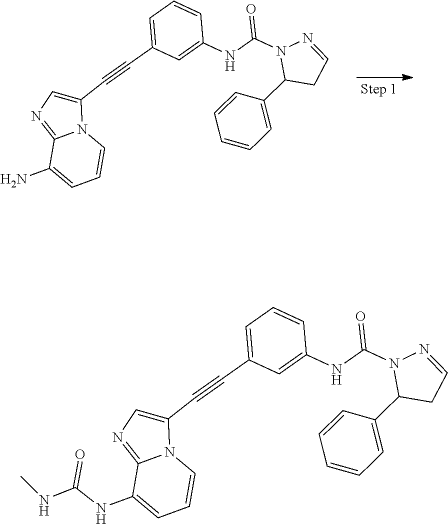 [SMILES: Nc1cccn2c(C#Cc3cccc(NC(=O)N4N=CCC4c4ccccc4)c3)cnc12 ; CNC(=O)Nc1cccn2c(C#Cc3cccc(NC(=O)N4N=CCC4c4ccccc4)c3)cnc12]

### Step 1: Preparation of N-(3-((8-(3-methylureido)imidazo[1,2-a]pyridin-3-yl)ethynyl)phenyl)-5-phenyl-4,5-dihydro-1H-pyrazole-1-carboxamide trifluoroacetate

The compound N-(3-((8-aminoimidazo[1,2-a]pyridin-3-yl)ethynyl)phenyl)-5-phenyl-4,5-dihydro-1H-pyrazole-1-carboxamide hydrochloride (0.2 g, 144.44 umol) obtained in step 3 of Example 59 was dissolved in pyridine (4 mL), to which N-methylcarbamoyl chloride (3.90 μL, 722.22 μmol) was added, followed by reaction at 50° C. for 12 hours. Upon completion of the reaction, the temperature was lowered to room temperature, and the reaction mixture was concentrated under reduced pressure using a rotary evaporator, and purified using a Prep-150 device to give the target compound N-(3-((8-(3-methylureido)imidazo)[1,2-a]pyridin-3-yl)ethynyl)phenyl)-5-phenyl-4,5-dihydro-1H-pyrazole-1-carboxamide trifluoroacetate (38.4 mg, 44.9%) as a yellow solid.

MS: m/z 478.1 [M+H]⁺

¹H NMR (400 MHz, DMSO-d₆) δ 9.25 (s, 1H), 8.92 (s, 1H), 8.13 (d, J=6.6 Hz, 1H), 8.00 (d, J=7.0 Hz, 1H), 7.97 (d, J=5.0 Hz, 2H), 7.63 (m, 1H), 7.34 (m, 3H), 7.29-7.19 (m, 5H), 7.05 (t, J=7.2 Hz, 2H), 5.34 (m, 1H), 3.57 (m, 1H), 2.76 (m, 1H)

The compounds of Examples 59 to 61 were prepared by the method described above, and the compounds of Examples 62 to 64 were prepared by the similar manner to the method described in Example 61. The compound names, chemical formulas, and UPLC/¹H-NMR analysis results of the compounds of Examples 59 to 64 are summarized in Table 6 below.

TABLE 6

| | | | UPLC | ¹H NMR |
|---|---|---|---|---|
| Example | Structure | Compound Name | [M + 1]⁺ | (400 MHz, DMSO-d₆) |
| 59 | 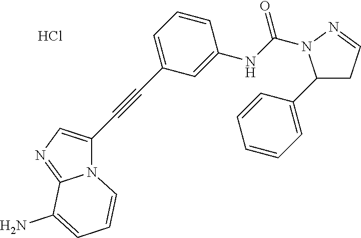 [SMILES: Nc1cccn2c(C#Cc3cccc(NC(=O)N4N=CCC4c4ccccc4)c3)cnc12 ; Cl] | N-(3-((8- aminoimidazo[1, 2-a]pyridin-3- yl)ethynyl) phenyl)-5-phenyl- 4,5-dihydro-1H- pyrazole-1- carboxamide hydrochloride | 421.3 | δ 9.33-9.25 (m, 1H), 8.39 (m, 1H), 8.21-7.91 (m, 2H), 7.76-7.61 (m, 1H), 7.42- 7.19 (m, 8H), 7.48- 7.05 (m, 1H), 6.97-6.64 (m, 1H), 5.33 (m, J = 5.4, 11.6 Hz, 1H), 3.61 (m, 1H), 2.76 (m, 1H) |
| 60 | 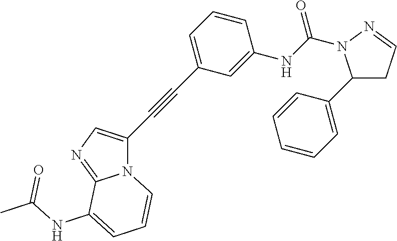 [SMILES: CC(=O)Nc1cccn2c(C#Cc3cccc(NC(=O)N4N=CCC4c4ccccc4)c3)cnc12] | N-(3-((8- acetamidoimidazo [1,2- a]pyridin-3- yl)ethynyl) phenyl)-5-phenyl- 4,5-dihydro-1H- pyrazole-1- carboxamide | 463.2 | δ 10.15 (s, 1H), 9.26 (s, 1H), 8.28 (dd, J = 0.9, 6.7 Hz, 1H), 8.12 (d, J = 7.4 Hz, 1H), 8.00 (s, 1H), 7.96 (t, J = 1.7 Hz, 1H), 7.67- 7.59 (m, 1H), 7.39-7.29 (m, 3H), 7.29-7.24 (m, 2H), 7.24- 7.19 (m, 3H), 7.11- 7.06 (m, 1H), 5.33 (m, 1H), 3.57 (m, 1H), 2.87- |
| | | | | 2.70 (m, 1H), 2.22 |
| | | | | (s, 3H) |
| 61 | 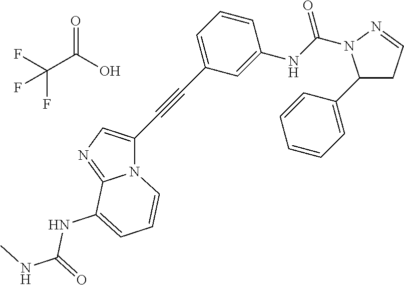 [SMILES: CNC(=O)Nc1cccn2c(C#Cc3cccc(NC(=O)N4N=CCC4c4ccccc4)c3)cnc12 ; O=C(O)C(F)(F)F] | N-(3-((8-(3- methylureido) imidazo[1,2- a]pyridin-3- yl)ethynyl) phenyl)-5-phenyl- 4,5-dihydro-1H- pyrazole-1- carboxamide 2,2,2- trifluoroacetate | 478.1 | δ 9.25 (s, 1H), 8.92 (s, 1H), 8.13 (d, J = 6.6 Hz, 1H), 8.00 (d, J = 7.0 Hz, 1H), 7.97 (d, J = 5.0 Hz, 2H), 7.63 (m, 1H), 7.34 (m, 3H), 7.29- 7.19 (m, 5H), 7.05 (t, J = 7.2 Hz, 2H), 5.34 (m, 1H), 3.57 (m, 1H), 2.76 (m, 1H) |
| 62 | 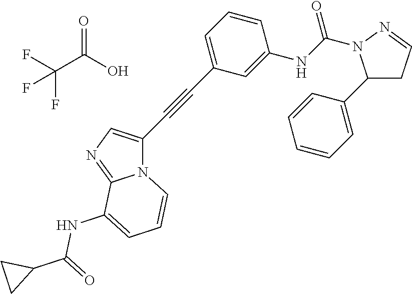 [SMILES: O=C(Nc1cccn2c(C#Cc3cccc(NC(=O)N4N=CCC4c4ccccc4)c3)cnc12)C1CC1 ; O=C(O)C(F)(F)F] | N-(3-((8- (cyclopropane- carboxamido) imidazo[1,2- a]pyridin-3- yl)ethynyl) phenyl)-5-phenyl- 4,5-dihydro-1H- pyrazole-1- carboxamide 2,2,2- trifluoroacetate | 489.3 | δ 10.44 (s, 1H), 9.26 (s, 1H), 8.28 (dd, J = 1.0, 6.8 Hz, 1H), 8.12- 8.07 (m, 1H), 8.02 (s, 1H), 7.96 (t, J = 1.6 Hz, 1H), 7.66- 7.58 (m, 1H), 7.39-7.29 (m, 3H), 7.29-7.19 (m, 5H), 7.11- 7.05 (m, 1H), 5.34 (m, 1H), 3.61-3.53 (m, 1H), 2.80-2.73 (m, 1H), 2.38- 2.29 (m, 2H), 0.90- 0.76 (m, 4H) |
| 63 | 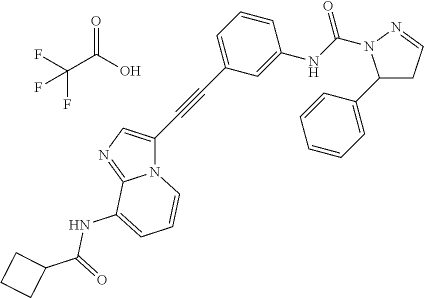 [SMILES: O=C(Nc1cccn2c(C#Cc3cccc(NC(=O)N4N=CCC4c4ccccc4)c3)cnc12)C1CCC1 ; O=C(O)C(F)(F)F] | N-(3-((8- (cyclobutane- carboxamido) imidazo[1,2- a]pyridin-3- yl)ethynyl) phenyl)-5-phenyl- 4,5-dihydro-1H- pyrazole-1- carboxamide 2,2,2- trifluoroacetate | 503.3 | δ 9.84 (s, 1H), 9.26 (s, 1H), 8.28 (dd, J = 1.0, 6.6 Hz, 1H), 8.15 (d, J = 7.4 Hz, 1H), 7.99 (s, 1H), 7.96 (t, J = 1.8 Hz, 1H), 7.66- 7.59 (m, 1H), 7.38-7.29 (m, 3H), 7.29-7.19 (m, 5H), 7.12- 7.07 (m, 1H), 5.33 (m, 1H), 3.66- 3.53 (m, 2H), 2.76 (m, 1H), 2.30- 2.10 (m, 4H), 2.02- 1.90 (m, 1H), 1.88-1.77 (m, 1H) |
| 64 | 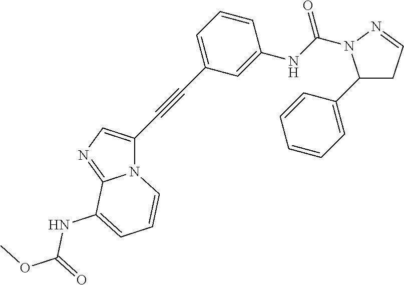 [SMILES: COC(=O)Nc1cccn2c(C#Cc3cccc(NC(=O)N4N=CCC4c4ccccc4)c3)cnc12] | methyl (3-((3- (5-phenyl-4,5- dihydro-1H- pyrazole-1- carboxamido) phenyl)ethynyl) imidazo[1,2- a]pyridin-8- yl)carbamate | 479.2 | ¹H NMR (400 MHz, DMSO-d₆) δ = 9.45 (s, 1H), 9.25 (s, 1H), 8.34 (d, J = 6.0 Hz, 1H), 8.07 (s, 1H), 7.98 (s, 1H), 7.81 (d, J = 7.6 Hz, 1H), 7.66-7.60 (m, 2H), 7.38- 7.30 (m, 3H), 7.29- 7.24 (m, 2H), 7.24-7.19 (m, 3H), 7.17 (t, J = 7.2 Hz, 1H), 5.36- 5.31 (m, 1H), 3.74 (s, 3H), 3.63- 3.61 (m, 1H), 2.77 (m, 1H) |

### EXAMPLE 65

### Preparation of N-(3-((8-((4-(methylsulfonyl)phenyl)amino)imidazo[1,2-a]pyridin-3-yl)ethynyl)phenyl)-5-phenyl-4,5-dihydro-1H-pyrazole-1-carboxamide 2,2,2-trifluoroacetate

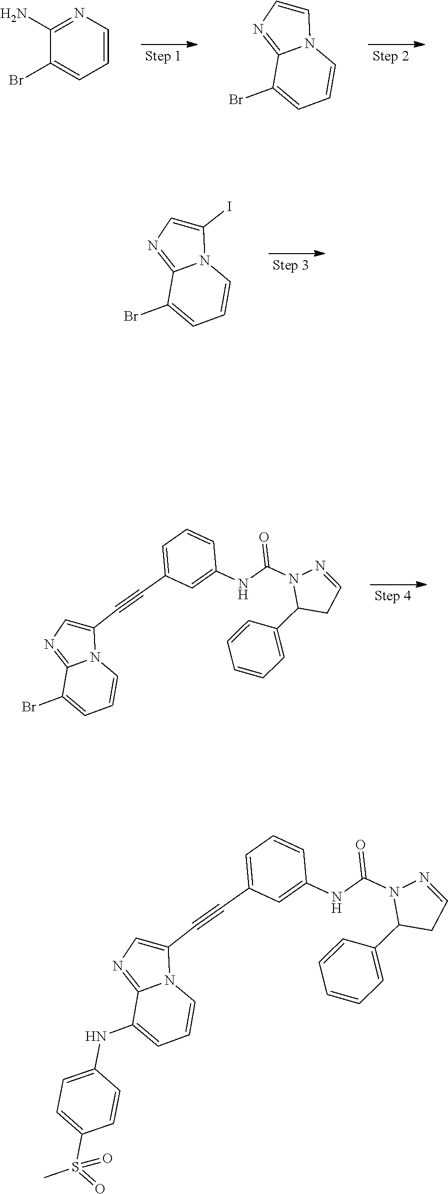 [SMILES: Nc1ncccc1Br ; Brc1cccn2ccnc12 ; Brc1cccn2c(I)cnc12 ; O=C(Nc1cccc(C#Cc2cnc3c(Br)cccn23)c1)N1N=CCC1c1ccccc1 ; CS(=O)(=O)c1ccc(Nc2cccn3c(C#Cc4cccc(NC(=O)N5N=CCC5c5ccccc5)c4)cnc23)cc1]

### Step 1: Preparation of 8-bromoimidazo[1,2-a]pyridine

2-Chloroacetaldehyde (7.44 mL, 46.24 mmol) was dissolved in H₂O (5 mL). While heating the mixture at 60° C., EtOH (20 mL) containing 3-bromopyridin-2-amine (2.0 g, 11.56 mmol) dissolved therein was added thereto, followed by stirring. The temperature of the reaction mixture was raised to 80° C., followed by reaction for 10 hours. Upon completion of the reaction, the temperature was lowered to room temperature, and the reaction mixture was concentrated under reduced pressure using a rotary evaporator. The concentrate was extracted with ethyl acetate, NaHCO₃ and brine, the pH was adjusted to 8-9, and the organic layers were combined. The organic layer was dried over sodium sulfate and then concentrated under reduced pressure to give the target compound 8-bromoimidazo[1,2-a]pyridine (2.2 g, 96.6%) as a brown solid, which was used in the next reaction without purification.

¹H NMR (400 MHz, DMSO-d₆) δ 8.60 (dd, J=0.6, 6.6 Hz, 1H), 8.10 (d, J=1.0 Hz, 1H), 7.64 (s, 1H), 7.58 (d, J=7.2 Hz, 1H), 6.83 (t, J=7.0 Hz, 1H)

### Step 2: Preparation of 8-bromo-3-iodo-imidazo[1,2-a]pyridine

The compound 8-bromoimidazo[1,2-a]pyridine (1 g, 5.08 mmol) obtained in step 1 above was dissolved in DMF (10 mL), to which NIS (1.37 g, 6.09 mmol) was added, followed by reaction for 12 hours. Upon completion of the reaction, the temperature was lowered to room temperature, and H₂O (50 mL) was added to the reaction mixture. The resulting solid was filtered and dissolved in THF (20 mL) and H₂O (40 mL). The mixture was extracted with ethyl acetate, distilled water and brine, and the organic layers were combined. The organic layer was dried over sodium sulfate and concentrated under reduced pressure to give the target compound 8-bromo-3-iodo-imidazo[1,2-a]pyridine (1.0 g, 61.0%) as a brown solid, which was used in the next reaction without purification.

MS: m/z 324.9 [M+H]⁺

¹H NMR (400 MHz, DMSO-d₆) δ 8.38 (dd, J=0.6, 6.7 Hz, 1H), 7.80 (s, 1H), 7.75-7.63 (m, 1H), 7.00 (t, J=7.1 Hz, 1H)

### Step 3: Preparation of —N-[3-[2-(8-bromoimidazo[1,2-a]pyridin-3-yl)ethynyl]phenyl]-3-phenyl-3,4-dihydropyrazole-2-carboxamide

The compound 8-bromo-3-iodo-imidazo[1,2-a]pyridine (800 mg, 2.48 mmol) obtained in step 2 above and the compound N-(3-ethynylphenyl)-3-phenyl-3,4-dihydropyrazole-2-carboxamide (788.45 mg, 2.73 mmol) obtained in step 1 of Example 1 were dissolved in DMF (10 mL), and the gas was removed by ultrasonic treatment for 5 minutes while blowing nitrogen. CuI (235.90 mg, 1.24 mmol), ethyl acetate (1.72 mL, 12.39 mmol) and Pd(PPh₃)₄ (858.81 mg, 743.20 umol) were added thereto, followed by reaction at 70° C. for 6 hours. Upon completion of the reaction, the temperature was lowered to room temperature, and H₂O (10 mL) and ethyl acetate (50 mL) were added to the reaction mixture, followed by extraction with ethyl acetate and brine, and the organic layers were combined. The organic layer was dried over sodium sulfate, concentrated under reduced pressure, and purified by medium pressure liquid chromatography (petronium ether/ethyl acetate) to give the target compound N-[3-[2-(8-bromoimidazo[1,2-a]pyridin-3-ethynyl]phenyl]-3-phenyl-3,4-dihydropyrazole-2-carboxamide (1.1 g, 91.7%) as a yellow solid.

MS: m/z 486.1 [M+H]⁺

¹H NMR (400 MHz, DMSO-d₆) δ 9.25 (s, 1H), 8.61 (d, J=6.6 Hz, 1H), 8.07 (s, 1H), 8.01-7.94 (m, 1H), 7.78 (d, J=7.3 Hz, 1H), 7.67-7.59 (m, 2H), 7.38-7.33 (m, 2H), 7.29-7.17 (m, 5H), 7.05 (t, J=7.0 Hz, 1H), 5.33 (m, 1H), 3.57 (m, 1H), 2.82-2.72 (m, 1H)

### Step 4: Preparation of N-(3-((8-((4-(methylsulfonyl)phenyl)amino)imidazo[1,2-a]pyridin-3-yl)ethynyl)phenyl)-5-phenyl-4,5-dihydro-1H-pyrazole-1-carboxamide trifluoroacetate

The compound N-[3-[2-(8-bromoimidazo[1,2-a]pyridin-3-yl)ethynyl]phenyl]-3-phenyl-3,4-dihydropyrazole-2-carboxamide (200 mg, 412.93 μmol) obtained in step 3 above and 4-methylsulfonylaniline (106.05 mg, 619.39 umol) were dissolved in 1,4-dioxane (2 mL), and the gas was removed by ultrasonic treatment for 5 minutes while blowing nitrogen. KOAc (121.58 mg, 1.24 mmol), Pd₂(dba)₃ (75.62 mg, 82.59 pmol) and t-BuXphos (52.60 mg, 123.88 umol) were added thereto, followed by reaction at 100° C. for 10 hours. Upon completion of the reaction, the temperature was lowered to room temperature, and the reaction mixture was concentrated under reduced pressure using a rotary evaporator, and purified by medium pressure liquid chromatography (petronium ether/ethyl acetate), and then purified again using a Prep-150 device to give the target compound N-(3-((8-((4-(methylsulfonyl)phenyl)amino)imidazo[1,2-a]pyridin-3-yl)ethynyl)phenyl)-5-phenyl-4,5-dihydro-1H-pyrazole-1-carboxamide trifluoroacetate (8 mg, 3.2%) as a brown solid.

MS: m/z 575.2 [M+H]⁺

¹H NMR (400 MHz, DMSO-d₆) δ 9.27 (s, 1H), 9.23 (s, IH), 8.26 (d, J=6.6 Hz, 1H), 8.04 (s, 1H), 7.98 (s, 1H), 7.78 (d, J=8.7 Hz, 2H), 7.64 (d, J=8.0 Hz, 1H), 7.43 (d, J=8.8 Hz, 2H), 7.39-7.30 (m, 4H), 7.30-7.24 (m, 2H), 7.22 (d, J=7.6 Hz, 3H), 7.11 (t, J=7.0 Hz, 1H), 5.34 (m, 1H), 3.58-3.57 (m, IH), 3.16 (m, 3H), 2.77 (m, 1H)

The compounds of Examples 66 to 68 were prepared by the similar manner to the method described in Example 65. The compound names, chemical formulas, and UPLC/¹H-NMR analysis results of the compounds of Examples 65 to 68 are summarized in Table 7 below.

TABLE 7

| | | | UPLC | ¹H NMR |
|---|---|---|---|---|
| Example | Structure | Compound Name | [M + 1]⁺ | (400 MHz, DMSO-d₆) |
| 65 | 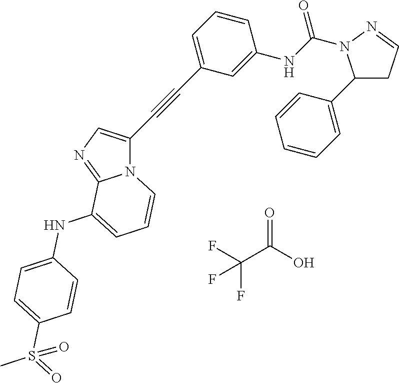 [SMILES: CS(=O)(=O)c1ccc(Nc2cccn3c(C#Cc4cccc(NC(=O)N5N=CCC5c5ccccc5)c4)cnc23)cc1 ; O=C(O)C(F)(F)F] | N-(3-((8-((4- (methylsulfonyl) phenyl)amino) imidazo[1,2- a]pyridin-3- yl)ethynyl)phenyl)- 5-phenyl-4,5- dihydro-1H- pyrazole-1- carboxamide 2,2,2- trifluoroacetate | 575.2 | δ 9.27 (s, 1H), 9.23 (s, 1H), 8.26 (d, J = 6.6 Hz, 1H), 8.04 (s, 1H), 7.98 (s, 1H), 7.78 (d, J = 8.7 Hz, 2H), 7.64 (d, J = 8.0 Hz, 1H), 7.43 (d, J = 8.8 Hz, 2H), 7.39-7.30 (m, 4H), 7.30-7.24 (m, 2H), 7.22 (d, J = 7.6 Hz, 3H), 7.11 (t, J = 7.0 Hz, 1H), 5.34 (m, 1H), 3.58-3.57 (m, 1H), 3.16 (m, 3H), 2.77 (m, 1H) |
| 66 | 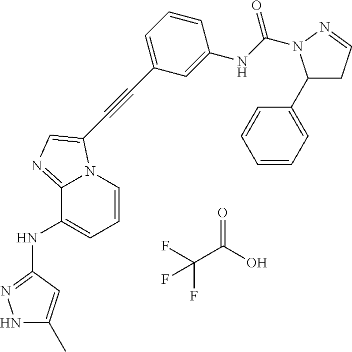 [SMILES: Cc1cc(Nc2cccn3c(C#Cc4cccc(NC(=O)N5N=CCC5c5ccccc5)c4)cnc23)n[nH]1 ; O=C(O)C(F)(F)F] | N-(3-((8-((5- methyl-1H- pyrazol-3- yl)amino)imidazo [1,2-a]pyridin- 3- yl)ethynyl)phenyl)- 5-phenyl-4,5- dihydro-1H- pyrazole-1- carboxamide 2,2,2- trifluoroacetate | 501.2 | δ 9.26 (s, 1H), 8.75 (s, 1H), 8.05- 7.92 (m, 3H), 7.87 (d, J = 7.7 Hz, 1H), 7.64 (d, J = 8.2 Hz, 1H), 7.34 (m, 3H), 7.29- 7.19 (m, 5H), 7.06 (t, J = 7.2 Hz, 1H), 5.94 (br, 1H), 5.34 (m, 1H), 3.60-3.58 (m, 1H), 2.76 (m, 1H), 2.22 (s, 3H) |
| 67 | 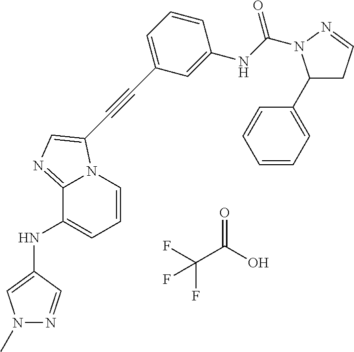 [SMILES: Cn1cc(Nc2cccn3c(C#Cc4cccc(NC(=O)N5N=CCC5c5ccccc5)c4)cnc23)cn1 ; O=C(O)C(F)(F)F] | N-(3-((8-((1- methyl-1H- pyrazol-4- yl)amino)imidazo [1,2-a]pyridin- 3- yl)ethynyl)phenyl)- 5-phenyl-4,5- dihydro-1H- pyrazole-1- carboxamide 2,2,2- trifluoroacetate | 501.2 | δ 9.26 (s, 1H), 8.85 (s, 1H), 8.03- 7.88 (m, 3H), 7.88-7.82 (m, 1H), 7.66-7.61 (m, 1H), 7.54 (d, J = 2.2 Hz, 1H), 7.34 (m, 3H), 7.29- 7.19 (m, 5H), 7.03 (t, J = 7.2 Hz, 1H), 6.12 (d, J = 2.2 Hz, 1H), 5.34 (m, 1H), 3.80 (s, 3H), 3.87 (s, 1H), 2.76 (m, 1H) |
| 68 | 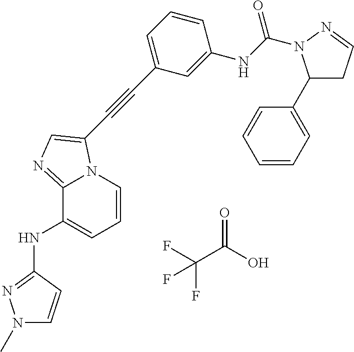 [SMILES: Cn1ccc(Nc2cccn3c(C#Cc4cccc(NC(=O)N5N=CCC5c5ccccc5)c4)cnc23)n1 ; O=C(O)C(F)(F)F] | N-(3-((8-((1- methyl-1H- pyrazol-3- yl)amino)imidazo [1,2-a]pyridin- 3- yl)ethynyl)phenyl)- 5-phenyl-4,5- dihydro-1H- pyrazole-1- carboxamide 2,2,2- trifluoroacetate | 501.2 | δ 9.27 (s, 1H), 8.19 (br, 1H), 8.01 (s, 1H), 7.99- 7.91 (m, 2H), 7.83 (s, 1H), 7.67- 7.60 (m, 1H), 7.52 (s, 1H), 7.39- 7.17 (m, 8H), 6.99 (t, J = 7.2 Hz, 1H), 6.64 (d, J = 7.6 Hz, 1H), 5.34 (m, 1H), 3.84 (s, 3H), 3.57 (m, 1H), 2.76 (m, 1H) |

### EXPERIMENTAL EXAMPLE 1

### Evaluation of Kinase Inhibitory Activity

The following experiment was performed to evaluate the inhibitory activity of the example compounds according to the present invention against various enzymes.

Particularly, among the example compounds of the present invention, the compound of Example 1 was selected, and the enzyme (kinase) selectivity was determined by requesting DiscoverX, and the experiment was conducted using a scanMAX™ Kinase analysis panel.

At this time, the concentration of the drug treated to the enzyme was 1 μM in DMSO, and the control percentage (% control) was determined in the same manner as shown in equation 1 below, and the results are shown in Table 4 below.

(Example Compound−Positive Control)/(Negative Control−Positive Control)×100 [Equation 1]

Herein, the positive control refers to a compound showing the control percentage of 0%, and the negative control refers DMSO showing the control percentage of 100%. In addition, the enzyme selectivity of the present invention was determined to have activity against the enzyme when the control percentage for each enzyme was less than 35% (<35%).

The results are shown in Table 8 below.

TABLE 8

| | Example | | Example | | Example |
|---|---|---|---|---|---|
| | 1 | | 1 | | 1 |
| AAK1 | 99 | FES | 100 | PCTK3 | 69 |
| ABL1 (E255K)- | 30 | FGFR1 | 99 | PDGFRA | 1.8 |
| phosphorylated | | | | | |
| ABL1 (F317I)- | 0 | FGFR2 | 97 | PDGFRB | 0 |
| nonphosphorylated | | | | | |
| ABL1 (F317I) - | 32 | FGFR3 | 91 | PDPK1 | 92 |
| phosphorylated | | | | | |
| ABL1 (F317L)- | 0 | FGFR3 | 83 | PFCDPK1 | 64 |
| nonphosphorylated | | (G697C) | | (P. faleiparum) | |
| ABL1 (F317L)- | 18 | FGFR4 | 100 | PFPK5 | 89 |
| phosphorylated | | | | (P. falciparum) | |
| ABL1 (H396P)- | 0 | FGR | 97 | PFTAIRE2 | 100 |
| nonphosphorylated | | | | | |
| ABL1 (H396P)- | 22 | FLT1 | 98 | PFTK1 | 85 |
| phosphorylated | | | | | |
| ABL1 (M351T)- | 23 | FLT3 | 9.6 | PHKG1 | 100 |
| phosphorylated | | | | | |
| ABL1 (Q252H)- | 0 | FLT3 | 65 | PHK32 | 100 |
| nonphosphorylated | | (D835B) | | | |
| ABL1 (Q252H)- | 50 | FLT3 | 66 | PIK3C2B | 98 |
| phosphorylated | | (D835V) | | | |
| ABL1 (T315I)- | 12 | FLT3 | 74 | PIK3C2G | 74 |
| nonphosphorylated | | (D835Y) | | | |
| ABL1 (T315I)- | 62 | FLT3 | 49 | PIK3CA | 85 |
| phosphorylated | | (ITD) | | | |
| ABL1 (Y253F)- | 100 | FLT3 | 90 | PIK3CA | 90 |
| phosphorylated | | (ITD, D835V) | | (C420R) | |
| ABL1- | 1.1 | FLT3 | 82 | PIK3CA | 85 |
| nonphosphorylated | | (ITD, F691L) | | (E542K) | |
| ABL1- | 21 | FLT3 | 36 | PIK3CA | 92 |
| phosphorylated | | (K663Q) | | (E545A) | |
| ABL2 | 27 | FLT3 | 26 | PIK3CA | 81 |
| | | (N841I) | | (E545K) | |
| ACVR1 | 100 | FLT3 | 76 | PIK3CA | 92 |
| | | (R834Q) | | (H1047L) | |
| ACVR1B | 92 | FLT3- | 81 | PIK3CA | 100 |
| | | autoinhibited | | (H1047Y) | |
| ACVR2A | 100 | FLT4 | 95 | PIK3CA | 75 |
| | | | | (I800L) | |
| ACVR2B | 86 | FRK | 37 | PIK3CA | 95 |
| | | | | (M1043I) | |
| ACVRL1 | 93 | FYN | 90 | PIK3CA | 78 |
| | | | | (Q546K) | |
| ADCK3 | 90 | GAK | 99 | PIK3CB | 49 |
| ADCK4 | 86 | GCN2 | 87 | PIK3CD | 96 |
| | | | | (Kin. Dom. 2, | |
| | | | | S808G) | |
| AKT1 | 93 | GRK1 | 88 | PIK3CG | 98 |
| AKT2 | 95 | GRK2 | 87 | PIK4CB | 92 |
| AKT3 | 96 | GRK3 | 100 | PIKFYVE | 63 |
| ALK | 93 | GRK4 | 66 | PIM1 | 80 |
| ALK | 100 | GRK7 | 96 | PIM2 | 95 |
| (C1156Y) | | | | | |
| ALK | 87 | GSK3A | 100 | PIM3 | 86 |
| (L1196M) | | | | | |
| AMPK- | 91 | GSK3B | 91 | PIP5K1A | 93 |
| alpha1 | | | | | |
| AMPK- | 89 | HASPIN | 96 | PIP5K1C | 79 |
| alpha2 | | | | | |
| ANKK1 | 72 | HCK | 77 | PIP5K2B | 94 |
| ARK5 | 100 | HIPK1 | 95 | PIP5K2C | 95 |
| ASK1 | 80 | HIPK2 | 99 | PKAC- | 92 |
| | | | | alpha | |
| ASK2 | 93 | HIPK3 | 95 | PKAC- | 94 |
| | | | | beta | |
| AURKA | 86 | HIPK4 | 54 | PKMYT1 | 95 |
| AURKB | 72 | HPK1 | 96 | PKN1 | 90 |
| AURKC | 100 | HUNK | 74 | PKN2 | 83 |
| AXL | 82 | ICK | 79 | PKNB | 100 |
| | | | | (M. tuberculosis) | |
| BIKE | 100 | IGF1R | 94 | PLK1 | 96 |
| BLK | 76 | IKK- | 96 | PLK2 | 91 |
| | | alpha | | | |
| BMPR1A | 97 | IKK- | 91 | PLK3 | 88 |
| | | beta | | | |
| BMPR1B | 90 | IKK- | 94 | PLK4 | 100 |
| | | epsilon | | | |
| BMPR2 | 97 | INSR | 94 | PRKCD | 94 |
| BMX | 87 | INSRR | 100 | PRKCE | 80 |
| BRAF | 6 | IRAK1 | 88 | PRKCH | 98 |
| BRAF | 0.75 | IRAK3 | 100 | PRKCI | 100 |
| (V600E) | | | | | |
| BRK | 96 | IRAK4 | 94 | PRKCQ | 76 |
| BRSK1 | 100 | ITK | 90 | PRKD1 | 98 |
| BRSK2 | 98 | JAK1 | 97 | PRKD2 | 100 |
| | | (JH1domain- | | | |
| | | catalytic) | | | |
| BTK | 100 | JAK1 | 90 | PRKD3 | 97 |
| | | (JH2domain- | | | |
| | | pseudokinase) | | | |
| BUB1 | 86 | JAK2 | 76 | PRKG1 | 99 |
| | | (JH1domain- | | | |
| | | catalytic) | | | |
| CAMK1 | 91 | JAK3 | 70 | PRKG2 | 100 |
| | | (JH1domain- | | | |
| | | catalytic) | | | |
| CAMK1B | 88 | JNK1 | 90 | PRKR | 88 |
| CAMK1D | 96 | JNK2 | 97 | PRKX | 100 |
| CAMK1G | 100 | JNX3 | 83 | PRP4 | 60 |
| CAMK2A | 100 | KIT | 0.05 | PYK2 | 95 |
| CAMK2B | 100 | KIT | 29 | QSK | 77 |
| | | (A829P) | | | |
| CAMK2D | 100 | KIT | 59 | RAF1 | 9.1 |
| | | (D816H) | | | |
| CAMK2G | 98 | KIT | 84 | RET | 100 |
| | | (D816V) | | | |
| CAMK4 | 97 | KIT | 0.25 | RET | 96 |
| | | (L576P) | | (M918T) | |
| CAMKK1 | 90 | KIT | 0 | RET | 100 |
| | | (V559D) | | (V804L) | |
| CAMKK2 | 100 | KIT | 87 | RET | 100 |
| | | (V559D, T6701) | | (V804M) | |
| CASK | 94 | KIT | 44 | RIOK1 | 100 |
| | | (V559D, V654A) | | | |
| CDC2L1 | 92 | KIT- | 67 | RIOK2 | 74 |
| | | autoinhibited | | | |
| CDC2L2 | 88 | LATS1 | 99 | RIOK3 | 100 |
| CDC2L5 | 54 | LATS2 | 100 | RIPK1 | 0 |
| CDK11 | 7.9 | LCK | 53 | RIPK2 | 78 |
| CDK2 | 99 | LIMK1 | 88 | RIPK4 | 97 |
| CDK3 | 100 | LIMK2 | 71 | RIPK5 | 72 |
| CDK4 | 95 | LKB1 | 100 | ROCK1 | 87 |
| CDK4- | 83 | LOK | 14 | ROCK2 | 82 |
| cyclinD1 | | | | | |
| CDK4- | 87 | LRRK2 | 66 | ROS1 | 100 |
| cyclinD3 | | | | | |
| CDK5 | 93 | LRRK2 | 90 | RPS6KA4 | 95 |
| | | (G2019S) | | (Kin. Dom. 1-N- | |
| | | | | terminal) | |
| CDK7 | 50 | LTK | 34 | RPS6KA4 | 98 |
| | | | | (Kin. Dom. 2-C- | |
| | | | | terminal) | |
| CDK8 | 0 | LYN | 69 | RPS6KA5 | 100 |
| | | | | (Kin. Dom. 1-N- | |
| | | | | terminal) | |
| CDK9 | 42 | LZK | 100 | RPS6KA5 | 100 |
| | | | | (Kin. Dom. 2-C- | |
| | | | | terminal) | |
| CDKL1 | 95 | MAK | 85 | RSK1 | 90 |
| | | | | (Kin. Dom. 1-N- | |
| | | | | terminal) | |
| CDKL2 | 0 | MAP3K1 | 81 | RSK1 | 99 |
| | | | | (Kin. Dom. 2-C- | |
| | | | | terminal) | |
| CDKL3 | 99 | MAP3K15 | 88 | RSK2 | 91 |
| | | | | (Kin. Dom. 1-N- | |
| | | | | terminal) | |
| CDKL5 | 100 | MAP3K2 | 85 | RSK2 | 91 |
| | | | | (Kin. Dom. 2-C- | |
| | | | | terminal) | |
| CHEK1 | 100 | MAP3K3 | 95 | RSK3 | 75 |
| | | | | (Kin.Dom. 1-N- | |
| | | | | terminal) | |
| CHEK2 | 100 | MAP3K4 | 77 | RSK3 | 93 |
| | | | | (Kin. Dom. 2-C- | |
| | | | | terminal) | |
| CIT | 19 | MAP4K2 | 94 | RSK4 | 75 |
| | | | | (Kin. Dom. 1-N- | |
| | | | | terminal) | |
| CLK1 | 88 | MAP4K3 | 88 | RSK4 | 100 |
| | | | | (Kin. Dom. 2-C- | |
| | | | | terminal) | |
| CLK2 | 99 | MAP4K4 | 83 | S6K1 | 83 |
| CLK3 | 89 | MAP4K5 | 78 | SBK1 | 100 |
| CLK4 | 93 | MAPKAPK2 | 100 | SGK | 90 |
| CSF1R | 6.6 | MAPKAPK5 | 100 | SgK110 | 100 |
| CSF1R- | 83 | MARK1 | 98 | SGK2 | 75 |
| autoinhibited | | | | | |
| CSK | 88 | MARK2 | 100 | SGK3 | 83 |
| CSNK1A1 | 91 | MARK3 | 100 | SIK | 89 |
| CSNK1A1L | 100 | MARK4 | 97 | SIK2 | 93 |
| CSNK1D | 100 | MAST1 | 84 | SLK | 71 |
| CSNK1E | 96 | MEK1 | 95 | SNARK | 98 |
| CSNK1G1 | 98 | MEK2 | 97 | SNRK | 100 |
| CSNK1G2 | 100 | MEK3 | 81 | SRC | 94 |
| CSNK1G3 | 93 | MEK4 | 73 | SRMS | 54 |
| CSNK2A1 | 100 | MEK5 | 53 | SRPK1 | 87 |
| CSNK2A2 | 83 | MEK6 | 94 | SRPK2 | 73 |
| CTK | 92 | MELK | 53 | SRPK3 | 99 |
| DAPK1 | 99 | MERTK | 79 | STK16 | 96 |
| DAPK2 | 100 | MET | 68 | STK33 | 77 |
| DAPK3 | 99 | MET | 94 | STK35 | 100 |
| | | (M1250T) | | | |
| DCAMKL1 | 96 | MET | 60 | STK36 | 38 |
| | | (Y1235D) | | | |
| DCAMKL2 | 83 | MINK | 80 | STK39 | 75 |
| DCAMKL3 | 98 | MKK7 | 88 | SYK | 91 |
| DDR1 | 0.2 | MKNK1 | 92 | TAK1 | 80 |
| DDR2 | 0 | MKNK2 | 61 | TAOK1 | 74 |
| DLK | 91 | MLCK | 97 | TAOK2 | 94 |
| DMPK | 99 | MLK1 | 70 | TAOK3 | 84 |
| DMPK2 | 84 | MLK2 | 68 | TBK1 | 96 |
| DRAK1 | 100 | MLK3 | 98 | TEC | 96 |
| DRAK2 | 96 | MRCKA | 83 | TESK1 | 86 |
| DYRK1A | 70 | MRCKB | 91 | TGFBR1 | 87 |
| DYRK1B | 90 | MST1 | 93 | TGFBR2 | 96 |
| DYRK2 | 84 | MST1R | 89 | TIE1 | 37 |
| EGFR | 100 | MST2 | 97 | TIE2 | 75 |
| EGFR | 100 | MST3 | 95 | TLK1 | 90 |
| (E746- | | | | | |
| A750del) | | | | | |
| EGFR | 100 | MST4 | 98 | TLK2 | 94 |
| (G719C) | | | | | |
| EGFR | 100 | MTOR | 96 | TNIK | 82 |
| (G719S) | | | | | |
| EGFR | 70 | MUSK | 24 | TNK1 | 96 |
| (L747- | | | | | |
| E749del, | | | | | |
| A750P) | | | | | |
| EGFR(L747- | 100 | MYLK | 91 | TNK2 | 99 |
| S752del, | | | | | |
| P753S) | | | | | |
| EGFR | 100 | MYLK2 | 94 | TNNI3K | 77 |
| (L747- | | | | | |
| T751del, Sins) | | | | | |
| EGFR | 72 | MYLK4 | 86 | TRKA | 62 |
| (L858R) | | | | | |
| EGFR | 96 | MYO3A | 90 | TRKB | 83 |
| (L858R, | | | | | |
| T790M) | | | | | |
| EGFR | 100 | MYO3B | 92 | TRKC | 83 |
| (L861Q) | | | | | |
| EGFR | 100 | NDR1 | 97 | TRPM6 | 89 |
| (S752- | | | | | |
| I759del) | | | | | |
| EGFR | 96 | NDR2 | 86 | TSSK1B | 100 |
| (T790M) | | | | | |
| EIF2AK1 | 100 | NEK1 | 98 | TSSK3 | 97 |
| EPHA1 | 87 | NEK10 | 84 | TTK | 66 |
| EPHA2 | 86 | NEK11 | 98 | TXK | 67 |
| EPH13 | 59 | NEK2 | 100 | TYK2 | 66 |
| | | | | (JH1domain- | |
| | | | | catalytic) | |
| EPHA4 | 97 | NEK3 | 76 | TYK2 | 100 |
| | | | | (JH2domain- | |
| | | | | pseudokinase) | |
| EPHA5 | 96 | NEK4 | 95 | TYRO3 | 95 |
| EPHA6 | 95 | NEK5 | 97 | ULK1 | 84 |
| EPHA7 | 91 | NEK6 | 95 | ULK2 | 93 |
| EPHA8 | 84 | NEK7 | 100 | ULK3 | 71 |
| EPHB1 | 82 | NEK9 | 100 | VEGFR2 | 70 |
| EPHB2 | 92 | NIK | 82 | VPS34 | 65 |
| EPHB3 | 93 | NTM1 | 99 | VRK2 | 98 |
| EP9B4 | 100 | NLK | 85 | WEE1 | 92 |
| EPHB6 | 50 | OSR1 | 100 | WEE2 | 100 |
| ERBB2 | 54 | p38- | 86 | WNK1 | 97 |
| | | alpha | | | |
| ERBB3 | 98 | p38- | 94 | WNK2 | 98 |
| | | beta | | | |
| ERBB4 | 87 | p38- | 88 | WNK3 | 96 |
| | | delta | | | |
| ERK1 | 94 | p38- | 97 | WNK4 | 45 |
| | | gamma | | | |
| ERK2 | 94 | PAK1 | 97 | YANK1 | 100 |
| ERK3 | 92 | PAK2 | 75 | YANK2 | 100 |
| ERK4 | 96 | PAK3 | 30 | YANK3 | 91 |
| ERK5 | 100 | PAK4 | 100 | YES | 100 |
| ERK8 | 87 | PAK6 | 96 | YSK1 | 71 |
| ERN1 | 89 | PAK7 | 95 | YSK4 | 65 |
| FAK | 94 | PCTK1 | 89 | ZAK | 63 |
| FER | 100 | PCTK2 | 80 | ZAP70 | 100 |

As shown in Table 8, the example compounds according to the present invention exhibited the percentage control values of less than 35% for ABL1(E255K), ABL1(F317T), ABL1(F317L), ABL1(H396P), ABL1(M351T), ABL1(Q252H), ABL1(T315I), ABL1, ABL2, BRAF, BRAF(V600E), CDK11, CDK8, CDKL2, CIT, CSF1R, DDR1, DDR2, EPHB6, FLT3, FLT3(D835H), FLT3(ITD), FLT3(K663Q), FLT3(N841I), HIPK4, KIT, KIT(A829P), KIT(L576P), KIT(V559D), KIT(V559D,T670I), LOK, LTK, MEK5, MKNK2, MET(Y1235D), MUSK, PAK3, PDGFRA, PDGFRB, RAF1, RIPK1, ROCK1, TIE1 and VEGFR2 kinases. These results indicated that the example compounds according to the present invention have inhibitory activity against the enzymes listed above, so that it can be seen that the compounds above may have a useful effect when used in diseases related to the enzymes listed above.

Therefore, the compounds according to the present invention can be effectively used as a composition for the treatment or prevention of ABL1, ABL2, BRAF, CDK11, CDK8, CDKL2, CIT, CSF1R, DDR1, DDR2, FLT3, KIT, LOK, LTK, MUSK, PAK3, PDGFRA, PDGFRB, RAF1 and RIPK1-related diseases.

### EXPERIMENTAL EXAMPLE 2

### Evaluation of RIPK1 (Receptor-Interacting Serine/Threonine Protein Kinase 1) Inhibitory Activity

The following experiment was performed to evaluate the inhibitory activity of the example compounds according to the present invention against RIPK1 (receptor-interacting serine/threonine protein kinase 1).

The example compound was reacted with the purified human GST-RIPK1 (1375, signalchem) enzyme to evaluate the enzyme inhibitory activity in the following manner. The composition of the reaction buffer used herein was as follows: 40 mM Tris-Hcl pH 7.4, 20 mM MgCl₂, 0.5 mg/ml, BSA and 0.5 uM DTT. All the test materials were reacted in the reaction buffer. After reacting human GST-RIPK1 (1375, 10 ng), purified ATP (50 uM) and a specific substrate solution at 25° C. for 4 hours, the enzyme activity was confirmed using an in vitro ADP-Glo™ kinase assay (promega). Luminoscence was measured by reacting the enzyme activity reaction solution, ADP-Glo reaction solution, and enzyme activity detection solution at the ratio of 2:2:1. The degree of enzyme activity inhibition according to the treatment concentration of each compound was calculated based on the fluorescence of the enzyme activity of the solvent control not treated with the compound. At this time, the concentration of each compound inhibiting 50% of the enzyme activity was determined as IC₅₀ (nM). IC₅₀ of each compound was determined with three data sets and calculated using Prism (version 7.01, GraphPad) software.

The results are shown in Table 9 below.

### EXPERIMENTAL EXAMPLE 3

### Evaluation of Cell Protection Effect Under Apoptosis-Inducing Condition

The cell protection effect of the compounds according to the present invention under the condition of inducing apoptosis by TNF-α was confirmed by MTS assay. An apoptosis inducing factor such as TNF-α was treated to induce apoptosis of human Jurkat T cells deficient in FADD, to which the example compound was treated, and the cell protection effect was confirmed through the following analysis. Jurkat T cells deficient in FADD were cultured using RPMI medium (Hyclone) containing 10% FBS. When performing the test, the cells were seeded in a 96-well plate containing a medium suitable for the cell line at the density of 10,000 cells/well, and cultured in a 37° C. 5% CO₂ incubator for 24 hours. Then, each well was treated with 40 ng of TNF-α, and the compounds prepared in the above examples were each treated with a 3-fold concentration gradient with 1 μM as the highest concentration. As the solvent control, dimethyl sulfoxide (DMSO) was treated at the concentration of 0.05% (v/v), which was the same concentration as that used for the compound treatment. Then, the cells were cultured for 50 hours. To confirm the degree of viability of the cells, a mixture provided in CellTiter-Gloe Luminescent Cell Viability Assay Kit (Promega) was added to the culture medium of the cells, followed by further culture at 37° C. for 30 minutes. Then, luminescence fluorescence was measured. The degree of inhibition of apoptosis induction according to the treatment concentration of each compound was calculated based on the fluorescence of the solvent control cells not treated with the compound. At this time, the concentration of each compound inhibiting 50% was determined as EC₅₀ (μM). EC₅₀ of each compound was calculated using Prism (version 7.01, GraphPad) software.

TABLE 9

| | Enzyme | I2.I Cell |
|---|---|---|
| Example | IC₅₀ (nM) | EC₅₀ (nM) |
| 1 | 30.9 | 0.17 |
| 2 | 5.1 | 0.06 |
| 3 | 221 | 314 |
| 4 | 7.68 | 1.7 |
| 5 | 4.56 | 0.4 |
| 6 | >1,000 | 249 |
| 7 | 134 | 1.9 |
| 8 | 83.6 | 0.59 |
| 9 | 19.5 | 0.41 |
| 10 | 801 | 326 |
| 11 | 47.4 | 0.35 |
| 12 | 19.1 | 0.2 |
| 13 | 503 | 321 |
| 14 | 5.3 | 0.2 |
| 15 | 4.3 | 0.1 |
| 16 | 1.8 | 0.05 |
| 17 | 1.8 | 0.2 |
| 18 | 2.4 | 0.3 |
| 19 | 3.8 | 0.26 |
| 20 | 0.52 | 0.021 |
| 21 | >1,000 | 588 |
| 22 | >1,000 | >1,000 |
| 23 | 32.2 | 1.2 |
| 24 | 2.76 | 0.44 |
| 25 | 878 | 364 |
| 26 | 6.9 | 201 |
| 27 | 2.35 | 55.4 |
| 28 | 10.1 | 0.8 |
| 29 | 5.83 | 0.3 |
| 30 | 399 | 321 |
| 31 | 10.2 | 1.43 |
| 32 | 4.83 | 0.63 |
| 33 | >1,000 | 32 |
| 34 | 18.9 | 43 |
| 35 | 61.6 | 3.87 |
| 36 | 2.98 | 12 |
| 37 | 1.22 | 1.84 |
| 38 | 1.1 | 106 |
| 39 | 4.9 | 2.8 |
| 40 | 19 | 12.4 |
| 41 | 33 | 64.6 |
| 42 | 12.7 | 1.5 |
| 43 | 277 | 6.7 |
| 44 | 18.1 | 0.37 |
| 45 | 8.8 | 0.09 |
| 46 | 205 | 97.7 |
| 47 | 50.1 | >1,000 |
| 48 | 3.4 | 1.4 |
| 49 | 1.9 | 0.3 |
| 50 | >1,000 | 197 |
| 51 | 1.22 | 1.84 |
| 52 | 775 | >1,000 |
| 53 | 731 | 975 |
| 54 | 32.6 | >1,000 |
| 55 | 13.4 | 1.6 |
| 56 | 30.5 | 0.3 |
| 57 | 23.8 | 0.3 |
| 58 | 15 | 3.6 |
| 59 | 127 | 17.6 |
| 60 | 64.1 | 0.4 |
| 61 | 96.7 | 0.4 |
| 62 | 179 | 1.7 |
| 63 | 39.6 | 0.9 |
| 64 | 119 | 1.1 |
| 65 | 51.4 | 0.2 |
| 66 | 30.6 | 0.22 |
| 67 | 19 | 0.08 |
| 68 | 38.6 | 0.12 |

Table 9 shows the results of measuring the RIPK1 inhibitory activity and the cell protection effect of each experimental compound on FADD-deficient Jurkat T cells under the apoptosis-inducing condition.

As shown in Table 9, it was confirmed that the example compounds according to the present invention exhibit excellent enzyme inhibitory activity and cell protection effect. Therefore, it can be seen that the compounds according to the present invention have excellent cell protection activity under the apoptosis-inducing condition, as confirmed in the above experiment.

### INDUSTRIAL APPLICABILITY

The N-(3-(imidazo[1,2-b]pyridazin-3-ylethynyl)-4-methylphenyl)-5-phenyl-4,5-dihydro-1H-pyrazole-1-carboxamide derivative provided in one aspect of the present invention exhibits excellent inhibitory activity against at least one kinase selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK, and thus being useable as a therapeutic agent for kinase-related disease.

1. A compound represented by formula 1, a stereoisomer thereof, a hydrate thereof, or a pharmaceutically acceptable salt thereof: 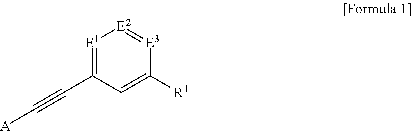
wherein,
E¹ is ═CA¹- or ═N—,
A¹ is —H, C₁₋₁₀ straight or branched alkyl or halogen;
E² is ═CA²- or ═N—,
A² is —H, C₁₋₁₀ straight or branched alkyl or halogen;
E³ is ═CA³- or ═N—,
A³ is —H, halogen or 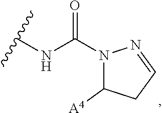
wherein, A⁴ is nonsubstituted or substituted C₆₋₁₀ aryl, or nonsubstituted or substituted 5-10 membered heteroaryl containing one or more heteroatoms selected from the group consisting of N, O and S,
wherein, the substituted C₆₋₁₀ aryl or the substituted 5-10 membered heteroaryl is C₆₋₁₀ aryl or 5-10 membered heteroaryl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen;
R¹ is —H, 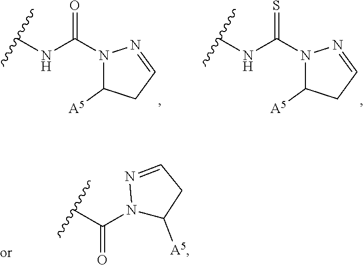
wherein, A⁵ is nonsubstituted, substituted or fused C₆₋₁₀ aryl, nonsubstituted, substituted or fused 5-10 membered heteroaryl containing one or more heteroatoms selected from the group consisting of N, O and S, or nonsubstituted or substituted C₄₋₁₀ cycloalkyl,
wherein, the substituted C₆₋₁₀ aryl, the substituted 5-10 membered heteroaryl, or the substituted C₄₋₁₀ cycloalkyl is C₆₋₁₀ aryl, 5-10 membered heteroaryl, or C₄₋₁₀ cycloalkyl substituted with one or more substituents selected from the group consisting of C₁₋₁₀ straight or branched alkyl nonsubstituted or substituted with one or more halogens, C₁₋₁₀ straight or branched alkoxy nonsubstituted or substituted with one or more halogens, and halogen,
wherein, the fused C₆₋₁₀ aryl or the fused 5-10 membered heteroaryl is C₆₋₁₀ aryl or 5-10 membered heteroaryl fused with 5-6 membered heterocycloalkyl containing one or more Os or phenyl; and
A is 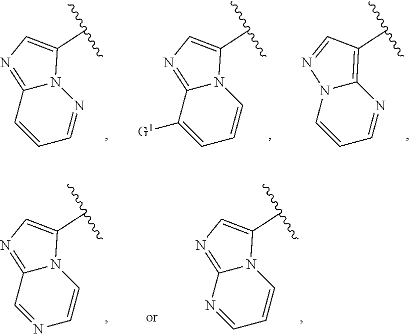
wherein, G¹ is hydrogen, halogen, hydroxy, nitro, C₁₋₁₀ straight or branched alkyl, C₁₋₁₀ straight or branched alkoxy or —NR⁴R⁵,
wherein, R⁴ and R⁵ are independently hydrogen, C₁₋₁₀ straight or branched alkylcarbonyl, C₁₋₁₀ straight or branched alkylaminocarbonyl, C₃₋₆ cycloalkylcarbonyl, C₁₋₁₀ straight or branched alkoxycarbonyl, nonsubstituted or substituted C₆₋₁₀ aryl, or nonsubstituted or substituted 5-10 membered heteroaryl containing one or more heteroatoms selected from the group consisting of N, O and S,
wherein, the substituted C₆₋₁₀ aryl is C₆₋₁₀ aryl substituted with C₁₋₁₀ straight or branched alkylsulfonyl, and
wherein, the substituted 5-10 membered heteroaryl is 5-10 membered heteroaryl substituted with C₁₋₁₀ straight or branched alkyl.

2. The compound, the stereoisomer thereof, the hydrate thereof, or the pharmaceutically acceptable salt thereof according to claim 1, wherein:
E¹ is ═CA¹- or ═N—,
A¹ is —H, C₁₋₅ straight or branched alkyl, or halogen;
E² is ═CA²- or ═N—,
A² is —H, C₁₋₅ straight or branched alkyl, or halogen;
E³ is ═CA³- or ═N—,
A³ is —H, halogen, or 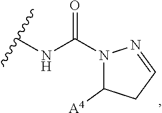
wherein, A⁴ is nonsubstituted or substituted C₆₋₁₀ aryl, or nonsubstituted or substituted 5-10 membered heteroaryl containing one or more heteroatoms selected from the group consisting of N, O and S,
wherein, the substituted C₆₋₁₀ aryl or the substituted 5-10 membered heteroaryl is C₆₋₁₀ aryl or 5-10 membered heteroaryl substituted with one or more substituents selected from the group consisting of C₁₋₅ straight or branched alkyl nonsubstituted or substituted with one or more halogens, C₁₋₅ straight or branched alkoxy nonsubstituted or substituted with one or more halogens, and halogen;
R¹ is —H, 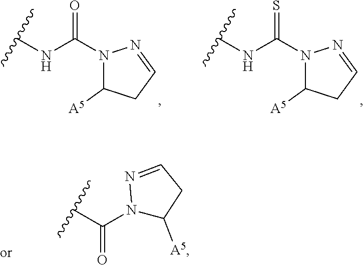
wherein, A⁵ is nonsubstituted, substituted or fused C₆₋₁₀ aryl, nonsubstituted, substituted or fused 5-10 membered heteroaryl containing one or more heteroatoms selected from the group consisting of N, O and S, or nonsubstituted or substituted C₄₋₁₀ cycloalkyl,
wherein, the substituted C₆₋₁₀ aryl, the substituted 5-10 membered heteroaryl, or the substituted C₄₋₁₀ cycloalkyl is C₆₋₁₀ aryl, 5-10 membered heteroaryl, or C₄₋₁₀ cycloalkyl substituted with one or more substituents selected from the group consisting of C₁₋₅ straight or branched alkyl nonsubstituted or substituted with one or more halogens, C₁₋₅ straight or branched alkoxy nonsubstituted or substituted with one or more halogens, and halogen,
wherein, the fused C₆₋₁₀ aryl or the fused 5-10 membered heteroaryl is C₆₋₁₀ aryl or 5-10 membered heteroaryl fused with 5 membered heterocycloalkyl containing two Os or phenyl; and
A is 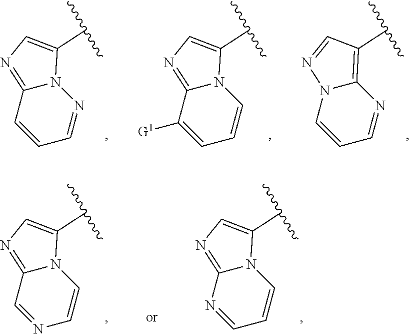
wherein, G¹ is hydrogen, halogen, hydroxy, nitro, C₁₋₅ straight or branched alkyl, C₁₋₅ straight or branched alkoxy or —NR⁴R⁵,
wherein, R⁴ and R⁵ are independently hydrogen, C₁₋₅ straight or branched alkylcarbonyl, C₁₋₅ straight or branched alkylaminocarbonyl, C₃₋₆ cycloalkylcarbonyl, C₁₋₅ straight or branched alkoxycarbonyl, nonsubstituted or substituted C₆₋₁₀ aryl, or nonsubstituted or substituted 5-10 membered heteroaryl containing one or more heteroatoms selected from the group consisting of N, O and S,
wherein, the substituted C₆₋₁₀ aryl is C₆₋₁₀ aryl substituted with C₁₋₅ straight or branched alkylsulfonyl,
wherein, the substituted 5-10 membered heteroaryl is 5-10 membered heteroaryl substituted with C₁₋₅ straight or branched alkyl.

3. The compound, the stereoisomer thereof, the hydrate thereof, or the pharmaceutically acceptable salt thereof according to claim 1, wherein:
E¹ is ═CA¹- or ═N—, wherein A¹ is —H, —H₃, or —F;
E² is ═CA²- or ═N—, wherein A² is —H;
E³ is ═CA³- or ═N—, wherein A³ is —H, —F, or 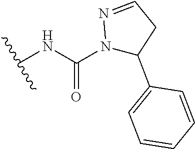 [SMILES: *NC(=O)N1N=CCC1c1ccccc1]
R¹ is —H, 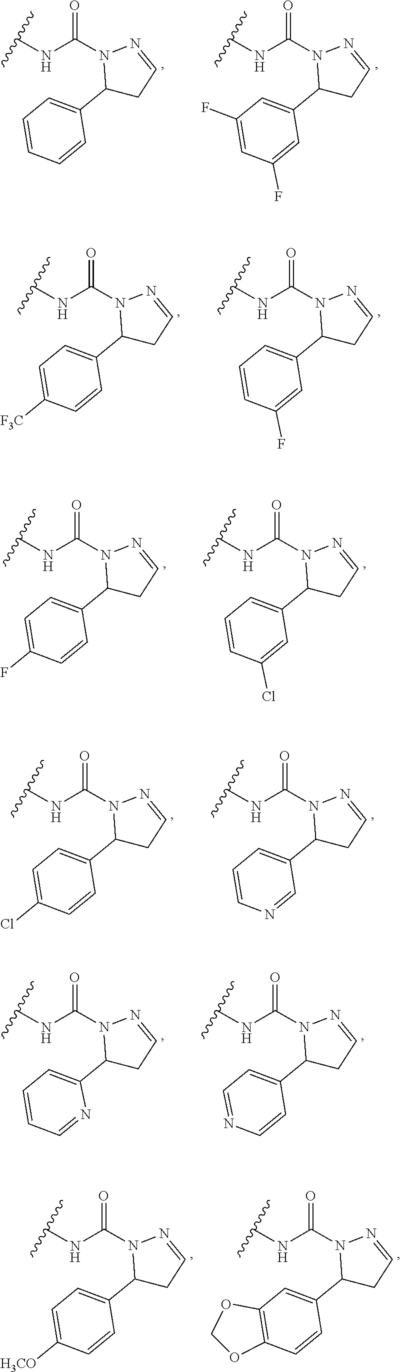 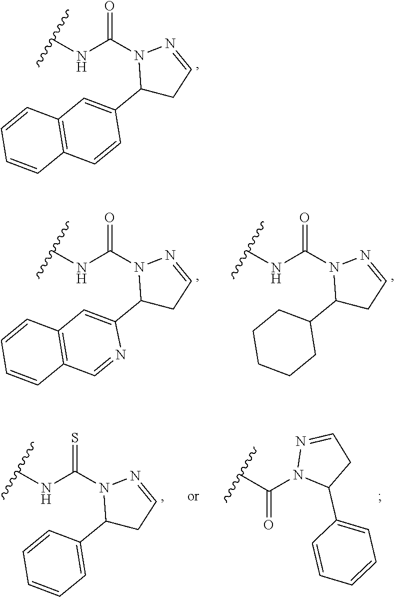 [SMILES: *NC(=O)N1N=CCC1c1ccc2ccccc2c1 ; *NC(=O)N1N=CCC1c1cc2ccccc2cn1 ; *NC(=O)N1N=CCC1C1CCCCC1 ; *NC(=S)N1N=CCC1c1ccccc1 ; *C(=O)N1N=CCC1c1ccccc1]
and
A is

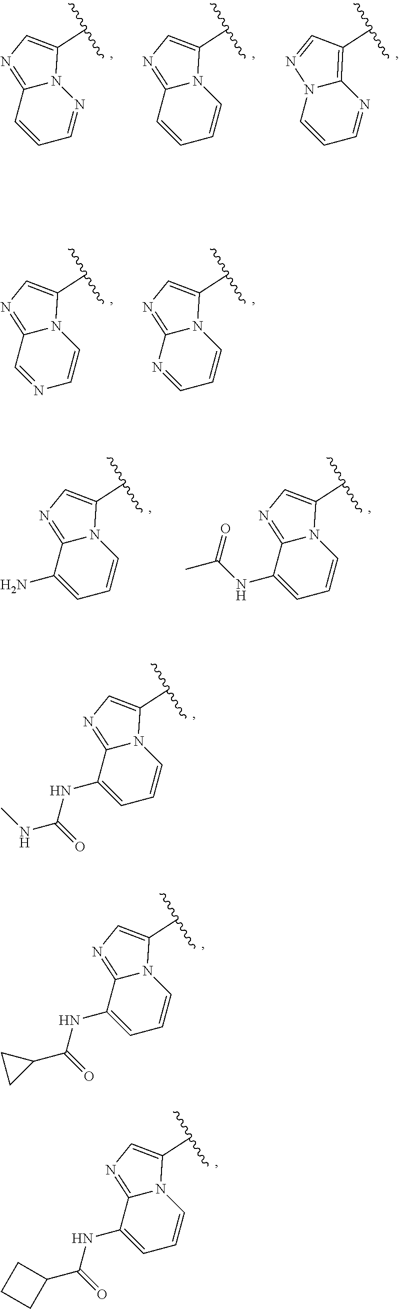

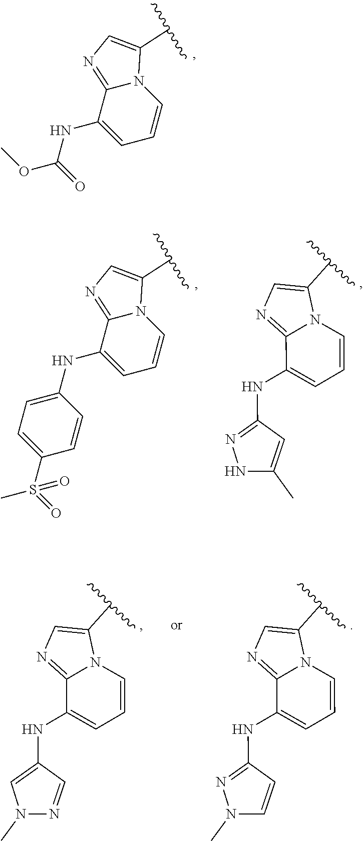 [SMILES: *c1cnc2c(NC(=O)OC)cccn12 ; *c1cnc2c(Nc3ccc(S(C)(=O)=O)cc3)cccn12 ; *c1cnc2c(Nc3cc(C)[nH]n3)cccn12 ; *c1cnc2c(Nc3cnn(C)c3)cccn12 ; *c1cnc2c(Nc3ccn(C)n3)cccn12]

4. The compound, the stereoisomer thereof, the hydrate thereof, or the pharmaceutically acceptable salt thereof according to claim 1, wherein the compound represented by formula 1, the stereoisomer thereof, the hydrate thereof, or the pharmaceutically acceptable salt thereof is selected from the group consisting of the following compounds:
(1) N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;
(2) (S)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;
(3) (R)—N-(3-(imidazo[1,2-b]pyridazin-3-ylethynyl)-4-methylphenyl)-5-phenyl-4,5-dihydro-1H-pyrazole-1-carboxamide 2,2,2-trifluoroacetate;
(4) 5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(5) (S)-5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(6) (R)-5-(3,5-difluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(7) N-(3-(imidazo[1,2-b]pyridazin-3-ylethynyl)-4-methylphenyl)-5-(4-(trifluoromethyl)phenyl)-4,5-dihydro-1H-pyrazole-1-carboxamide 2,2,2-trifluoroacetate;
(8) 5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(9) (S)-5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(10) (R)-5-(3-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(11) 5-(4-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(12) (S)-5-(4-fluorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(13) (R)-5-(4-fluorophenyl)-N-(3-(imidazo[1,2-b]pyridazin-3-ylethynyl)-4-methylphenyl)-4,5-dihydre-1H-pyrazole-1-carboxamide 2,2,2-trifluoroacetate;
(14) 5-(3-chlorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(15) 5-(4-chlorophenyl)-N-(3-(imidazo[1,2-b]pyridazin-3-ylethynyl)-4-methylphenyl)-4,5-dihydro-1H-pyrazole-1-carboxamide 2,2,2-trifluoroacetate;
(16) N-(3-(imidazo[1,2-b]pyridazin-3-ylethynyl)-4-methylphenyl)-5-(pyridin-3-yl)-4,5-dihydro-1H-pyrazole-1-carboxamide 2,2,2-trifluoroacetate;
(17) N-(3-(imidazo[1,2-b]pyridazin-3-ylethynyl)-4-methylphenyl)-5-(pyridin-2-yl)-4,5-dihydro-1H-pyrazole-1-carboxamide 2,2,2-trifluoroacetate;
(18) N-(3-(imidazo[1,2-b]pyridazin-3-ylethynyl)-4-methylphenyl)-5-(pyridin-4-yl)-4,5-dihydro-1H-pyrazole-1-carboxamide 2,2,2-trifluoroacetate;
(19) N-(3-(imidazo[1,2-b]pyridazin-3-ylethynyl)phenyl)-5-phenyl-4,5-dihydro-1H-pyrazole-1-carboxamide 2,2,2-trifluoroacetate;
(20) (S)—N-(3-(imidazo[1,2-b]pyridazin-3-ylethynyl)phenyl)-5-phenyl-4,5-dihydro-1H-pyrazole-1-carboxamide hydrochloride;
(21) (R)—N-(3-(imidazo[1,2-b]pyridazin-3-ylethynyl)phenyl)-5-phenyl-4,5-dihydro-1H-pyrazole-1-carboxamide hydrochloride;
(22) N-(4-(imidazo[1,2-b]pyridazin-3-ylethynyl)phenyl)-5-phenyl-4,5-dihydro-1H-pyrazole-1-carboxamide 2,2,2-trifluoroacetate;
(23) 5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;
(24) (S)-5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;
(25) (R)-5-(3,5-difluorophenyl)-N-(3-(imidazo[1,2-b]pyridazin-3-ylethynyl)phenyl)-4,5-dihydro-1H-pyrazole-1-carboxamide 2,2,2-trifluoroacetate;
(26) N-(3-(imidazo[1,2-b]pyridazin-3-ylethynyl)phenyl)-5-(4-(trifluoromethyl)phenyl)-4,5-dihydro-1H-pyrazole-1-carboxamide 2,2,2-trifluoroacetate;
(27) N-(3-(imidazo[1,2-b]pyridazin-3-ylethynyl)phenyl)-5-(4-methoxyphenyl)-4,5-dihydro-1H-pyrazole-1-carboxamide 2,2,2-trifluoroacetate;
(28) 5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(29) (S)-5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(30) (R)-5-(3-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(31) 5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(32) (S)-5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(33) (R)-5-(4-fluorophenyl)-N-(3-(imidazo[1,2-b]pyridazin-3-ylethynyl)phenyl)-4,5-dihydro-1H-pyrazole-1-carboxamide 2,2,2-trifluoroacetate;
(34) 5-(3-chlorophenyl)-N-(3-(imidazo[1,2-b]pyridazin-3-ylethynyl)phenyl)-4,5-dihydro-1H-pyrazole-1-carboxamide 2,2,2-trifluoroacetate;
(35) 5-(4-chlorophenyl)-N-(3-(imidazo[1,2-b]pyridazin-3-ylethynyl)phenyl)-4,5-dihydro-1H-pyrazole-1-carboxamide 2,2,2-trifluoroacetate;
(36) N-(3-(imidazo[1,2-b]pyridazin-3-ylethynyl)phenyl)-5-(pyridin-3-yl)-4,5-dihydro-1H-pyrazole-1-carboxamide 2,2,2-trifluoroacetate;
(37) N-(3-(imidazo[1,2-b]pyridazin-3-ylethynyl)phenyl)-5-(pyridin-2-yl)-4,5-dihydro-1H-pyrazole-1-carboxamide 2,2,2-trifluoroacetate;
(38) N-(3-(imidazo[1,2-b]pyridazin-3-ylethynyl)phenyl)-5-(pyridin-4-yl)-4,5-dihydro-1H-pyrazole-1-carboxamide hydrochloride;
(39) 5-(benzo[d][1,3]dioxol-5-yl)-N-(3-(imidazo[1,2-b]pyridazin-3-ylethynyl)phenyl)-4,5-dihydro-1H-pyrazole-1-carboxamide 2,2,2-trifluoroacetate;
(40) N-(3-(imidazo[1,2-b]pyridazin-3-ylethynyl)phenyl)-5-(naphthalen-2-yl)-4,5-dihydro-1H-pyrazole-1-carboxamide;
(41) N-(3-(imidazo[1,2-b]pyridazin-3-ylethynyl)phenyl)-5-(isoquinolin-3-yl)-4,5-dihydro-1H-pyrazole-1-carboxamide;
(42) 5-cyclohexyl-N-(3-(imidazo[1,2-b]pyridazin-3-ylethynyl)phenyl)-4,5-dihydro-1H-pyrazole-1-carboxamide;
(43) N-(3-(imidazo[1,2-b]pyridazin-3-ylethynyl)phenyl)-5-phenyl-4,5-dihydro-1H-pyrazole-1-carbothioamide;
(44) N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;
(45) (S)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;
(46) (R)—N-(4-fluoro-3-(imidazo[1,2-b]pyridazin-3-ylethynyl)phenyl)-5-phenyl-4,5-dihydro-1H-pyrazole-1-carboxamide 2,2,2-trifluoroacetate;
(47) N-(2-fluoro-5-(imidazo[1,2-b]pyridazin-3-ylethynyl)phenyl)-5-phenyl-4,5-dihydro-1H-pyrazole-1-carboxamide 2,2,2-trifluoroacetate;
(48) N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;
(49) (S)—N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;
(50) (R)—N-(2-(imidazo[1,2-b]pyridazin-3-ylethynyl)pyridin-4-yl)-5-phenyl-4,5-dihydro-1H-pyrazole-1-carboxamide 2,2,2-trifluoroacetate;
(51) N-(5-(imidazo[1,2-b]pyridazin-3-ylethynyl)pyridin-3-yl)-5-phenyl-4,5-dihydro-1H-pyrazole-1-carboxamide 2,2,2-trifluoroacetate;
(52) N-(4-(imidazo[1,2-b]pyridazin-3-ylethynyl)pyridin-2-yl)-5-phenyl-4,5-dihydro-1H-pyrazole-1-carboxamide 2,2,2-trifluoroacetate;
(53) (3-(imidazo[1,2-b]pyridazin-3-ylethynyl)phenyl) (5-phenyl-4,5-dihydro-1H-pyrazol-1-yl)methanone 2,2,2-trifluoroacetate;
(54) (3-(imidazo[1,2-b]pyridazin-3-ylethynyl)-4-methylphenyl)(5-phenyl-4,5-dihydro-1H-pyrazol-1-yl)methanone 2,2,2-trifluoroacetate;
(55) N-(3-(imidazo[1,2-a]pyridin-3-ylethynyl)phenyl)-5-phenyl-4,5-dihydro-1H-pyrazole-1-carboxamide;
(56) 5-phenyl-N-(3-(pyrazolo[1,5-a]pyrimidin-3-ylethynyl)phenyl)-4,5-dihydro-1H-pyrazole-1-carboxamide;
(57) N-(3-(imidazo[1,2-a]pyrazin-3-ylethynyl)phenyl)-5-phenyl-4,5-dihydro-1H-pyrazole-1-carboxamide;
(58) N-(3-(imidazo[1,2-a]pyrimidin-3-ylethynyl)phenyl)-5-phenyl-4,5-dihydro-1H-pyrazole-1-carboxamide;
(59) N-(3-((8-aminoimidazo[1,2-a]pyridin-3-yl)ethynyl)phenyl)-5-phenyl-4,5-dihydro-1H-pyrazole-1-carboxamide hydrochloride;
(60) N-(3-((8-acetamidoimidazo[1,2-a]pyridin-3-yl)ethynyl)phenyl)-5-phenyl-4,5-dihydro-1H-pyrazole-1-carboxamide;
(61) N-(3-((8-(3-methylureido)imidazo[1,2-a]pyridin-3-yl)ethynyl)phenyl)-5-phenyl-4,5-dihydro-1H-pyrazole-1-carboxamide 2,2,2-trifluoroacetate;
(62) N-(3-((8-(cyclopropanecarboxamido)imidazo[1,2-a]pyridin-3-yl)ethynyl)phenyl)-5-phenyl-4,5-dihydro-1H-pyrazole-1-carboxamide 2,2,2-trifluoroacetate;
(63) N-(3-((8-(cyclobutanecarboxamido)imidazo[1,2-a]pyridin-3-yl)ethynyl)phenyl)-5-phenyl-4,5-dihydro-1H-pyrazole-1-carboxamide 2,2,2-trifluoroacetate;
(64) methyl (3-((3-(5-phenyl-4,5-dihydro-1H-pyrazole-1-carboxamido)phenyl)ethynyl)imidazo[1,2-a]pyridin-8-yl)carbamate;
(65) N-(3-((8-((4-(methylsulfonyl)phenyl)amino)imidazo[1,2-a]pyridin-3-yl)ethynyl)phenyl)-5-phenyl-4,5-dihydro-1H-pyrazole-1-carboxamide 2,2,2-trifluoroacetate;
(66) N-(3-((8-((5-methyl-1H-pyrazol-3-yl)amino)imidazo[1,2-a]pyridin-3-yl)ethynyl)phenyl)-5-phenyl-4,5-dihydro-1H-pyrazole-1-carboxamide 2,2,2-trifluoroacetate;
(67) N-(3-((8-((1-methyl-1H-pyrazol-4-yl)amino)imidazo[1,2-a]pyridin-3-yl)ethynyl)phenyl)-5-phenyl-4,5-dihydro-1H-pyrazole-1-carboxamide 2,2,2-trifluoroacetate; and
(68) N-(3-((8-((1-methyl-1H-pyrazol-3-yl)amino)imidazo[1,2-a]pyridin-3-yl)ethynyl)phenyl)-5-phenyl-4,5-dihydro-1H-pyrazole-1-carboxamide 2,2,2-trifluoroacetate.

5. A pharmaceutical composition comprising the compound represented by formula 1, the stereoisomer thereof, the hydrate thereof, or the pharmaceutically acceptable salt thereof of claim 1 as an active ingredient for the prevention or treatment of kinase-related disease.

6. The pharmaceutical composition according to claim 5, wherein the kinase is one or more kinases selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK.

7. The pharmaceutical composition according to claim 5, wherein the kinase-related disease is a disease selected from the group consisting of inflammatory bowel disease, Crohn's disease, ulcerative colitis, psoriasis, retinal detachment, retinitis pigmentosa, macular degeneration, pancreatitis, atopic dermatitis, rheumatoid arthritis, spondylitis, gout, Sjogren's syndrome, systemic scleroderma, anti-phospholipid syndrome, vasculitis, osteoarthritis, non-alcoholic steatohepatitis, alcoholic steatohepatitis, primary sclerosing cholangitis, nephritis, celiac disease, transplant rejection, sepsis, systemic inflammatory reaction syndrome, myocardial infarction, Huntington's disease, Alzheimer's disease, Parkinson's disease, allergic disease, asthma, atopic dermatitis, multiple sclerosis, type I diabetes, Wegener's granulomatosis, pulmonary sarcoidosis, Behcet's disease, motor neuron disease, chronic obstructive pulmonary disease, cancer and periodontitis.

8. A health functional food composition comprising the compound represented by formula 1, the stereoisomer thereof, the hydrate thereof, or the pharmaceutically acceptable salt thereof of claim 1 as an active ingredient for the prevention or amelioration of kinase-related disease.

9. The health functional food composition according to claim 8, wherein the kinase is one or more kinases selected from the group consisting of ABL1, ABL2, AURKB, BRK, CDK11, CDK8, CDK9, CDKL2, CIT, DDR1, FLT3, HIPK4, HUNK, JAK3, KIT, LOK, LTK, MET, MLK2, MUSK, MYO3A, PAK3, PCTK3, PDGFRA, PDGFRB, RIPK1, TIE1 and ZAK.

10. The health functional food composition according to claim 8, wherein the kinase-related disease is a disease selected from the group consisting of inflammatory bowel disease, Crohn's disease, ulcerative colitis, psoriasis, retinal detachment, retinitis pigmentosa, macular degeneration, pancreatitis, atopic dermatitis, rheumatoid arthritis, spondylitis, gout, Sjogren's syndrome, systemic scleroderma, anti-phospholipid syndrome, vasculitis, osteoarthritis, non-alcoholic steatohepatitis, alcoholic steatohepatitis, primary sclerosing cholangitis, nephritis, celiac disease, transplant rejection, sepsis, systemic inflammatory reaction syndrome, myocardial infarction, Huntington's disease, Alzheimer's disease, Parkinson's disease, allergic disease, asthma, atopic dermatitis, multiple sclerosis, type I diabetes, Wegener's granulomatosis, pulmonary sarcoidosis, Behcet's disease, motor neuron disease, chronic obstructive pulmonary disease, cancer and periodontitis.

11. A method for treating kinase-related disease comprising administering the compound represented by formula 1, the stereoisomer thereof, the hydrate thereof, or the pharmaceutically acceptable salt thereof of claim 1 as an active ingredient to a subject in need thereof.
